SEQUENCE LISTING (65 sequences)

SEQ ID NO: 1 - protein, 509 aa, Homo sapiens
MQSWSRVYCSLAKRGHFNRISHGLQGLSAVPLRTYADQPIDADVTVIGSGPGGYVAAIKAAQLGFKTVCIEKNETLGGTCLNVGCIPSKALLNNSHYYHMAHGKDFASRGIEMSEVRLNLDKMMEQKSTAVKALTGGIAHLFKQNKVVHVNGYGKITGKNQVTATKADGGTQVIDTKNILIATGSEVTPFPGITIDEDTIVSSTGALSLKKVPEKMVVIGAGVIGVELGSVWQRLGADVTAVEFLGHVGGVGIDMEISKNFQRILQKQGFKFKLNTKVTGATKKSDGKIDVSIEAASGGKAEVITCDVLLVCIGRRPFTKNLGLEELGIELDPRGRIPVNTRFQTKIPNIYAIGDVVAGPMLAHKAEDEGIICVEGMAGGAVHIDYNCVPSVIYTHPEVAWVGKSEEQLKEEGIEYKVGKFPFAANSRAKTNADTDGMVKILGQKSTDRVLGAHILGPGAGEMVNEAALALEYGASCEDIARVCHAHPTLSEAFREANLAASFGKSINF

SEQ ID NO: 2 - protein, 253 aa, Homo sapiens
MHSWERLAVLVLLGAAACAAPPRGRILGGREAEAHARPYMASVQLNGAHLCGGVLVAEQWVLSAAHCLEDAADGKVQVLLGAHSLSQPEPSKRLYDVLRAVPHPDSQPDTIDHDLLLLQLSEKATLGPAVRPLPWQRVDRDVAPGTLCDVAGWGIVNHAGRRPDSLQHVLLPVLDRATCNRRTHHDGAITERLMCAESNRRDSCKGDSGGPLVCGGVLEGVVTSGSRVCGNRKKPGIYTRVASYAAWIDSVLA

SEQ ID NO: 3 - protein, 453 aa, Homo sapiens
MALVRALVCCLLTAWHCRSGLGLPVAPAGGRNPPPAIGQFWHVTDLHLDPTYHITDDHTKVCASSKGANASNPGPFGDVLCDSPYQLILSAFDFIKNSGQEASFMIWTGDSPPHVPVPELSTDTVINVITNMTTTIQSLFPNLQVFPALGNHDYWPQDQLPVVTSKVYNAVANLWKPWLDEEAISTLRKGGFYSQKVTTNPNLRIISLNTNLYYGPNIMTLNKTDPANQFEWLESTLNNSQQNKEKVYIIAHVPVGYLPSSQNITAMREYYNEKLIDIFQKYSDVIAGQFYGHTHRDSIMVLSDKKGSPVNSLFVAPAVTPVKSVLEKQTNNPGIRLFQYDPRDYKLLDMLQYYLNLTEANLKGESIWKLEYILTQTYDIEDLQPESLYGLAKQFTILDSKQFIKYYNYFFVSYDSSVTCDKTCKAFQICAIMNLDNISYADCLKQLYIKHNY

SEQ ID NO: 4 - protein, 366 aa, Homo sapiens
MASMAAVLTWALALLSAFSATQARKGFWDYFSQTSGDKGRVEQIHQQKMAREPATLKDSLEQDLNNMNKFLEKLRPLSGSEAPRLPQDPVGMRRQLQEELEEVKARLQPYMAEAHELVGWNLEGLRQQLKPYTMDLMEQVALRVQELQEQLRVVGEDTKAQLLGGVDEAWALLQGLQSRVVHHTGRFKELFHPYAESLVSGIGRHVQELHRSVAPHAPASPARLSRCVQVLSRKLTLKAKALHARIQQNLDQLREELSRAFAGTGTEEGAGPDPQMLSEEVRQRLQAFRQDTYLQIAAFTRAIDQETEEVQQQLAPPPPGHSAFAPEFQQTDSGKVLSKLQARLDDLWEDITHSLHDQGHSHLGDP

SEQ ID NO: 5 - protein, 556 aa, Homo sapiens
MELCGLGLPRPPMLLALLLATLLAAMLALLTQVALVVQVAEAARAPSVSAKPGPALWPLPLLVKMTPNLLHLAPENFYISHSPNSTAGPSCTLLEEAFRRYHGYIFGFYKWHHEPAEFQAKTQVQQLLVSITLQSECDAFPNISSDESYTLLVKEPVAVLKANRVWGALRGLETFSQLVYQDSYGTFTINESTIIDSPRFSHRGILIDTSRHYLPVKIILKTLDAMAFNKFNVLHWHIVDDQSFPYQSITFPELSNKGSYSLSHVYTPNDVRMVIEYARLRGIRVLPEFDTPGHTLSWGKGQKDLLTPCYSRQNKLDSFGPINPTLNTTYSFLTTFFKEISEVFPDQFIHLGGDEVEFKCWESNPKIQDFMRQKGFGTDFKKLESFYIQKVLDIIATINKGSIVWQEVFDDKAKLAPGTIVEVWKDSAYPEELSRVTASGFPVILSAPWYLDLISYGQDWRKYYKVEPLDFGGTQKQKQLFIGGEACLWGEYVDATNLTPRLWPRASAVGERLWSSKDVRDMDDAYDRLTRHRCRMVERGIAAQPLYAGYCNHENM

SEQ ID NO: 6 - protein, 651 aa, Homo sapiens
MARGSAVAWAALGPLLWGCALGLQGGMLYPQESPSRECKELDGLWSFRADFSDNRRRGFEEQWYRRPLWESGPTVDMPVPSSFNDISQDWRLRHFVGWVWYEREVILPERWTQDLRTRVVLRIGSAHSYAIVWVNGVDTLEHEGGYLPFEADISNLVQVGPLPSRLRITIAINNTLTPTTLPPGTIQYLTDTSKYPKGYFVQNTYFDFFNYAGLQRSVLLYTTPTTYIDDITVTTSVEQDSGLVNYQISVKGSNLFKLEVRLLDAENKVVANGTGTQGQLKVPGVSLWWPYLMHERPAYLYSLEVQLTAQTSLGPVSDFYTLPVGIRTVAVTKSQFLINGKPFYFHGVNKHEDADIRGKGFDWPLLVKDFNLLRWLGANAFRTSHYPYAEEVMQMCDRYGIVVIDECPGVGLALPQFFNNVSLHHHMQVMEEVVRRDKNHPAVVMWSVANEPASHLESAGYYLKMVIAHTKSLDPSRPVTFVSNSNYAADKGAPYVDVICLNSYYSWYHDYGHLELIQLQLATQFENWYKKYQKPIIQSEYGAETIAGFHQDPPLMFTEEYQKSLLEQYHLGLDQKRRKYVVGELIWNFADFMTEQSPTRVLGNKKGIFTRQRQPKSAAFLLRERYWKIANETRYPHSVAKSQCLENSLFT

SEQ ID NO: 7 - protein, 375 aa, Homo sapiens
MSTAGKVIKCKAAVLWELKKPFSIEEVEVAPPKAHEVRIKMVAVGICGTDDHVVSGTMVTPLPVILGHEAAGIVESVGEGVTTVKPGDKVIPLAIPQCGKCRICKNPESNYCLKNDVSNPQGTLQDGTSRFTCRRKPIHHFLGISTFSQYTVVDENAVAKIDAASPLEKVCLIGCGFSTGYGSAVNVAKVTPGSTCAVFGLGGVGLSAIMGCKAAGAARIIAVDINKDKFAKAKELGATECINPQDYKKPIQEVLKEMTDGGVDFSFEVIGRLDTMMASLLCCHEACGTSVIVGVPPDSQNLSMNPMLLLTGRTWKGAILGGFKSKECVPKLVADFMAKKFSLDALITHVLPFEKINEGFDLLHSGKSIRTILMF

SEQ ID NO: 8 - protein, 494 aa, Homo sapiens
MFEIKKICCIGAGYVGGPTCSVIAHMCPEIRVTVVDVNESRINAWNSPTLPIYEPGLKEVVESCRGKNLFFSTNIDDAIKEADLVFISVNTPTKTYGMGKGRAADLKYIEACARRIVQNSNGYKIVTEKSTVPVRAAESIRRIFDANTKPNLNLQVLSNPEFLAEGTAIKDLKNPDRVLIGGDETPEGQRAVQALCAVYEHWVPREKILTTNTWSSELSKLAANAFLAQRISSINSISALCEATGADVEEVATAIGMDQRIGNKFLKASVGFGGSCFQKDVLNLVYLCEALNLPEVARYWQQVIDMNDYQRRRFASRIIDSLFNTVTDKKIAILGFAFKKDTGDTRESSSIYISKYLMDEGAHLHIYDPKVPREQIVVDLSHPGVSEDDQVSRLVTISKDPYEACDGAHAVVICTEWDMFKELDYERIHKKMLKPAFIFDGRRVLDGLHNELQTIGFQIETIGKKVSSKRIPYAPSGEIPKFSLQDPPNKKPKV

SEQ ID NO: 9 - protein, 196 aa, Homo sapiens
MDDTLFQLKFTAKQLEKLAKKAEKDSKAEQAKVKKALLQKNVECARVYAENAIRKKNEGVNWLRMASRVDAVASKVQTAVTMKGVTKNMAQVTKALDKALSTMDLQKVSSVMDRFEQQVQNLDVHTSVMEDSMSSATTLTTPQEQVDSLIMQIAEENGLEVLDQLSQLPEGASAVGESSVRSQEDQLSRRLAALRN

SEQ ID NO: 10 - protein, 323 aa, Homo sapiens
MDPKYQRVELNDGHFMPVLGFGTYAPPEVPRNRAVEVTKLAIEAGFRHIDSAYLYNNEEQVGLAIRSKIADGSVKREDIFYTSKLWCTFFQPQMVQPALESSLKKLQLDYVDLYLLHFPMALKPGETPLPKDENGKVIFDTVDLSATWEVMEKCKDAGLAKSIGVSNFNCRQLEMILNKPGLKYKPVCNQVECHPYLNQSKLLDFCKSKDIVLVAHSALGTQRHKLWVDPNSPVLLEDPVLCALAKKHKQTPALIALRYQLQRGVVVLAKSYNEQRIRENIQVFEFQLTSEDMKVLDGLNRNYRYVVMDFLMDHPDYPFSDEY

SEQ ID NO: 11 - protein, 135 aa, Homo sapiens
MPPNLTGYYRFVSQKNMEDYLQALNISLAVRKIALLLKPDKEIEHQGNHMTVRTLSTFRNYTVQFDVGVEFEEDLRSVDGRKCQTIVTWEEEHLVCVQKGEVPNRGWRHWLEGEMLYLELTARDAVCEQVFRKVR

SEQ ID NO: 12 - protein, 411 aa, Homo sapiens
MSPARLRPRLHFCLVLLLLLVVPAAWGCGPGRVVGSRRRPPRKLVPLAYKQFSPNVPEKTLGASGRYEGKIARSSERFKELTPNYNPDIIFKDEENTGADRLMTQRCKDRLNSLAISVMNQWPGVKLRVTEGWDEDGHHSEESLHYEGRAVDITTSDRDRNKYGLLARLAVEAGFDWVYYESKAHVHCSVKSEHSAAAKTGGCFPAGAQVRLESGARVALSAVRPGDRVLAMGEDGSPTFSDVLIFLDREPHRLRAFQVIETQDPPRRLALTPAHLLFTADNHTEPAARFRATFASHVQPGQYVLVAGVPGLQPARVAAVSTHVALGAYAPLTKHGTLVVEDVVASCFAAVADHHLAQLAFWPLRLFHSLAWGSWTPGEGVHWYPQLLYRLGRLLLEEGSFHPLGMSGAGS

SEQ ID NO: 13 - protein, 291 aa, Homo sapiens
MEKLAASTEPQGPRPVLGRESVQVPDDQDFRSFRSECEAEVGWNLTYSRAGVSVWVQAVEMDRTLHKIKCRMECCDVPAETLYDVLHDIEYRKKWDSNVIETFDIARLTVNADVGYYSWRCPKPLKNRDVITLRSWLPMGADYIIMNYSVKHPKYPPRKDLVRAVSIQTGYLIQSTGPKSCVITYLAQVDPKGSLPKWVVNKSSQFLAPKAMKKMYKACLKYPEWKQKHLPHFKPWLHPEQSPLPSLALSELSVQHADSLENIDESAVAESREERMGGAGGEGSDDDTSLT

SEQ ID NO: 14 - protein, 558 aa, Homo sapiens
MYRYLGEALLLSRAGPAALGSASADSAALLGWARGQPAAAPQPGLALAARRHYSEAVADREDDPNFFKMVEGFFDRGASIVEDKLVEDLRTRESEEQKRNRVRGILRIIKPCNHVLSLSFPIRRDDGSWEVIEGYRAQHSQHRTPCKGGIRYSTDVSVDEVKALASLMTYKCAVVDVPFGGAKAGVKINPKNYTDNELEKITRRFTMELAKKGFIGPGIDVPAPDMSTGEREMSWIADTYASTIGHYDINAHACVTGKPISQGGIHGRISATGRGVFHGIENFINEASYMSILGMTPGFGDKTFVVQGFGNVGLHSMRYLHRFGAKCIAVGESDGSIWNPDGIDPKELEDFKLQHGSILGFPKAKPYEGSILEADCDILIPAASEKQLTKSNAPRVKAKIIAEGANGPTTPEADKIFLERNIMVIPDLYLNAGGVTVSYFEWLKNLNHVSYGRLTFKYERDSNYHLLMSVQESLERKFGKHGGTIPIVPTAEFQDRISGASEKDIVHSGLAYTMERSARQIMRTAMKYNLGLDLRTAAYVNAIEKVFKVYNEAGVTFT

SEQ ID NO: 15 - protein, 923 aa, Homo sapiens
MERGLPLLCAVLALVLAPAGAFRNDKCGDTIKIESPGYLTSPGYPHSYHPSEKCEWLIQAPDPYQRIMINFNPHFDLEDRDCKYDYVEVFDGENENGHFRGKFCGKIAPPPVVSSGPFLFIKFVSDYETHGAGFSIRYEIFKRGPECSQNYTTPSGVIKSPGFPEKYPNSLECTYIVFVPKMSEIILEFESFDLEPDSNPPGGMFCRYDRLEIWDGFPDVGPHIGRYCGQKTPGRIRSSSGILSMVFYTDSAIAKEGFSANYSVLQSSVSEDFKCMEALGMESGEIHSDQITASSQYSTNWSAERSRLNYPENGWTPGEDSYREWIQVDLGLLRFVTAVGTQGAISKETKKKYYVKTYKIDVSSNGEDWITIKEGNKPVLFQGNTNPTDVVVAVFPKPLITRFVRIKPATWETGISMRFEVYGCKITDYPCSGMLGMVSGLISDSQITSSNQGDRNWMPENIRLVTSRSGWALPPAPHSYINEWLQIDLGEEKIVRGIIIQGGKHRENKVFMRKFKIGYSNNGSDWKMIMDDSKRKAKSFEGNNNYDTPELRTFPALSTRFIRIYPERATHGGLGLRMELLGCEVEAPTAGPTTPNGNLVDECDDDQANCHSGTGDDFQLTGGTTVLATEKPTVIDSTIQSEFPTYGFNCEFGWGSHKTFCHWEHDNHVQLKWSVLTSKTGPIQDHTGDGNFIYSQADENQKGKVARLVSPVVYSQNSAHCMTFWYHMSGSHVGTLRVKLRYQKPEEYDQLVWMAIGHQGDHWKEGRVLLHKSLKLYQVIFEGEIGKGNLGGIAVDDISINNHISQEDCAKPADLDKKNPEIKIDETGSTPGYEGEGEGDKNISRKPGNVLKTLDPILITIIAMSALGVLLGAVCGVVLYCACWHNGMSERNLSALENYNFELVDGVKLKKDKLNTQSTYSEA

SEQ ID NO: 16 - protein, 177 aa, Homo sapiens
MEICRGLRSHLITLLLFLFHSETICRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 17 - protein, 115 aa, Homo sapiens
MVSRKAVAALLVVHVAAMLASQTEAFVPIFTYGELQRMQEKERNKGQKKSLSVWQRSGEEGPVDPAEPIREEENEMIKLTAPLEIGMRMNSRQLEKYPATLEGLLSEMLPQHAAK

SEQ ID NO: 18 - protein, 1948 aa, Homo sapiens
MAPTWGPGMVSVVGPMGLLVVLLVGGCAAEEPPRFIKEPKDQIGVSGGVASFVCQATGDPKPRVTWNKKGKKVNSQRFETIEFDESAGAVLRIQPLRTPRDENVYECVAQNSVGEITVHAKLTVLREDQLPSGFPNIDMGPQLKVVERTRTATMLCAASGNPDPEITWFKDFLPVDPSASNGRIKQLRSETFESTPIRGALQIESSEETDQGKYECVATNSAGVRYSSPANLYVRELREVRRVAPRFSILPMSHEIMPGGNVNITCVAVGSPMPYVKWMQGAEDLTPEDDMPVGRNVLELTDVKDSANYTCVAMSSLGVIEAVAQITVKSLPKAPGTPMVTENTATSITITWDSGNPDPVSYYVIEYKSKSQDGPYQIKEDITTTRYSIGGLSPNSEYEIWVSAVNSIGQGPPSESVVTRTGEQAPASAPRNVQARMLSATTMIVQWEEPVEPNGLIRGYRVYYTMEPEHPVGNWQKHNVDDSLLTTVGSLLEDETYTVRVLAFTSVGDGPLSDPIQVKTQQGVPGQPMNLRAEARSETSITLSWSPPRQESIIKYELLFREGDHGREVGRTFDPTTSYVVEDLKPNTEYAFRLAARSPQGLGAFTPVVRQRTLQSKPSAPPQDVKCVSVRSTAILVSWRPPPPETHNGALVGYSVRYRPLGSEDPEPKEVNGIPPTTTQILLEALEKWTQYRITTVAHTEVGPGPESSPVVVRTDEDVPSAPPRKVEAEALNATAIRVLWRSPAPGRQHGQIRGYQVHYVRMEGAEARGPPRIKDVMLADAQWETDDTAEYEMVITNLQPETAYSITVAAYTMKGDGARSKPKVVVTKGAVLGRPTLSVQQTPEGSLLARWEPPAGTAEDQVLGYRLQFGREDSTPLATLEFPPSEDRYTASGVHKGATYVFRLAARSRGGLGEEAAEVLSIPEDTPRGHPQILEAAGNASAGTVLLRWLPPVPAERNGAIVKYTVAVREAGALGPARETELPAAAEPGAENALTLQGLKPDTAYDLQVRAHTRRGPGPFSPPVRYRTFLRDQVSPKNFKVKMIMKTSVLLSWEFPDNYNSPTPYKIQYNGLTLDVDGRTTKKLITHLKPHTFYNFVLTNRGSSLGGLQQTVTAWTAFNLLNGKPSVAPKPDADGFIMVYLPDGQSPVPVQSYFIVMVPLRKSRGGQFLTPLGSPEDMDLEELIQDISRLQRRSLRHSRQLEVPRPYIAARFSVLPPTFHPGDQKQYGGFDNRGLEPGHRYVLFVLAVLQKSEPTFAASPFSDPFQLDNPDPQPIVDGEEGLIWVIGPVLAVVFIICIVIAILLYKNKPDSKRKDSEPRTKCLLNNADLAPHHPKDPVEMRRINFQTPDSGLRSPLREPGFHFESMLSHPPIPIADMAEHTERLKANDSLKLSQEYESIDPGQQFTWEHSNLEVNKPKNRYANVIAYDHSRVILQPIEGIMGSDYINANYVDGYRCQNAYIATQGPLPETFGDFWRMVWEQRSATIVMMTRLEEKSRIKCDQYWPNRGTETYGFIQVTLLDTIELATFCVRTFSLHKNGSSEKREVRQFQFTAWPDHGVPEYPTPFLAFLRRVKTCNPPDAGPIVVHCSAGVGRTGCFIVIDAMLERIKPEKTVDVYGHVTLMRSQRNYMVQTEDQYSFIHEALLEAVGCGNTEVPARSLYAYIQKLAQVEPGEHVTGMELEFKRLANSKAHTSRFISANLPCNKFKNRLVNIMPYESTRVCLQPIRGVEGSDYINASFIDGYRQQKAYIATQGPLAETTEDFWRMLWENNSTIVVMLTKLREMGREKCHQYWPAERSARYQYFVVDPMAEYNMPQYILREFKVTDARDGQSRTVRQFQFTDWPEQGVPKSGEGFIDFIGQVHKTKEQFGQDGPISVHCSAGVGRTGVFITLSIVLERMRYEGVVDIFQTVKMLRTQRPAMVQTEDEYQFCYQAALEYLGSFDHYAT

SEQ ID NO: 19 - protein, 402 aa, Homo sapiens
MESRGPLATSRLLLLLLLLLLRHTRQGWALRPVLPTQSAHDPPAVHLSNGPGQEPIAVMTFDLTKITKTSSSFEVRTWDPEGVIFYGDTNPKDDWFMLGLRDGRPEIQLHNHWAQLTVGAGPRLDDGRWHQVEVKMEGDSVLLEVDGEEVLRLRQVSGPLTSKRHPIMRIALGGLLFPASNLRLPLVPALDGCLRRDSWLDKQAEISASAPTSLRSCDVESNPGIFLPPGTQAEFNLRDIPQPHAEPWAFSLDLGLKQAAGSGHLLALGTPENPSWLSLHLQDQKVVLSSGSGPGLDLPLVLGLPLQLKLSMSRVVLSQGSKMKALALPPLGLAPLLNLWAKPQGRLFLGALPGEDSSTSFCLNGLWAQGQRLDVDQALNRSHEIWTHSCPQSPGNGTDASH

SEQ ID NO: 20 - protein, 692 aa, Homo sapiens
MGTVSSRRSWWPLPLLLLLLLLLGPAGARAQEDEDGDYEELVLALRSEEDGLAEAPEHGTTATFHRCAKDPWRLPGTYVVVLKEETHLSQSERTARRLQAQAARRGYLTKILHVFHGLLPGFLVKMSGDLLELALKLPHVDYIEEDSSVFAQSIPWNLERITPPRYRADEYQPPDGGSLVEVYLLDTSIQSDHREIEGRVMVTDFENVPEEDGTRFHRQASKCDSHGTHLAGVVSGRDAGVAKGASMRSLRVLNCQGKGTVSGTLIGLEFIRKSQLVQPVGPLVVLLPLAGGYSRVLNAACQRLARAGVVLVTAAGNFRDDACLYSPASAPEVITVGATNAQDQPVTLGTLGTNFGRCVDLFAPGEDIIGASSDCSTCFVSQSGTSQAAAHVAGIAAMMLSAEPELTLAELRQRLIHFSAKDVINEAWFPEDQRVLTPNLVAALPPSTHGAGWQLFCRTVWSAHSGPTRMATAVARCAPDEELLSCSSFSRSGKRRGERMEAQGGKLVCRAHNAFGGEGVYAIARCCLLPQANCSVHTAPPAEASMGTRVHCHQQGHVLTGCSSHWEVEDLGTHKPPVLRPRGQPNQCVGHREASIHASCCHAPGLECKVKEHGIPAPQEQVTVACEEGWTLTGCSALPGTSHVLGAYAVDNTCVVRSRDVSTTGSTSEGAVTAVAICCRSRHLAQASQELQ

SEQ ID NO: 21 - protein, 445 aa, Homo sapiens
MAELKYISGFGNECSSEDPRCPGSLPEGQNNPQVCPYNLYAEQLSGSAFTCPRSTNKRSWLYRILPSVSHKPFESIDEGQVTHNWDEVDPDPNQLRWKPFEIPKASQKKVDFVSGLHTLCGAGDIKSNNGLAIHIFLCNTSMENRCFYNSDGDFLIVPQKGNLLIYTEFGKMLVQPNEICVIQRGMRFSIDVFEETRGYILEVYGVHFELPDLGPIGANGLANPRDFLIPIAWYEDRQVPGGYTVINKYQGKLFAAKQDVSPFNVVAWHGNYTPYKYNLKNFMVINSVAFDHADPSIFTVLTAKSVRPGVAIADFVIFPPRWGVADKTFRPPYYHRNCMSEFMGLIRGHYEAKQGGFLPGGGSLHSTMTPHGPDADCFEKASKVKLAPERIADGTMAFMFESSLSLAVTKWGLKASRCLDENYHKCWEPLKSHFTPNSRNPAEPN

SEQ ID NO: 22 - DNA, 23 bp, synthetic construct
  source 1..23: mol_type = other DNA
aagtcacacaagatgctacaccg

SEQ ID NO: 23 - DNA, 21 bp, synthetic construct
  source 1..21: mol_type = other DNA
tcagcgaagacatctggtgcc

SEQ ID NO: 24 - DNA, 22 bp, synthetic construct
  source 1..22: mol_type = other DNA
ctcatcagccactggaaaggca

SEQ ID NO: 25 - DNA, 22 bp, synthetic construct
  source 1..22: mol_type = other DNA
gactcgtgaagtcagcctgaaa

SEQ ID NO: 26 - DNA, 22 bp, synthetic construct
  source 1..22: mol_type = other DNA
accttcgacgtgtccatcctga

SEQ ID NO: 27 - DNA, 22 bp, synthetic construct
  source 1..22: mol_type = other DNA
tcctccacgaagtggttcacca

SEQ ID NO: 28 - DNA, 22 bp, synthetic construct
  source 1..22: mol_type = other DNA
tgggaaggatgccaccaatgtg

SEQ ID NO: 29 - DNA, 22 bp, synthetic construct
  source 1..22: mol_type = other DNA
gcgatagaactcagatgctgcc

SEQ ID NO: 30 - DNA, 22 bp, synthetic construct
  source 1..22: mol_type = other DNA
accttcgacgtgtccatcctga

SEQ ID NO: 31 - DNA, 22 bp, synthetic construct
  source 1..22: mol_type = other DNA
tcctccacgaagtggttcacca

SEQ ID NO: 32 - DNA, 22 bp, synthetic construct
  source 1..22: mol_type = other DNA
ggagttgctcttggtgttgtgg

SEQ ID NO: 33 - DNA, 22 bp, synthetic construct
  source 1..22: mol_type = other DNA
ggataaagccgaccctcttcca

SEQ ID NO: 34 - DNA, 22 bp, synthetic construct
  source 1..22: mol_type = other DNA
ggatggtgtcaactatgccagc

SEQ ID NO: 35 - DNA, 22 bp, synthetic construct
  source 1..22: mol_type = other DNA
cacgctcccttcctcttgatgt

SEQ ID NO: 36 - DNA, 23 bp, synthetic construct
  source 1..23: mol_type = other DNA
ggtaactttctcacaggccttgg

SEQ ID NO: 37 - DNA, 22 bp, synthetic construct
  source 1..22: mol_type = other DNA
tccctctgtaacaggtgccttg

SEQ ID NO: 38 - DNA, 22 bp, synthetic construct
  source 1..22: mol_type = other DNA
cacttcagtggcagatgtggtg

SEQ ID NO: 39 - DNA, 23 bp, synthetic construct
  source 1..23: mol_type = other DNA
gcagtggaaatctgctctgacag

SEQ ID NO: 40 - DNA, 22 bp, synthetic construct
  source 1..22: mol_type = other DNA
gccaagaagtcagcctactgcc

SEQ ID NO: 41 - DNA, 22 bp, synthetic construct
  source 1..22: mol_type = other DNA
cttctggatagcggtccgttca

SEQ ID NO: 42 - DNA, 22 bp, synthetic construct
  source 1..22: mol_type = other DNA
ctgaggatctcaagcgcagcta

SEQ ID NO: 43 - DNA, 22 bp, synthetic construct
  source 1..22: mol_type = other DNA
agcaggactgtggctccaaaac

SEQ ID NO: 44 - DNA, 22 bp, synthetic construct
  source 1..22: mol_type = other DNA
caggacaagagcctatcgctgt

SEQ ID NO: 45 - DNA, 23 bp, synthetic construct
  source 1..23: mol_type = other DNA
gtcatccttagggttggtatccc

SEQ ID NO: 46 - DNA, 22 bp, synthetic construct
  source 1..22: mol_type = other DNA
agtctgtcgtccagtagcacca

SEQ ID NO: 47 - DNA, 22 bp, synthetic construct
  source 1..22: mol_type = other DNA
actggagccatactcatccgag

SEQ ID NO: 48 - DNA, 20 bp, synthetic construct
  source 1..20: mol_type = other DNA
cctgccccctgccaacacct

SEQ ID NO: 49 - DNA, 23 bp, synthetic construct
  source 1..23: mol_type = other DNA
tgtgacatcctggtgataaagcc

SEQ ID NO: 50 - DNA, 22 bp, synthetic construct
  source 1..22: mol_type = other DNA
tcggcgaggatagttctggaag

SEQ ID NO: 51 - DNA, 22 bp, synthetic construct
  source 1..22: mol_type = other DNA
gaccacaggataagtcaccgag

SEQ ID NO: 52 - DNA, 22 bp, synthetic construct
  source 1..22: mol_type = other DNA
agcctgcgaaagccttttggtg

SEQ ID NO: 53 - DNA, 23 bp, synthetic construct
  source 1..23: mol_type = other DNA
ggcttcacattcagcaaacctgg

SEQ ID NO: 54 - DNA, 23 bp, synthetic construct
  source 1..23: mol_type = other DNA
acgagaggatgataaactggtgg

SEQ ID NO: 55 - DNA, 21 bp, synthetic construct
  source 1..21: mol_type = other DNA
gcgaacttcagtccaggtcaa

SEQ ID NO: 56 - DNA, 22 bp, synthetic construct
  source 1..22: mol_type = other DNA
acttctggaggcatcgcaagca

SEQ ID NO: 57 - DNA, 22 bp, synthetic construct
  source 1..22: mol_type = other DNA
aggttccagaggaggctacaag

SEQ ID NO: 58 - DNA, 22 bp, synthetic construct
  source 1..22: mol_type = other DNA
caccattggcaatgagcggttc

SEQ ID NO: 59 - DNA, 22 bp, synthetic construct
  source 1..22: mol_type = other DNA
aggtctttgcggatgtccacgt

SEQ ID NO: 60 - DNA, 21 bp, synthetic construct
  source 1..21: mol_type = other DNA
cgagggcacttgtgagaagcg

SEQ ID NO: 61 - DNA, 22 bp, synthetic construct
  source 1..22: mol_type = other DNA
tgttcatggtgctgtccacgtg

SEQ ID NO: 62 - DNA, 22 bp, synthetic construct
  source 1..22: mol_type = other DNA
cctacactctcctccatcttgc

SEQ ID NO: 63 - DNA, 22 bp, synthetic construct
  source 1..22: mol_type = other DNA
cctggcatagatgtagccctca

SEQ ID NO: 64 - DNA, 22 bp, synthetic construct
  source 1..22: mol_type = other DNA
ccgaagcaagattgcagatggc

SEQ ID NO: 65 - DNA, 22 bp, synthetic construct
  source 1..22: mol_type = other DNA
gtgagctttccaaggctggttg

(19) United States
(12) Patent Application Publication
(10) Pub. No.: US 2026/0202417 A1
(43) Pub. Date: Jul. 16, 2026

(54) PROTEOMICS MARKERS OF STEATOSIS

(71) Applicants: Vanderbilt University, Nashville, TN (US); Northwestern University, Evanston, IL (US)

(72) Inventors: Ravi Shah, Nashville, TN (US); Ravi Kalhan, Evanston, IL (US); Andrew Perry, Nashville, TN (US); Jennifer Below, Nashville, TN (US); Eric Gamazon, Nashville, TN (US)

(21) Appl. No.: 19/443,584

(22) Filed: Jan. 8, 2026

Related U.S. Application Data
(60) Provisional application No. 63/743,027, filed on Jan. 8, 2025.

(51) Int. Cl. G01N 33/68; G16H 50/20; G16H 50/30

(52) U.S. Cl. CPC G01N 33/6848; G16H 50/20; G16H 50/30; G01N 2570/00; G01N 2800/085; G01N 2800/50; G01N 2800/52

(57) ABSTRACT

Methods, kits, and computer-implemented systems are provided for diagnosing or predicting metabolic dysfunction-associated steatotic liver disease (MASLD) or progression thereof in a subject.

### RELATED APPLICATIONS

This application claims priority from U.S. Provisional Application Ser. No. 63/743,027 filed Jan. 8, 2025, the entire disclosure of which is incorporated herein by this reference.

### GOVERNMENT INTEREST

This invention was made with government support under R01HL122477 awarded by National Institutes of Health. The government has certain rights in the invention.

### REFERENCE TO AN ELECTRONIC SEQUENCE LISTING

The contents of the electronic sequence listing (VU24044 Sequence Listing.xml; Size: 67979 bytes; and Date of Creation: Jan. 8, 2026) is herein incorporated by reference in its entirety.

### TECHNICAL FIELD

The presently-disclosed subject matter generally relates the fields of medicine, proteomics, and fatty liver disease. More particularly, the disclosure relates to methods of diagnosing and treating diseases steatosis.

### INTRODUCTION

Metabolic dysfunction-associated steatotic liver disease (MASLD) represents a rapidly escalating global health challenge, affecting a significant proportion of the population and contributing to liver failure, cardiovascular disease, and other chronic conditions. Despite its prevalence, current diagnostic strategies remain inadequate for early detection and risk stratification. Imaging modalities such as magnetic resonance imaging (MRI) and computed tomography (CT) are considered gold standards but are expensive, resource-intensive, and impractical for widespread screening. Ultrasound, while more accessible, suffers from poor sensitivity and requires a substantial hepatic fat burden, typically well over 5% and closer to 20-30% of hepatocytes infiltrated with fat, before changes become detectable, thereby missing early disease stages when intervention could be most effective.

Circulating biomarkers and liver enzyme measurements (e.g., AST, ALT) are nonspecific and often reflect advanced disease rather than early pathological changes. Genetic studies and invasive liver biopsies, though informative, fail to capture dynamic environmental and behavioral influences that drive MASLD progression. Furthermore, emerging proteomic approaches have been constrained by limited sample sizes, lack of tissue-specific prioritization, and insufficient integration of systemic and hepatic processes. These limitations collectively hinder the development of scalable, non-invasive tools for early detection, prognostication, and therapeutic targeting.

The inability of current low- or non-invasive methods for to detect disease at its earliest stages, to link circulating molecular states with dynamic hepatic tissue changes, and to provide actionable insights for diverse populations underscores a critical gap in the field. Over the past two decades, the prevalence of MASLD has surged globally, approximately doubling in incidence, paralleling increases in obesity and metabolic dysfunction. This accelerating trend highlights the urgency for improved strategies that enable timely identification of at-risk individuals, enhance population-level screening, and inform precision therapies aimed at halting disease progression before irreversible damage occurs.

Accordingly, there remains a need in the art for improved, low- and non-invasive methods to for prediction, assessment, detection, risk stratification, and mechanistic understanding of MASLD.

### SUMMARY

The presently-disclosed subject matter meets some or all of the above-identified needs, as will become evident to those of ordinary skill in the art after a study of information provided in this document.

This Summary describes several embodiments of the presently-disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

The presently-disclosed subject matter meets some or all of the above-identified needs, as will become evident to those of ordinary skill in the art after a study of information provided in this document.

In certain embodiments, the presently-disclosed subject matter provides methods for diagnosing or predicting metabolic dysfunction-associated steatotic liver disease (MASLD) or progression thereof in a subject, as well as methods for assessing hepatic steatosis. These methods generally comprise obtaining a biological sample, such as plasma or serum, from the subject; quantifying concentrations of a plurality of MASLD-associated biomarkers selected from defined panels; and generating a MASLD risk score or classification metric based on the quantified concentrations. The MASLD risk score may optionally integrate clinical covariates such as age, body mass index, and fasting glucose level, and can be compared to a predetermined threshold determined using receiver operating characteristic (ROC) analysis to identify the subject as having MASLD or an increased risk thereof.

In some embodiments, biomarker quantification is performed using proteomic techniques such as multiplexed mass spectrometry or immunoassay-based platforms, enabling simultaneous detection of multiple biomarkers in a single analytical run. Multiplexing strategies may include isobaric labeling reagents such as tandem mass tags (TMT) or equivalent approaches. Automated analytical devices configured with calibration curves tailored to MASLD biomarker ranges may be employed to improve accuracy and reproducibility. In certain embodiments, the MASLD risk score is validated using a liver-on-a-chip system configured to model hepatic steatosis under controlled conditions.

Additional embodiments include kits for diagnosing or predicting MASLD, comprising reagents for detection of MASLD-associated biomarkers and instructions, provided in printed form or stored on a non-transitory computer-readable medium, for calculating a MASLD risk score using regression-based or machine learning algorithms trained on MASLD phenotypes. Kits may include multiplexed assay cartridges, calibration standards, and optional integration with automated immunoassay devices.

Further embodiments provide computer-implemented methods for diagnosing or predicting MASLD risk. These methods comprise receiving as input quantified biomarker concentrations, applying a multivariable model trained on MASLD phenotypes, such as regression-based algorithms or machine learning approaches (e.g., gradient boosting, random forest, neural networks), and outputting a MASLD risk score via a graphical user interface or electronic report. Computing devices may include local workstations or cloud-based servers configured to execute instructions stored on a non-transitory computer-readable medium.

In certain embodiments, the disclosed methods further comprise administering a therapeutic agent or intervention for treating MASLD when the MASLD risk score exceeds the threshold value. Representative therapeutic agents include SGLT2 inhibitors, GLP-1 receptor agonists, dual GIP/GLP-1 receptor agonists, thiazolidinediones, biguanides, THR-β agonists, FGF analogs, FXR agonists, PPAR agonists, ACC inhibitors, DGAT2 inhibitors, CCR2/CCR5 antagonists, antioxidants, and statins. Clinical management may also include bariatric surgery and structured lifestyle interventions.

The presently-disclosed subject matter also encompasses embodiments integrating proteomic and transcriptomic data to improve predictive performance, as well as methods for establishing a role for gene targets in MASLD pathophysiology through correlation of phenotypic and gene expression data.

### BRIEF DESCRIPTION OF THE DRAWINGS

The features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIG. 1 illustrates the integrative workflow used to identify and validate circulating biomarkers of hepatic steatosis in metabolic dysfunction-associated steatotic liver disease (MASLD).

FIG. 2A-2F: Proteins related to hepatic steatosis are primarily expressed in the liver and identify pathways of metabolism. (FIG. 2A) Volcano plot of proteins associated with hepatic steatosis after adjustment for age, gender, race, and BMI. For visualization, proteins with an FDR<5% in CARDIA derivation subsample are visualized with the beta coefficient and p values presented coming from models using the CARDIA validation subsample. (FIG. 2B) Heatmap of the top 25 positively associated and top 25 negatively associated proteins with hepatic steatosis in the CARDIA validation sample. MASLD is defined as CT liver attenuation <40 HU. (FIG. 2C) and (FIG. 2D) Tissue expression analysis at the transcriptional (FIG. 2C) and tissue protein (FIG. 2D) level for proteins related to hepatic steatosis in CARDIA using the full SomaScan 7k platform as the background, demonstrated enrichment of proteins expressed in the liver. (E) KEGG and Reactome pathway analysis. (FIG. 2F) Hub gene analysis of significant proteins associated with liver attenuation showing the hub genes (≥5 connections; circles) and all proteins with high confidence connections to the hub genes (rectangles).

FIG. 3A-3B: Stability and validation of regression estimates in CARDIA. (FIG. 3A) There was high correlation between beta coefficients from linear models relating individual aptamers to liver attenuation adjusted age, gender, race, BMI adjusted with beta coefficients from the “full” adjusted (age, gender, race, BMI, alcoholic drinks per week, systolic blood pressure, hypertension and cholesterol medication use, diabetes, pack years, total cholesterol, high-density lipoprotein, estimated glomerular filtration rate, and moderate-vigorous physical activity) in CARDIA validate sample. (FIG. 3B) There was moderate correlation of beta coefficients from linear models relating individual aptamers to liver attenuation adjusted age, gender, race, BMI between CARDIA derivation and validation subsamples. Shade of dots represent the density of observations.

FIG. 4A-4D: Development of a proteomic score of hepatic steatosis and its relation with clinical outcomes. (FIG. 4A) A protein score of liver attenuation by CT (less attenuation more steatosis) demonstrated moderate correlation with the parent variable in both CARDIA derivation and validation samples. (FIG. 4B) Replication of the association between a protein score of liver attenuation and MRI-based measure of hepatic steatosis (proton density fat fraction: higher~more steatosis, opposite directionality as with CT based liver attenuation) in UK Biobank. (FIG. 4C) The protein score is related to controlled attenuation parameter (higher value~more steatosis) in CCHC. (FIG. 4D) Forest plot of associations with clinical outcomes in UK Biobank along with C-index comparisons of models with and without the protein score. P values reported are for comparisons of C-indices.

FIG. 5: Correlation of MASLD score with hepatic steatosis in CARDIA participants meeting MASLD clinical criteria. Included are participants in the CARDIA validation subsample with MASLD clinical criteria. Shade of dots represent the density of observations.

FIG. 6A-6H: Relation of the MASLD score with age, gender, race, BMI, and alcohol use. (FIG. 6A, 6B) Protein score of liver attenuation is not strongly related to age. (FIG. 6C, 6D) Protein score of liver attenuation not significantly different across gender and race. (FIG. 6E, 6F) There is a weak correlation between the protein score of liver attenuation and body mass index (BMI). (FIG. 6G, 6H) Protein score of liver attenuation does not vary greatly by alcohol use.

FIG. 7A-7C: Performance of a clinically translatable protein score of liver attenuation. Recognizing the cumbersome nature to measure hundreds of proteins in a clinical setting to generate a protein score of liver attenuation, a truncated version was created including the top 21 proteins. Good model fit was observed in the CARDIA derivation and validation subsamples (FIG. 7A), as well as in UK Biobank (FIG. 7B). Associations with clinical outcomes were comparable between the full protein score and the truncated score in UK Biobank (FIG. 7C).

FIG. 8A-8B: Relation of a protein score of liver attenuation with ultrasound-based measures of fibrosis. Using data from CCHC weak correlations were observed between measures of fibrosis and the protein score of liver attenuation. (FIG. 8A) FIB-4 index. (FIG. 8B) Liver stiffness as measured by acoustic radiation force impulse elastography.

FIG. 9: Correlation of a protein score of liver attenuation with hepatic steatosis in obese CARDIA participants. Using data from the CARDIA validation sample (N=803; N normal/overweight=447, N obese=356), a stronger correlation was observed between hepatic steatosis and the protein score of liver attenuation among obese (body mass index ≥30 kg/m²) participants.

FIG. 10: Upset plot showing the intersection of MASLD-derived proteins and expressed gene lists from the Visium and scRNAseq datasets. The final gene set consisted of 198 protein-gene overlaps that were derived the MASLD proteome and were both expressed in scRNAseq and Visium data.

FIG. 11A-11H: Transcriptional architecture of the hepatic steatosis proteome. (FIG. 11A) Uniform manifold approximation projection (UMAP) of integrated single-nuclear and single-cell RNA sequencing in steatotic and healthy liver, colored by a composite expression score (derived from gene expression of implicated target proteins, N=19, single-nuclear RNA-seq N=5, single-cell RNA-seq N=14). (FIG. 11B, 11C) UMAP of spatial transcriptomic data (Visium, 5 samples from 4 individuals) in the liver colored by liver pathology diagnosis (healthy vs. fatty, FIG. 11B) and the composite expression score (FIG. 11C). (FIG. 11D) Violin plot comparing composite expression score across Visium spots by fatty vs. healthy state (Wilcoxon rank-sum). (FIG. 11E) Representative images of healthy and steatotic hematoxylin-eosin-stained liver tissue overlaid with Visium spots, colored by composite expression score, demonstrating increased activity of implicated targets in steatotic regions. Images are from livercellatlas.org (Guilliams et. al³⁴). All sections presented herein are shown in FIG. 7. (FIG. 11F) Composite expression across liver zonation groups. (FIG. 11G) Differential expression of implicated targets between healthy and fatty liver (Visium) versus circulating proteomic regression coefficient. A more positive proteomic coefficient indicates less liver fat, and a more positive log₂ fold-change indicates greater expression of a given transcript in healthy (non-steatotic) liver. Black dots are targets that were considered as differentially expressed using spatial data (see text). For targets with multiple aptamers (e.g., IGFBP2 has multiple SomaScan aptamers), the mean regression coefficient for that target is presented. (FIG. 11H) Gene expression of significantly different implicated targets between healthy and steatotic regions (Visium), liver zonation (Visium) and across cell types (single-cell RNA sequencing).

FIG. 12A-12D: Cell-specific and spatial architecture of the MASLD proteome. (FIG. 12A) Gene expression of 198 implicated targets by the circulating MASLD proteome (in CARDIA) and were also expressed in both scRNA-seq and Visium datasets across cell types using only the liver cell atlas scRNA-seq dataset. (FIG. 12B) Hematoxylin-eosin stained healthy and steatotic liver tissue, overlaid with composite expression score. Two examples were shown in the main text; this figure includes all sections reported in the initial work. Images are from livercellatlas.org (Guilliams et. al³⁴). (FIG. 12C) Hematoxylin-eosin stained healthy and steatotic liver tissues, overlaid with gene expression of the top three gene targets implicated by the CARDIA model and differentially expressed in healthy and steatotic liver tissue. Images are from livercellatlas.org (Guilliams et. al³⁴). (FIG. 12D) Violin plots showing the top 4 model candidate gene expression across spots from healthy and fatty liver samples (Visium) and a representative model target that was not differentially expressed using the differential expression criteria (IGF1).

FIG. 13: Comparison of log 2 fold change with negative binomial mixed models in spatial transcriptomic analysis. The effect size estimates of the differential expression analysis were compared to coefficients from negative binomial mixed models with sample as random effect. High agreement was observed between log₂ fold change and the negative binomial mixed model coefficients for all 198 model targets (all points) and for the 33 differentially expressed genes (gray points). Pearson r reported.

FIG. 14A-14B: Transcriptional heterogeneity of spatial targets in human liver across steatosis stages. (FIG. 14A) Bulk transcript log₂ fold change in human liver (over control samples without histologic steatosis) for those genes (among 33 significant on spatial studies) that were significantly differentially expressed in at least one comparison (stage 1 vs. control; stage 2 vs. control; stage 3 vs. control). Of the 33 genes passed forward for assessment in bulk transcriptomics, 12 were not significantly expressed in any of the stages of steatosis (by FDR adjusted P<0.05) and were not included in visualization. The “liver attenuation beta” represents the regression coefficient against liver attenuation in the CARDIA derivation sample. A positive coefficient indicates a greater protein level is related to higher attenuation (lower steatosis); a negative coefficient indicates a greater protein level is related to lower attenuation (higher steatosis). For proteins with multiple aptamers (e.g., IGFBP2 has multiple SomaScan aptamers), the mean regression coefficient for that protein is presented. This analysis excluded individuals with stage F4 fibrosis, given differences in hepatic physiology at this stage of decompensation. (FIG. 14B) Violin plots of example gene expression (in log₂ counts per million) for genes that displayed a “concordant” directionality between the proteome and the bulk transcriptome (top and middle panel) and “discordant” directionality between proteome and bulk transcriptome. Differential gene expression analysis was performed using limma-voom with the protein-coding genes using an FDR of 5% (Benjamini-Hochberg). * p<0.05, ** p<0.01, *** p<0.001, **** p<0.0001.

FIG. 15: Transcriptional heterogeneity of spatial targets in human liver across fibrosis stages. Bulk transcript log₂ fold change in human liver (over control samples without histologic steatosis) for the 33 genes significant on spatial studies. The “liver attenuation beta” represents the regression coefficient against liver attenuation in the CARDIA derivation sample. A positive coefficient indicates a greater protein level is related to higher attenuation (lower steatosis); a negative coefficient indicates a greater protein level is related to lower attenuation (higher steatosis). For proteins with multiple aptamers (e.g., IGFBP2 has multiple SomaScan aptamers), the mean regression coefficient for that protein is presented.

FIG. 16A-16F: Transcriptional and proteomic architecture of MASLD on a humanized liver-on-a-chip (LOC). (FIG. 16A) and (FIG. 16B) show the structure and experimental design of MASLD induction on the LOC. (FIG. 16C) Successful MASLD model generation on a representative LOC. On the left, lipid droplet accumulation was visualized after 5-day treatment period of FAs. Representative brightfield and fluorescent confocal images of the LOC cells (scale bar=100 m). DAPI (nuclear) and LipidSpot (lipid droplet) stains are shown. Arrows represent lipid droplet accumulation. (FIG. 16D) mRNA expression of canonical genes implicated in steatosis demonstrate expression patterns consistent with MASLD in both hepatocytes and NPCs. A total of 6 chips were included (3 FA and 3 control). Results were analyzed by an unpaired t test and expressed as mean±standard error of 3 independent experiments. Each data point represents the average of 3 technical replicates. Control (left) and FA treated (right). Relative expression is shown as fold change (delta-delta CT) relative to control, normalized to β-ACTIN expression. (FIG. 16E) mRNA expression of top genes on the LOC that were prioritized by proteomic and transcriptomic studies (see text). Breaks in Y axis are presented given disparate expression of some genes (e.g., HMGCS1 had low expression in non-hepatocytes, while HSPA1A was expressed at low levels in hepatocytes). ME1, CTSZ and DEFB1 were not expressed in hepatocytes; CDA, SHBG, IL1RAP and IGFBP2 were not expressed in NPCs. (FIG. 16F) Secretory protein expression of top targets (Both cell types: HMGCS1, SERPINE1, PYGL; NPC specific: DEFB1, CTSZ; Hepatocyte specific: ACY1 and AKR1C4) on the LOC was consistent with the mRNA expression. A total of 8 chips were included (4 FA and 4 control). Results were analyzed by an unpaired t test and expressed as mean±standard error of 4 independent experiments. Each data point represents the average of 2 technical replicates. Control (Ctrl; left) and FA treated (FA; right). Abbreviations: ne, not expressed (raw Ct>40); nd, not detected; ns, non-significant; *, p<0.05; **, p<0.01; ***, p<0.001; ****, p<0.0001.

FIG. 17A-17B: Gene expression of top 2 implicated targets by the circulating MASLD proteome (in CARDIA). (FIG. 17A) mRNA expression of canonical genes (IRS1 and IRS2) implicated in liver steatosis exhibit expression patterns consistent with MASLD in both hepatocytes and NPCs. (FIG. 17B) mRNA expression of hepatocytes specific (not expressed in NPCs) top targets (ACY1, and AKR1C4) on the LOC that were prioritized by proteomic study (CARDIA derivation, see text); total of 8 chips were included (4 FA and 4 control). Results were analyzed by an unpaired t test and expressed as mean±standard error of 4 independent experiments. Each data point represents the average of 3 technical replicates. Control (left) and FA treated (right). Relative expression is shown as fold change (delta-delta CT) relative to control, normalized to β-ACTIN expression. Abbreviations: ne, not expressed (raw Ct>40); ns, non-significant; ***, p<0.001; ****, p<0.0001. Related to FIG. 5.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The presently disclosed subject matter includes methods for diagnosing or predicting metabolic dysfunction-associated steatotic liver disease (MASLD) or progression thereof in a subject, methods of assessing hepatic steatosis in a subject, kits for diagnosing or predicting MASLD in a subject, and computer-implemented methods of diagnosing or predicting risk of MASLD in a subject. Embodiments of the presently disclosed subject matter make use of quantified concentrations of a plurality of biomarkers obtained from a biological sample, such as plasma or serum, as disclosed herein.

The plurality of biomarkers can be selected from a defined group of MASLD-associated biomarkers, including MASLD-associated proteins, as defined herein. Exemplary biomarker panels are provided in Table 1 and Table 5. These panels include proteins involved in lipid metabolism, inflammation, and cellular signaling, and were selected based on statistical associations with MASLD status in human cohorts. Quantification of these biomarkers can be performed using proteomic techniques such as mass spectrometry or immunoassay-based platforms, enabling simultaneous detection of multiple biomarkers in a single analytical run.

TABLE 1
Protein and Gene Targets Associated with Metabolic Dysfunction-
Associated Steatotic Liver Disease (MASLD)

| UniProt | | | | |
|---|---|---|---|---|
| Accession | Entrez Gene | | | SEQ ID |
| Number | Symbol | Target Full Name | Coefficient | NO: |
| P09622 | DLD | Dihydrolipoyl dehydrogenase, | 0.033571612 | 1 |
| | | mitochondrial (DLDH) | | |
| P00746 | CFD | Complement factor D (Factor D) | 0.05578948 | 2 |
| Q92484 | SMPDL3A | Acid sphingomyelinase-like | −0.054487214 | 3 |
| | | phosphodiesterase 3a (ASM3A) | | |
| Q6Q788 | APOA5 | Apolipoprotein A-V (Apo A-V) | −0.037940579 | 4 |
| P07686 | HEXB | Beta-hexosaminidase subunit beta | −0.069891732 | 5 |
| | | (Hexosaminidase B) | | |
| P08236 | GUSB | Beta-glucuronidase (BGLR) | −0.050850877 | 6 |
| P07327 | ADH1A | Alcohol dehydrogenase 1A | −0.053654844 | 7 |
| | | (ADH1A) | | |
| O60701 | UGDH | UDP-glucose 6-dehydrogenase | −0.033651416 | 8 |
| | | (UGDH) | | |
| Q9HD42 | CHMP1A | Charged multivesicular body | 0.037942883 | 9 |
| | | protein 1a (CHM1A) | | |
| P17516 | AKR1C4 | Aldo-keto reductase family 1 | −0.036702439 | 10 |
| | | member C4 (AK1C4) | | |
| P82980 | RBP5 | Retinol-binding protein 5 (RBP-III) | −0.04201165 | 11 |
| Q14623 | IHH | Indian hedgehog protein (ihh) | 0.037928756 | 12 |
| Q9Y365 | STARD10 | START domain-containing protein | −0.046580231 | 13 |
| | | 10 (STA10) | | |
| P00367 | GLUD1 | Glutamate dehydrogenase 1, | −0.044632263 | 14 |
| | | mitochondrial (DHE3) | | |
| O14786 | NRP1 | Neuropilin-1 (NRP1) | −0.067779015 | 15 |
| P18510 | IL1RN | Interleukin-1 receptor antagonist | −0.037641686 | 16 |
| | | protein (IL-1Ra) | | |
| P12872 | MLN | Promotilin (MOTI) | −0.040651491 | 17 |
| Q13332 | PTPRS | Receptor-type tyrosine-protein | 0.07763276 | 18 |
| | | phosphatase S (PTPRS) | | |
| P04278 | SHBG | Sex hormone-binding globulin | 0.048629015 | 19 |
| | | (SHBG) | | |
| Q8NBP7 | PCSK9 | Proprotein convertase | 0.076209471 | 20 |
| | | subtilisin/kexin type 9 (PCSK9) | | |
| Q93099 | HGD | Homogentisate 1,2-dioxygenase | −0.045385705 | 21 |
| | | (HGD) | | |

n certain embodiments, quantifying concentrations comprises measuring a panel of proteins selected from those identified as MASLD-associated proteins in Tables 1 and 5. The panel can include as few as two proteins or up to all proteins listed in the referenced tables. Representative panels may include any number of proteins within these ranges, for example 2 to 42 proteins. In some embodiments the panel comprises 2, 3, 4, 5, 6, 7, 8, 9, 10, 11, 12, 13, 14, 15, 16, 17, 18, 19, 20, or 21, of the proteins selected from the group consisting of DLD, CFD, SMPDL3A, APOA5, HEXB, GUSB, ADH1A, UGDH, CHMP1A, AKR1C4, RBP5, IHH, STARD10, GLUD1, NRP1, IL1RN, MLN, PTPRS, SHBG, PCSK9, and HGD, or the group consisting of ANGPT2, ANGPTL3, CES1, CHMP1A, CSTZ, DNAJB1, FCGR3B, GLO1, GSTA1, GUSB, IGFBP3, IL1RN, MLN, NRP1, PCSK9, PTPRS, RBP5, RELT, SEMA4D, SMPD1, and SORD.

In certain embodiments, the selection of proteins for a given panel is based on ranking by absolute value of the coefficient, biological plausibility, and/or technical feasibility for targeted proteomic analysis.

In certain embodiments, the biomarkers identified herein are proteins. These proteins are present in plasma or serum and can be quantified using proteomic techniques known in the art, such as mass spectrometry, immunoassays, or automated proteomic analysis systems. Such approaches are consistent with the disclosed workflows for biomarker-based assessment of MASLD.

In certain embodiments, as an alternative to or in addition to quantifying concentrations of the identified proteins, assessment of MASLD status or risk can comprise quantifying gene expression products (e.g., mRNA transcripts) that encode the identified proteins. Such quantification can be performed using nucleic acid amplification or sequencing techniques known in the art, including but not limited to quantitative PCR, digital PCR, or next-generation sequencing. These embodiments provide flexibility for implementing biomarker-based assessment without limiting the approach to proteomic analysis.

In some embodiments, proteomic and transcriptomic data may be integrated to improve predictive performance of MASLD risk scoring algorithms. For example, concentrations of circulating proteins may be combined with expression levels of corresponding mRNA transcripts in a multivariable model to generate a composite MASLD risk score. This approach leverages complementary biological information and aligns with emerging multi-omics strategies for disease classification.

The presently disclosed subject matter includes methods for diagnosing or predicting metabolic dysfunction-associated steatotic liver disease (MASLD) or progression thereof in a subject, and methods of assessing hepatic steatosis in a subject. In certain embodiments, the method comprises obtaining a plasma or serum sample from the subject, quantifying concentrations of a plurality of biomarkers associated with MASLD, and generating a MASLD risk score or other classification metric based on the quantified concentrations. The MASLD risk score or classification metric can be compared to a predetermined threshold to identify the subject as having MASLD, hepatic steatosis, or an increased risk thereof. These methods provide a clinically actionable approach for early detection and risk stratification of MASLD using minimally invasive sampling and computational analysis.

In certain embodiments, obtaining the plasma or serum sample comprises processing whole blood to isolate plasma or serum and performing protein denaturation and/or enzymatic digestion prior to biomarker quantification. Processing whole blood can include centrifugation or other fractionation techniques to separate plasma or serum from cellular components. Protein denaturation may be performed using heat, chemical agents (e.g., urea, guanidine hydrochloride), or detergents to disrupt secondary and tertiary structures, thereby facilitating enzymatic digestion. Enzymatic digestion can be carried out using proteolytic enzymes such as trypsin, Lys-C, or other enzymes commonly employed in targeted proteomics workflows. These steps prepare the sample for accurate quantification of MASLD-associated biomarkers using mass spectrometry or other proteomic techniques, as disclosed herein.

In certain embodiments, quantifying concentrations of the plurality of biomarkers comprises performing a multiplexed mass spectrometry assay configured to detect multiple biomarkers in a single analytical run. Multiplexing can be achieved using isobaric labeling reagents such as tandem mass tags (TMT) or equivalent strategies, which enable simultaneous quantification of multiple proteins across samples. These approaches are widely used in targeted proteomics workflows to improve throughput and analytical precision. In some embodiments, the assay includes liquid chromatography coupled to tandem mass spectrometry (LC-MS/MS) for separation and detection of peptides generated from the biomarkers of interest. Such multiplexed workflows are consistent with the disclosed proteomic methods for MASLD biomarker analysis.

In certain embodiments, quantifying concentrations of the plurality of biomarkers is performed using an automated analytical device configured with a calibration curve tailored to MASLD biomarker ranges. Automated analytical devices can include immunoassay platforms, automated liquid handling systems, or mass spectrometry-based proteomic analyzers designed for high-throughput biomarker quantification. A calibration curve tailored to MASLD biomarker ranges refers to a calibration function generated using reference standards or controls that reflect clinically relevant concentration ranges for MASLD-associated biomarkers, thereby improving accuracy and reproducibility of measurements. These embodiments enable streamlined workflows and reduce operator variability in biomarker quantification.

In certain embodiments, the MASLD risk score generated from biomarker quantification can be validated using a liver-on-a-chip system configured to model hepatic steatosis under controlled conditions. A liver-on-a-chip system refers to a microfluidic or organ-on-chip platform that replicates liver tissue architecture and function, enabling simulation of hepatic fat accumulation and metabolic dysfunction. Such systems can incorporate hepatocytes and supporting cell types within engineered microenvironments, perfusion systems, and nutrient gradients to mimic physiological conditions. Validation using liver-on-a-chip systems provides an experimental approach to confirm MASLD risk predictions and supports translational research for therapeutic development.

In certain embodiments, generating the MASLD risk score comprises applying a multivariable model that assigns biomarker-specific coefficients derived from regression analysis or machine learning algorithms trained on MASLD phenotypes. Regression-based approaches can include linear regression, logistic regression, or penalized regression methods such as LASSO or ridge regression, which estimate relationships between biomarker concentrations and MASLD status. In other embodiments, machine learning algorithms such as gradient boosting, random forest, or neural network models can be employed to capture nonlinear relationships and interactions among biomarkers and clinical covariates. These models can be trained using large-scale datasets and validated using performance metrics such as receiver operating characteristic (ROC) analysis to ensure predictive accuracy. Incorporating these algorithmic strategies enables robust risk scoring and classification for MASLD.

In certain embodiments, generating the MASLD risk score further comprises integrating clinical covariates such as age, body mass index (BMI), and fasting glucose level into the multivariable model. Incorporating these covariates alongside biomarker concentrations improves predictive accuracy by accounting for subject-specific metabolic factors known to influence MASLD risk. These variables can be included as additional independent inputs in regression-based models or machine learning algorithms, enabling a more comprehensive assessment of disease status.

In some embodiments, generating the MASLD risk score comprises applying a multivariable model that assigns biomarker-specific coefficients derived from regression analysis or machine learning algorithms trained on MASLD phenotypes. Regression-based approaches can include linear or logistic regression, while machine learning methods such as gradient boosting, random forest, or neural networks can capture complex interactions among biomarkers and clinical covariates. These models can be trained using large-scale datasets and validated using performance metrics such as receiver operating characteristic (ROC) analysis to ensure robust classification.

In certain embodiments, generating the MASLD risk score is performed by a computing device executing instructions stored on a non-transitory computer-readable medium. The computing device can include servers, desktop computers, laptops, or cloud-based systems configured to process biomarker concentration data and apply predictive algorithms. The instructions can implement regression-based or machine learning models, integrate clinical covariates, and output the MASLD risk score along with classification status.

In some embodiments, the threshold value used to classify MASLD status or risk is determined using receiver operating characteristic (ROC) analysis. ROC analysis evaluates model performance by plotting sensitivity versus false positive rate (1−specificity) across a range of thresholds, enabling selection of a cutoff that optimizes sensitivity and specificity. The area under the ROC curve (AUC) can be used as a measure of overall predictive accuracy, with higher AUC values indicating superior model performance.

In certain embodiments, the method further comprises administering to the subject a therapeutic agent or intervention for treating MASLD when the MASLD risk score exceeds the threshold value. Representative pharmacologic agents include sodium-glucose cotransporter 2 (SGLT2) inhibitors (e.g., empagliflozin), glucagon-like peptide-1 (GLP-1) receptor agonists (e.g., semaglutide), dual glucose-dependent insulinotropic polypeptide and GLP-1 receptor agonists (e.g., tirzepatide), thiazolidinediones (TZDs) (e.g., pioglitazone), biguanides (e.g., metformin), thyroid hormone receptor beta (THR-β) agonists (e.g., resmetirom), fibroblast growth factor (FGF) analogs (e.g., pegbelfermin), farnesoid X receptor (FXR) agonists (e.g., obeticholic acid), peroxisome proliferator-activated receptor (PPAR) agonists (e.g., lanifibranor), acetyl-coenzyme A carboxylase (ACC) inhibitors (e.g., firsocostat), diacylglycerol O-acyltransferase 2 (DGAT2) inhibitors (e.g., vupanorsen), C—C chemokine receptor type 2 and type 5 (CCR2/CCR5) antagonists (e.g., cenicriviroc), antioxidants (e.g., vitamin E), and statins (e.g., rosuvastatin). In certain embodiments, the therapeutic agent will alter abundance of one or more MASLD associated biomarkers. In addition to pharmacologic therapy, clinical management may include bariatric surgery and structured lifestyle interventions such as diet modification and exercise programs. In certain embodiments, the method further comprises additional testing of said subject for coronary risk, such as coronary calcification, echocardiography, catheterization, stress testing.

The presently disclosed subject matter includes a kit for diagnosing or predicting MASLD in a subject. The kit comprises a plurality of reagents configured for detection of MASLD-associated biomarkers, such as those listed in Tables 1 and 5. These reagents can be provided in formats suitable for multiplexed analysis, including immunoassay cartridges, microfluidic chips, or mass spectrometry-compatible consumables. The kit further includes instructions for calculating a MASLD risk score based on quantified biomarker concentrations. The instructions may be provided in printed form or stored on a non-transitory computer-readable medium and can implement algorithms such as regression-based models or machine learning approaches trained on MASLD phenotypes. In some embodiments, the kit may also include calibration standards and quality control materials to ensure accurate biomarker quantification.

In certain embodiments, the reagents are provided in a multiplexed assay cartridge configured for simultaneous detection of two or more MASLD-associated biomarkers in a single analytical run. Multiplexed cartridges can include microfluidic channels or wells preloaded with capture reagents, enabling high-throughput and efficient biomarker quantification. In some embodiments, the multiplexed assay cartridge comprises a calibration curve tailored to MASLD biomarker ranges. This calibration curve is generated using reference standards that reflect clinically relevant concentration ranges for MASLD-associated proteins, ensuring accurate quantification across the dynamic range observed in MASLD patients.

In certain embodiments, the reagents comprise antibody pairs configured for use in a sandwich immunoassay for detecting MASLD biomarkers. Sandwich immunoassays provide high specificity and sensitivity by capturing the target biomarker between two antibodies, one immobilized and one labeled for detection.

In some embodiments, the instructions stored on a non-transitory computer-readable medium comprise executable code for applying a multivariable model that calculates a MASLD risk score. The code may implement regression-based algorithms or machine learning models trained on MASLD phenotypes and validated using ROC analysis.

In certain embodiments, the instructions further comprise code for comparing the MASLD risk score to a threshold value determined using ROC analysis. This enables classification of the subject as MASLD-positive or at increased risk based on statistically validated cutoffs.

In some embodiments, the kit further comprises an automated immunoassay device configured to process the multiplexed assay cartridge and transmit biomarker concentration data to a computing device for MASLD risk score calculation.

In certain embodiments, the instructions further comprise code for integrating clinical covariates such as age, BMI, and fasting glucose level into the MASLD risk score calculation, improving predictive accuracy.

The presently disclosed subject matter includes computer-implemented methods for diagnosing or predicting MASLD risk in a subject. In certain embodiments, the method comprises receiving as input quantified concentrations of MASLD-associated biomarkers, such as those listed in Tables 1 and 5. The method applies a multivariable model trained on MASLD phenotypes to these concentrations. The model may include regression-based algorithms (e.g., linear or logistic regression, LASSO) or machine learning approaches (e.g., gradient boosting, random forest, neural networks) to calculate a MASLD risk score. The MASLD risk score is then output via a graphical user interface or electronic report, enabling clinical interpretation and decision-making. In some embodiments, the computing device may be a local workstation or a cloud-based server configured to execute instructions stored on a non-transitory computer-readable medium.

In certain embodiments, the computing device comprises a processor configured to execute instructions stored on a non-transitory computer-readable medium to apply the multivariable model. The processor may be part of a local workstation or a cloud-based server, enabling scalable computation for MASLD risk scoring.

In some embodiments, applying the multivariable model comprises executing a trained machine learning algorithm such as gradient boosting regression, random forest regression, or neural network regression. These algorithms can capture nonlinear relationships among biomarkers and clinical covariates, improving predictive accuracy.

In certain embodiments, generating the MASLD risk score further comprises comparing the score to a threshold value determined using ROC analysis. ROC analysis evaluates sensitivity and specificity across thresholds, enabling selection of a cutoff that optimizes classification performance.

In some embodiments, the multivariable model integrates clinical covariates such as age, BMI, and fasting glucose level into the MASLD risk score calculation, providing a more comprehensive assessment of disease risk.

In certain embodiments, applying the multivariable model further comprises normalizing biomarker concentrations using a calibration curve tailored to MASLD biomarker ranges, ensuring accurate interpretation of proteomic data.

In some embodiments, outputting the MASLD risk score comprises generating a graphical user interface that displays the MASLD risk score and classification status, enabling clinicians to interpret results easily.

In certain embodiments, the computing device comprises a cloud-based server configured to receive biomarker concentration data from a remote assay device and execute the multivariable model, supporting distributed workflows and telehealth applications.

The presently disclosed subject matter includes methods for establishing a role for a gene target in MASLD or progression thereof. In certain embodiments, the method comprises assessing a MASLD phenotype in a plurality of subjects and correlating said phenotype with gene expression patterns indicative of MASLD pathophysiology. These approaches enable identification of gene targets that contribute to disease onset or progression.

In some embodiments, assessing comprises obtaining circulating proteomic measures or transcriptomic data, including single-cell or bulk RNA sequencing, for one or more gene targets. These data types provide complementary insights into protein abundance and gene expression, supporting mechanistic understanding of MASLD.

In certain embodiments, the method further comprises deconvolution of assessed gene expression to resolve cell-type-specific contributions. Deconvolution algorithms can be applied to bulk transcriptomic data to infer expression profiles of hepatocytes, stellate cells, and immune cell populations relevant to MASLD.

In some embodiments, the one or more gene targets are selected from those listed in Table 1 or Table 5, which include genes encoding MASLD-associated proteins identified through large-scale proteomic and transcriptomic analyses.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUBMB Joint Commission on Biochemical Nomenclature (See, iubmb.qmul.ac.uk/).

Although any methods, devices, and materials similar or equivalent to those described herein can be used in the practice or testing of the presently-disclosed subject matter, representative methods, devices, and materials are described herein.

In certain instances, nucleotides and polypeptides disclosed herein are included in publicly-available databases, such as NCBI® Gene (also known as Entrez Gene), GENBANK® and UNIPROT®. Information including sequences and other information related to such nucleotides and polypeptides included in such publicly available databases are expressly incorporated by reference. Unless otherwise indicated or apparent the references to such publicly available databases are references to the most recent version of the database as of the filing date of this Application.

Following long-standing patent law convention, the terms “a”, “an”, and “the” refer to “one or more” when used in this application, including the claims. Thus, for example, reference to “a protein” includes a plurality of such proteins, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, in some embodiments ±0.1%, in some embodiments ±0.01%, and in some embodiments ±0.001% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the term “automated analytical device” refers to an instrument configured to perform biomarker quantification with minimal manual intervention, including but not limited to immunoassay platforms, automated liquid handling systems, or mass spectrometry-based proteomic analyzers.

As used herein, the term “biological sample” refers to any sample derived from a subject that contains biomarkers of interest, including but not limited to blood, plasma, serum, or other bodily fluids. As used herein, the term “plasma” refers to the liquid component of blood obtained after centrifugation of whole blood treated with an anticoagulant, and the term “serum” refers to the liquid component obtained after clotting and centrifugation of whole blood without anticoagulant.

As used herein, the term “biomarker-specific coefficients” refers to numerical weights assigned to individual biomarkers within a predictive model, derived from statistical or machine learning training on MASLD phenotypes.

As used herein, the term “calibration curve tailored for MASLD biomarker ranges” refers to a calibration function generated using reference standards or controls that reflect the expected concentration ranges of MASLD-associated biomarkers, enabling accurate quantification within clinically relevant limits.

As used herein, the term “clinical covariates” refers to subject-specific variables that provide additional context for disease risk assessment, including but not limited to age, body mass index (BMI), and fasting glucose level.

The present application can “comprise” (open ended) or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

As used herein, “computer-implemented method” refers to a process executed by one or more processors configured to perform the disclosed steps using machine-readable instructions stored on a non-transitory computer-readable medium.

As used herein, the term “computing device” refers to any hardware system capable of executing instructions stored on a computer-readable medium, including but not limited to servers, desktop computers, laptops, tablets, or cloud-based systems.

As used herein, the term “enzymatic digestion” refers to the cleavage of proteins into peptides using proteolytic enzymes.

As used herein, the term “executable code” refers to computer instructions stored on a non-transitory medium that can be executed by a processor to perform specific functions, such as MASLD risk score calculation.

As used herein, the term “generating,” when used in connection with a MASLD risk score, refers to calculating or producing the MASLD risk score by applying a mathematical or algorithmic model to biomarker concentration data, optionally integrating clinical covariates.

As used herein, “graphical user interface” refers to a visual display environment that presents calculated results (e.g., MASLD risk score) in a human-readable format and optionally provides interpretive categories, alerts, and recommendations.

As used herein, the term “identifying,” when used in connection with a subject, refers to classifying a subject as having MASLD or an increased risk thereof by comparing the MASLD risk score to a predetermined threshold value.

As used herein, the term “intervention” refers to any non-pharmacologic treatment strategy, including but not limited to dietary modification, exercise programs, or surgical procedures such as bariatric surgery.

As used herein, the term “machine learning algorithm” refers to a computational method that learns patterns from data to make predictions, including but not limited to gradient boosting, random forest, or neural network models.

As used herein, the term “metabolic dysfunction-associated steatotic liver disease” or “MASLD” refers to a liver condition characterized by hepatic steatosis, defined as excess fat accumulation in the liver in the presence of one or more metabolic risk factors, including but not limited to obesity, insulin resistance, dyslipidemia, or type 2 diabetes. MASLD encompasses disease stages ranging from simple steatosis to advanced fibrosis and cirrhosis. Excess fat accumulation refers to an amount of fat in the liver that exceeds normal physiological levels and, in certain embodiments, is defined as fat present in at least about 5% of hepatocytes, as determined by histological examination of liver tissue. In other embodiments, excess fat accumulation corresponds to a liver fat content of at least about 5% by weight, as assessed by imaging modalities such as magnetic resonance imaging-proton density fat fraction (MRI-PDFF) or other equivalent quantitative techniques. These thresholds are consistent with clinical and research standards for diagnosing hepatic steatosis.

As used herein, the term “MASLD risk score” refers to a numerical value indicative of MASLD status or risk, calculated based on biomarker concentration data. In certain embodiments, the MASLD risk score is generated by applying a multivariable model, which comprises biomarker-specific coefficients derived from regression analysis or machine learning algorithms trained on MASLD phenotypes. The MASLD risk score may optionally integrate clinical covariates such as age, body mass index, and fasting glucose level, and may be validated using statistical performance metrics such as receiver operating characteristic (ROC) curves.

As used herein, the term “multivariable model” refers to a mathematical or computational model that uses two or more independent variables (e.g., biomarker concentrations, clinical covariates) to generate a predictive output, such as a MASLD risk score.

As used herein, the term “non-transitory computer-readable medium” refers to a physical storage medium that retains data and executable instructions, such as hard drives, solid-state drives, optical discs, or flash memory, and explicitly excludes transitory signals.

As used herein, “optional” or “optionally” means that the subsequently described event or circumstance does or does not occur and that the description includes instances where said event or circumstance occurs and instances where it does not.

As used herein, the term “plurality of biomarkers” refers to two or more analytes whose concentrations in a biological sample, such as plasma or serum, are associated with MASLD status or progression.

As used herein, the term “processing whole blood” refers to any procedure that separates plasma or serum from cellular components, including but not limited to centrifugation, filtration, or other fractionation techniques commonly used in clinical and research settings.

As used herein, the term “protein denaturation” refers to the disruption of secondary and tertiary protein structures to expose peptide bonds for enzymatic digestion, typically achieved using heat, chemical agents (e.g., urea, guanidine hydrochloride), or detergents.

As used herein, the term “quantifying concentrations” refers to measuring the amount of each biomarker in a biological sample using analytical techniques known in the art, including but not limited to mass spectrometry-based proteomics, immunoassays, or automated proteomic analysis systems. Such quantification may include sample preparation steps such as protein digestion, denaturation, and calibration.

As used herein, the term “receiver operating characteristic (ROC) analysis” refers to a performance evaluation method for a predictive model that plots the true positive rate (sensitivity) on the Y-axis against the false positive rate, which is calculated as 1−specificity, on the X-axis across a range of decision thresholds. This analysis illustrates the trade-off between correctly identifying positive cases and incorrectly classifying negative cases and is commonly used to assess the discriminatory ability of diagnostic and predictive algorithms. The area under the ROC curve (AUC) provides a quantitative measure of overall model performance, with values closer to 1.0 indicating superior accuracy.

As used herein, the term “regression analysis” refers to a statistical modeling technique that estimates relationships between dependent and independent variables, including linear regression, logistic regression, or other regression-based approaches commonly used in biomedical data analysis.

As used herein, “risk” refers to the probability or likelihood that a subject will develop MASLD or experience progression of MASLD within a defined time horizon, as estimated by comparing the subject's MASLD risk score to a reference distribution or by applying a predictive model trained on empirical outcome data. As will be appreciated by one of ordinary skills in the art, risk prediction does not imply certainty or guarantee of future outcomes; rather, it provides a probabilistic estimate based on population-level associations and statistical modeling. Such estimates inherently involve variability and uncertainty and are intended to inform clinical decision-making rather than serve as an absolute determinant of disease occurrence.

As used herein, the term “subject” refers to any mammalian individual for whom assessment of hepatic steatosis or risk thereof is desired. In certain embodiments, the subject is a human. In other embodiments, the subject is a non-human mammal, including but not limited to companion animals (e.g., dogs, cats), livestock (e.g., horses, cattle), or research animals (e.g., rodents, primates). The term encompasses healthy individuals as well as those with existing or suspected hepatic steatosis, including metabolic dysfunction-associated steatotic liver disease, or related conditions.

As used herein, the term “therapeutic agent” refers to any pharmacologic compound, biologic, or intervention administered to a subject to treat, manage, or reduce the risk of MASLD or its progression.

As used herein, the term “threshold value” refers to a predetermined cutoff used to classify a subject as MASLD-positive or at increased risk thereof. The threshold value may be derived from a ROC curve or other statistical method to optimize sensitivity and specificity for MASLD classification.

The presently disclosed subject matter is further illustrated by the following specific but non-limiting examples. The following examples may include compilations of data that are representative of data gathered at various times during the course of development and experimentation related to the present invention.

### EXAMPLES

### Example 1: General Flow of Study and Characteristics of Study Samples

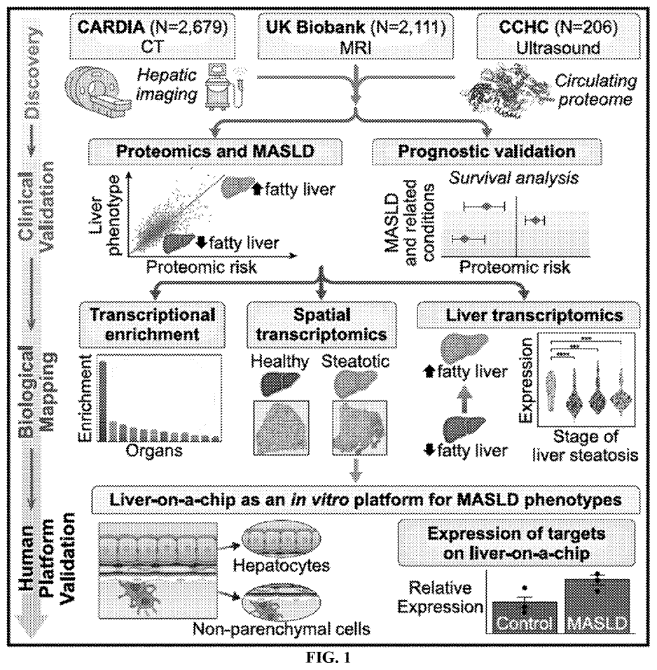

The study consisted of six integrated steps, specifically (1) identification and validation of a “hepatic steatosis” proteome (across 4996 participants across three prospective observational studies: Coronary Artery Risk Development in Young Adults [CARDIA], UK Biobank, Cameron County Hispanic Cohort [CCHC]); (2) relation of this proteome to clinical outcomes relevant to hepatic steatosis (across 26421 participants in UK Biobank over a median 13.7 years of follow-up); (3) characterization of tissue origin and implicated molecular pathways; (4) examining expression of genes encoding the hepatic steatosis proteome in human liver across the complete MASLD activity and stage spectrum (bulk RNA-seq in human liver; SteatoSITE, N=499 biopsies, 74 with end-stage F4 fibrosis); (5) specifying cell and spatially resolved expression of these genes in human liver with and without MASLD (scRNA-seq, N=19; spatial transcriptomics, N=4); (6) testing targets on a humanized “liver-on-a-chip” platform to examine transcriptional changes with induction of steatosis and whether these translated to protein elaboration. Additional reference is made to FIG. 1 and the methods set forth in the Examples below.

The characteristics of the study samples are shown in Tables 2-4.

TABLE 2
Baseline characteristics of CARDIA study population.

| | Overall | Derivation | Validation | |
|---|---|---|---|---|
| Characteristic | N = 2,679 | N = 1,876 | N = 803 | P-value |
| Age | 51.0 (47.0, 53.0); | 50.0 (47.0, 53.0); | 51.0 (47.0, 53.0); | 0.2 |
| | 0% | 0% | 0% | |
| Gender | | | | 0.3 |
| Male | 1,149 (43%); 0% | 793 (42%); 0% | 356 (44%); 0% | |
| Female | 1,530 (57%); 0% | 1,083 (58%); 0% | 447 (56%); 0% | |
| Race | | | | >0.9 |
| Black | 1,259 (47%); 0% | 882 (47%); 0% | 377 (47%); 0% | |
| White | 1,420 (53%); 0% | 994 (53%); 0% | 426 (53%); 0% | |
| CARDIA field center | | | | 0.5 |
| Birmingham, AL | 632 (24%); 0% | 433 (23%); 0% | 199 (25%); 0% | |
| Chicago, IL | 623 (23%); 0% | 435 (23%); 0% | 188 (23%); 0% | |
| Minneapolis, MN | 707 (26%); 0% | 491 (26%); 0% | 216 (27%); 0% | |
| Oakland, CA | 717 (27%); 0% | 517 (28%); 0% | 200 (25%); 0% | |
| BMI (kg/m²) | 29 (25, 34); 0% | 29 (25, 34); 0% | 29 (25, 34); 0% | 0.5 |
| Drinks/week | 1.0 (0.0, 5.0); 0% | 1.0 (0.0, 5.0); 0% | 1.0 (0.0, 5.0); 0% | 0.8 |
| Mean systolic blood pressure | 117 (108, 127); | 117 (108, 127); | 117 (108, 127); | 0.6 |
| (mmHg) | 0.1% | 0.1% | 0.1% | |
| Mean diastolic blood pressure | 73 (66, 80); 0.1% | 73 (66, 80); 0.2% | 73 (66, 81); 0.1% | >0.9 |
| (mmHg) | | | | |
| On anti-hypertensive therapy | 724 (27%); 0% | 504 (27%); 0% | 220 (27%); 0% | 0.8 |
| On cholesterol lowering | 438 (16%); 0% | 312 (17%); 0% | 126 (16%); 0% | 0.5 |
| medication | | | | |
| Diabetes | 395 (15%); 0% | 280 (15%); 0% | 115 (14%); 0% | 0.7 |
| Lifetime pack-years smoking | 0 (0, 6); 0% | 0 (0, 6); 0% | 0 (0, 7); 0% | 0.9 |
| Total cholesterol (mg/dL) | 190 (166, 215); 0% | 190 (166, 215); 0% | 189 (167, 212); 0% | 0.9 |
| High-density lipoprotein (mg/dL) | 55 (45, 67); 0% | 54 (45, 67); 0% | 55 (44, 66); 0% | 0.6 |
| eGFR (ml/min/1.73 m²) | 94 (82, 108); <0.1% | 94 (82, 108); 0% | 95 (82, 109); 0.2% | 0.2 |
| Hemoglobin A1c (%) | 5.50 (5.30, 5.80); | 5.50 (5.30, 5.80); | 5.50 (5.30, 5.80); | >0.9 |
| | 1.2% | 1.1% | 1.2% | |
| Sum of AHA Life Simple 7 | 9.00 (7.00, 10.00) | 9.00 (7.00, 10.00); | 9.00 (7.00, 10.00); | 0.5 |
| | ; 21% | 21% | 23% | |
| Visceral adipose tissue volume | 118 (77, 171); 0.7% | 119 (78, 171); 0.6% | 116 (74, 174); 0.7% | 0.6 |
| (cm³) | | | | |
| Subcutaneous adipose tissue | 308 (212, 446); | 310 (214, 447); | 301 (204, 443); | 0.2 |
| volume (cm³) | 0.7% | 0.6% | 0.7% | |
| Mean liver attenuation (Hounsfield | 58 (51, 62); 0% | 58 (51, 62); 0% | 58 (51, 62); 0% | 0.8 |
| units, HU) | | | | |
| MASLD (defined as liver | 268 (10%); 0% | 183 (9.8%); 0% | 85 (11%); 0% | 0.5 |
| attenuation <40 HU) | | | | |

Continuous measures are reported as median (25^{th} percentile, 75^{th} percentile). Categorical measures are reported as n (%). Percent missingness is reported after the semi-colon for each cell. P-values are from Wilcoxon rank sum tests for continuous measures and chi square tests for categorical measures comparing the derivation and validation subsamples.

TABLE 3
Baseline characteristics of UK Biobank participants.

| | | | Missing liver MRI | | Liver MRI data | | |
|---|---|---|---|---|---|---|---|
| | Overall | | data | | present | | |
| Characteristic | N = 26,421 | | N = 24,310 | | N = 2,111 | | P-value |
| Age | 58 | (50, 64) | 59 | (51, 64) | 54 | (47, 60) | <0.001 |
| Female | 14,242 | (54%) | 13,128 | (54%) | 1,114 | (53%) | 0.3 |
| Race | | | | | | | <0.001 |
| Asian | 534 | (2.0%) | 512 | (2.1%) | 22 | (1.0%) | |
| Black | 564 | (2.1%) | 545 | (2.2%) | 19 | (0.9%) | |
| Mixed | 177 | (0.7%) | 161 | (0.7%) | 16 | (0.8%) | |
| Unknown-other | 432 | (1.6%) | 411 | (1.7%) | 21 | (1.0%) | |
| White | 24,714 | (94%) | 22,681 | (93%) | 2,033 | (96%) | |
| Body mass index (kg/m²) | 26.8 | (24.2, 29.9) | 26.9 | (24.3, 30.0) | 25.9 | (23.6, 28.6) | <0.001 |
| Unknown | 121 | | 119 | | 2 | | |
| Systolic blood pressure (mmHg) | 138 | (125, 152) | 138 | (126, 152) | 134 | (122, 147) | <0.001 |
| Unknown | 1,609 | | 1,446 | | 163 | | |
| Diabetes | 1,540 | (5.8%) | 1,497 | (6.2%) | 43 | (2.0%) | <0.001 |
| Unknown | 28 | | 28 | | 0 | | |
| Townsend Deprivation Index | −2.1 | (−3.6, 0.7) | −2.0 | (−3.6, 0.8) | −2.6 | (−3.9, −0.1) | <0.001 |
| Unknown | 35 | | 32 | | 3 | | |
| Smoking | | | | | | | <0.001 |
| Current | 2,814 | (11%) | 2,677 | (11%) | 137 | (6.5%) | |
| Never \| No Answer | 14,350 | (54%) | 13,082 | (54%) | 1,268 | (60%) | |
| Previous | 9,226 | (35%) | 8,520 | (35%) | 706 | (33%) | |
| Unknown | 31 | | 31 | | 0 | | |
| Alcohol frequency | | | | | | | <0.001 |
| Never \| No Answer | 2,290 | (8.7%) | 2,185 | (9.0%) | 105 | (5.0%) | |
| Special occasions only | 3,137 | (12%) | 2,956 | (12%) | 181 | (8.6%) | |
| One to three times a month | 2,817 | (11%) | 2,615 | (11%) | 202 | (9.6%) | |
| Once or twice a week | 6,919 | (26%) | 6,378 | (26%) | 541 | (26%) | |
| Three or four times a week | 5,946 | (23%) | 5,348 | (22%) | 598 | (28%) | |
| Daily or almost daily | 5,281 | (20%) | 4,797 | (20%) | 484 | (23%) | |
| Unknown | 31 | | 31 | | 0 | | |
| Liver proton density fat fraction | 3.0 | (2.2, 5.1) | — | | 3.0 | (2.2, 5.1) | |
| Unknown | 24,310 | | — | | 0 | | |
| Aspartate aminotransferase | 24 | (21, 29) | 24 | (21, 29) | 24 | (21, 28) | <0.001 |
| (U/L) | | | | | | | |
| Unknown | 1,343 | | 1,239 | | 104 | | |
| Alanine aminotransferase (U/L) | 20 | (15, 27) | 20 | (15, 27) | 19 | (15, 26) | <0.001 |
| Unknown | 1,261 | | 1,163 | | 98 | | |
| Hemoglobin A1c (mmol/mol) | 35.3 | (32.8, 38.1) | 35.4 | (32.9, 38.2) | 34.4 | (31.9, 36.8) | <0.001 |
| Unknown | 1,370 | | 1,253 | | 117 | | |
| Low density lipoprotein | 3.48 | (2.91, 4.10) | 3.48 | (2.90, 4.10) | 3.49 | (2.93, 4.08) | 0.8 |
| (mmol/L) | | | | | | | |

| | | | |
|---|---|---|---|
| Unknown | 1,321 | 1,219 | 102 |

Continuous measures are reported as median (25^{th} percentile, 75^{th} percentile). Categorical measures are reported as n (%). P-values are from Wilcoxon rank sum tests for continuous measures and chi square tests for categorical measures comparing participants with and without liver MRI data.

TABLE 4
Baseline characteristics of the Cameron County Hispanic Cohort.

| Characteristic | N | | |
|---|---|---|---|
| Age (years) | 206 | 56 | (46, 66) |
| Female | 206 | 136 | (66%) |
| White Hispanic/Latino | 206 | 206 | (100%) |
| Body mass index (kg/m²) | 206 | 30.9 | (27.4, 35.8) |
| Type 2 Diabetes | 206 | 66 | (32%) |
| Hypertension | 201 | 22 | (11%) |
| Alcohol use | 128 | | |
| Never | | 63 | (49%) |
| Sometimes | | 52 | (41%) |
| Other | | 13 | (10%) |
| Controlled attenuation | 206 | 297 | (247, 337) |
| parameter (dB/m) | | | |

Continuous measures are reported as median (25^{th} percentile, 75^{th} percentile). Categorical measures are reported as n (%). Hypertension defined as systolic blood pressure ≥140 mmHg or diastolic blood pressure ≥90 mmHg.

Overall, the study included 2679 CARDIA study participants after excluding participants with potential secondary (i.e., non-MASLD) causes for hepatic dysfunction or steatosis (>14 alcoholic drinks/week, hepatitis C, cirrhosis, HIV, and use of amiodarone, valproic acid, methotrexate, tamoxifen, or diltiazem)¹⁰. CARDIA participants were randomly split into derivation (N=1876) and validation (N=803) subsamples (balanced by CT liver attenuation), with an overall median age of 51 years, 57% female, and 47% Black individuals (Table 2). Given the focus was on a broad discovery within hepatic steatosis, the population was not restricted to specific MASLD-defining criteria¹¹: nevertheless, the majority of the included CARDIA participants have relevant risk factors that are required for a clinical diagnosis of MASLD (BMI≥25 kg/m² in ≈75%; diabetes in ≈15%; low alcohol use, median 1 drink/week).

UK Biobank and the Cameron County Hispanic cohort (CCHC) served as validation sets for the current study. The study sample in UK Biobank included 26421 participants across a broad age range (25^{th} to 75^{th} percentile: 50-64 years), with 54% women. Participants were predominantly White (94%) and overweight (median 27 kg/m²), with a lower prevalence of diabetes (5.8%) and greater alcohol intake (43% reporting≥3 times a week). A total of 2111 UK Biobank participants had MRI measures of hepatic steatosis (Table 3). CCHC participants (N=206) were all Hispanic or Latino, had similar age (25^{th} to 75^{th} percentile: 46-66 years) and gender (66% female) distributions, with greater BMI (median 31 kg/m²), and diabetes prevalence (32%; Table 4).

### Example 2: Pathway and Tissue Specificity of the Circulating Proteome of MASLD

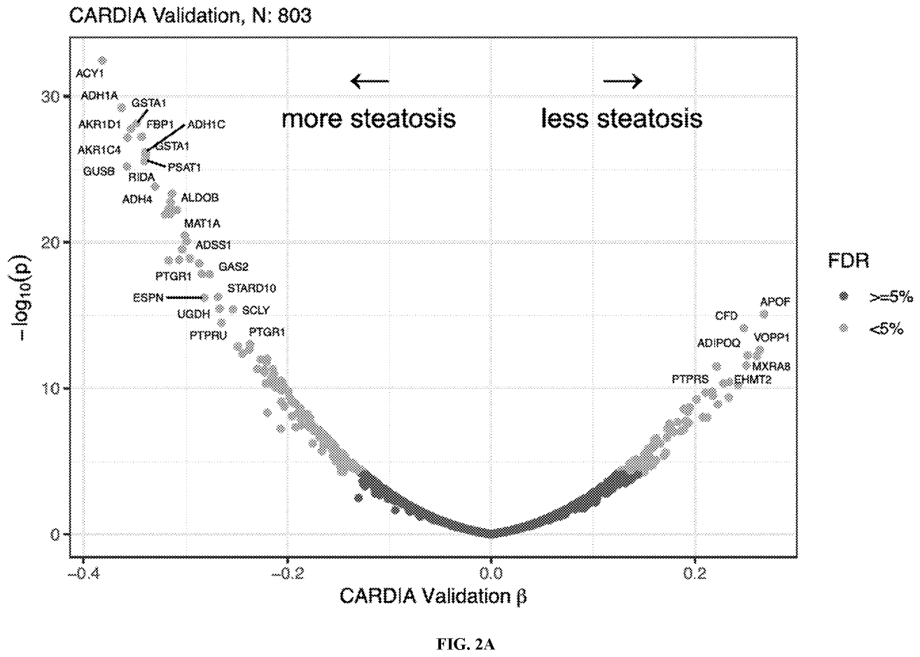

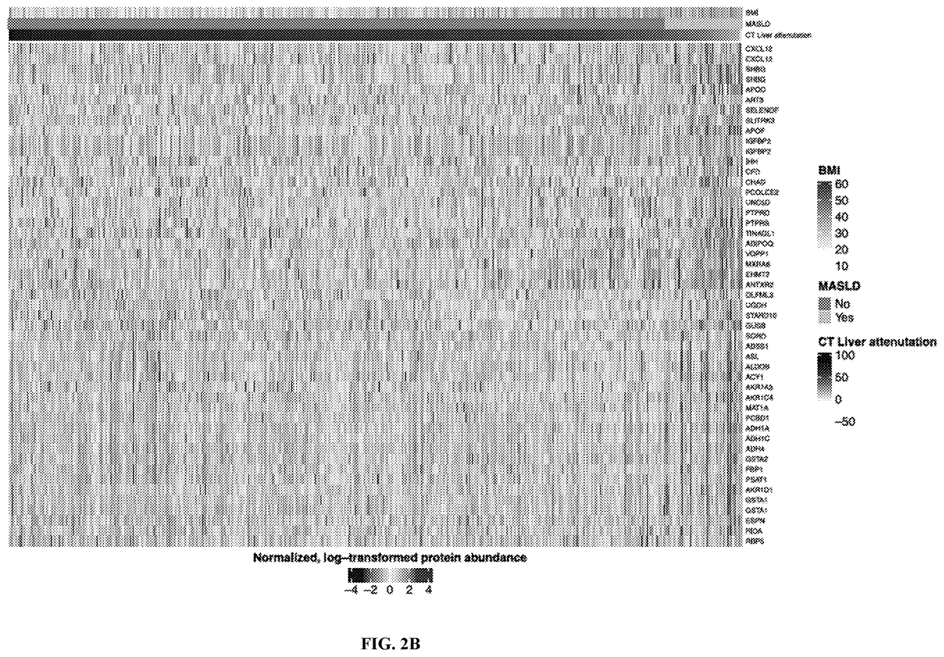

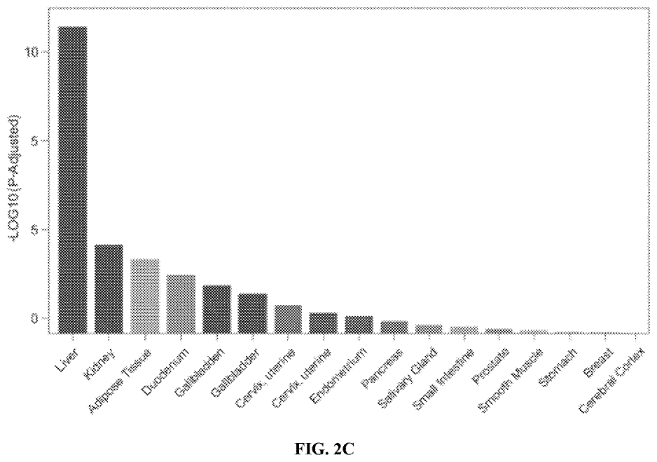

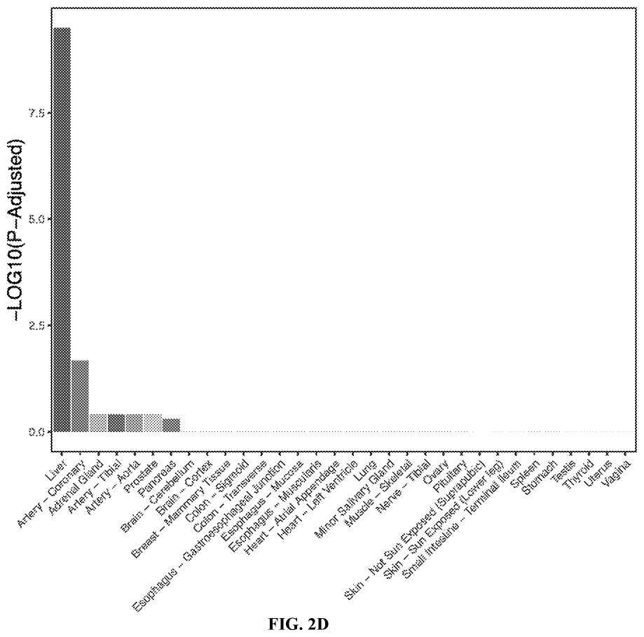

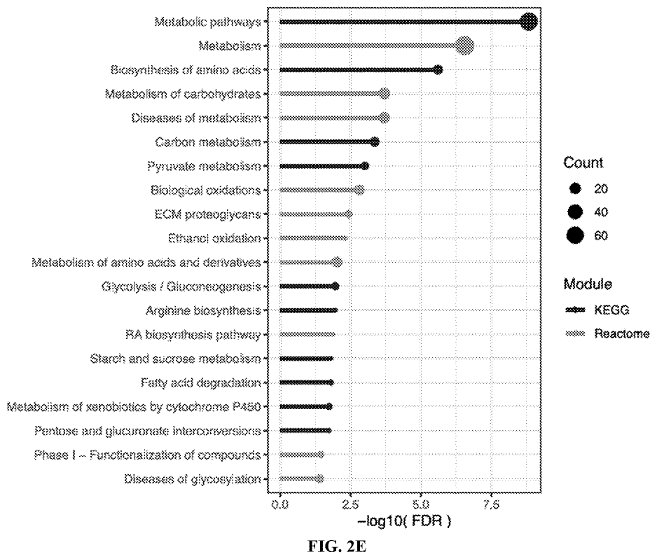

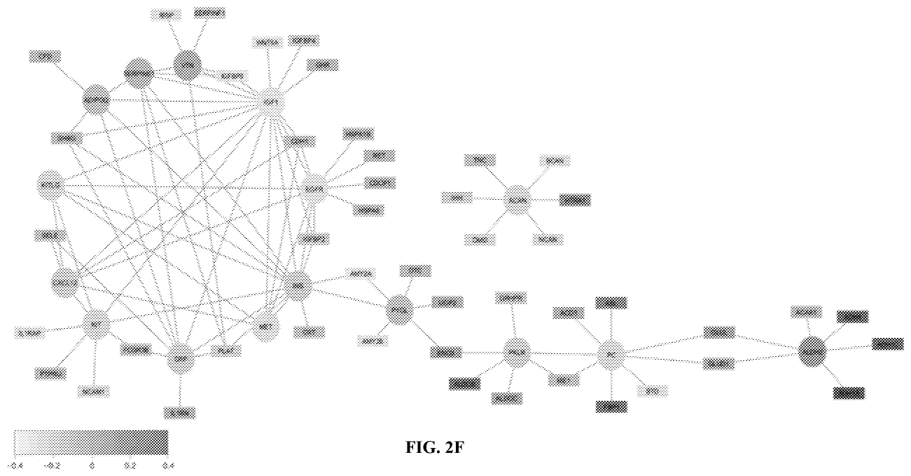

Across 2679 participants in CARDIA with SomaScan 7k proteomics, 237 unique proteins (259 SomaScan aptamers) were identified that are associated with liver attenuation on computed tomography (lower liver attenuation~more hepatic steatosis) across both derivation and validation subsamples (adjusted for age, gender, race, BMI; FIG. 2A-B; full regression estimates were obtained for liver attenuation as a function of individual proteins adjusted for age, gender, race, and BMI in CARDIA (data not shown)).

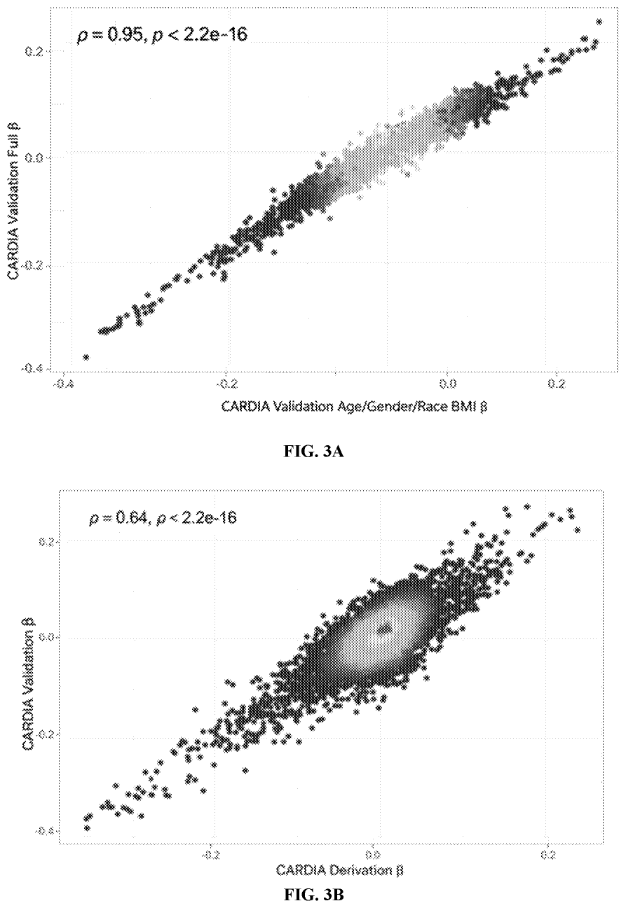

Regression estimates were robust to multivariable adjustment, including metabolic risk factors, renal function, physical activity, and alcoholic drinks per week (relation of regression estimates across adjustments: Spearman ρ=0.95; P<2.2×10⁻¹⁶; FIG. 3A-3B; full regression model results were obtained for liver attenuation as a function of individual proteins adjusted for age, gender, race, BMI, alcoholic drinks per week, systolic blood pressure, use of hypertensive and cholesterol medications, diabetes, pack years of smoking, total cholesterol, high-density lipoprotein, estimated glomerular filtration rate, and moderate-vigorous physical activity in CARDIA (data not shown)).

Significant enrichment of expression was observed for those protein targets from epidemiologic studies in CARDIA at the transcriptional (FIG. 2C) and tissue proteomic level (FIG. 2D), specifying broad pathways implicated in central metabolic processes (e.g., carbon, pyruvate, amino acid, carbohydrate metabolism) and fibrosis (FIG. 2E), including known and emerging mechanisms of MASLD, namely amino acid metabolism (ACY1^{8,12}, FAH¹³), alcohol processing (e.g., ADH1A^{14,15}), fructose catabolism (ALDOB, SORD¹⁶), bile acid and steroid metabolism (AKR1D1¹⁷, AKR1C4^{18,19}), gluconeogenesis (FBP1²⁰), and multi-substrate detoxification, intermediary metabolism, and fibrosis (GSTA1²¹, ASL²², UGDH²³), among others. To identify potential central mediators of MASLD, an interaction (hub gene) analysis was conducted including 235 genes (235 of the 237 unique proteins were present in the STRING database²⁴), with nodes identifying genes with central relevance to MASLD (FIG. 2F). Identified nodes included pathogenic mediators of hepatocyte regeneration and fibrosis regulation (EGFR²⁵, IGF-1²⁶), apoptosis regulation (MET²⁷), inflammatory mediators (CXCL2²⁸, CRP, SERPINE1), extracellular matrix responses to hepatic injury (VTN²⁹, ACAN³⁰), glycogen metabolism (PYGL³¹), and mitochondrial pyruvate metabolism (PKLR³², PC³³), among several other canonical markers of insulin sensitivity and adiposity (ADIPOQ, INS). These results suggested a predominant hepatic origin for the circulating MASLD proteome, implicating canonical metabolic-inflammatory-fibrotic pathways in liver degeneration.

### Example 3: Generation and Replication of a Proteomic Score for Hepatic Steatosis

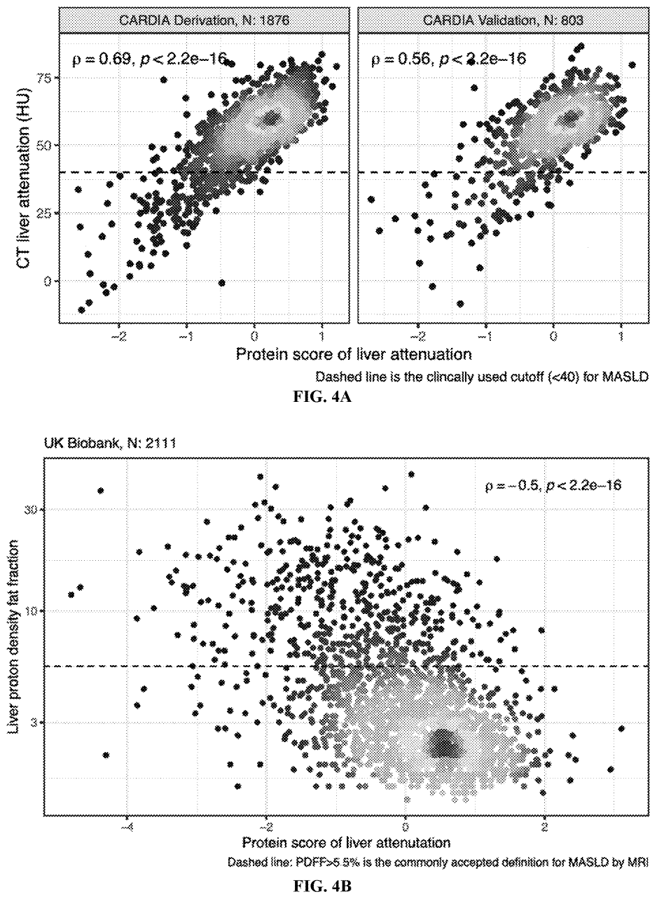

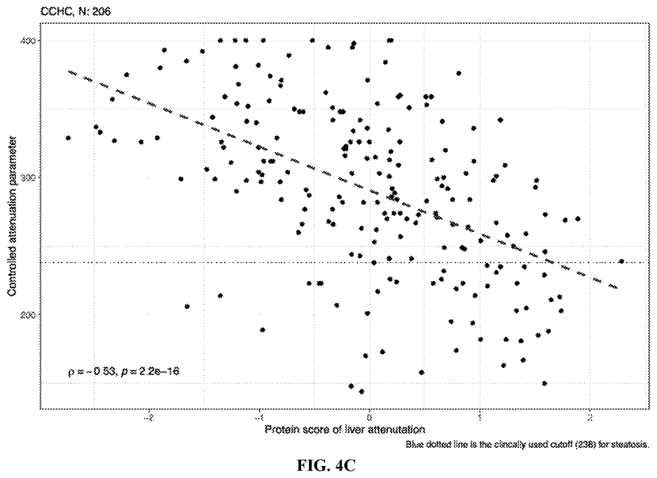

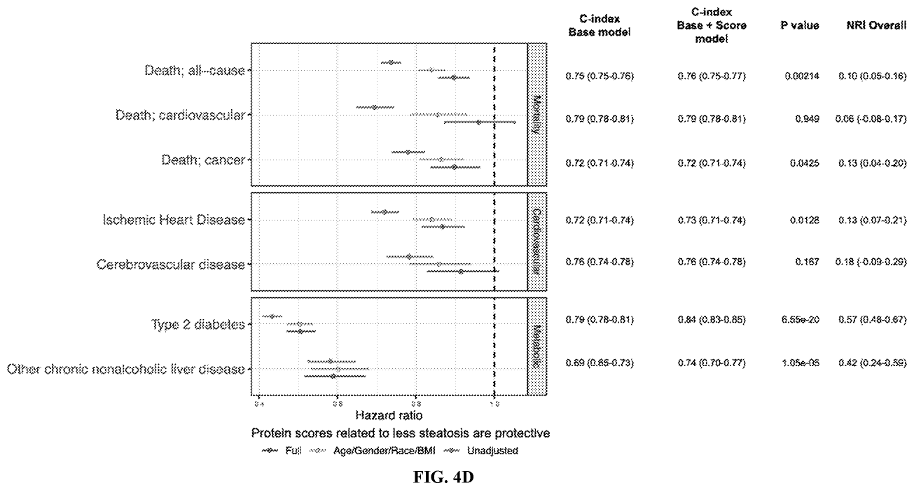

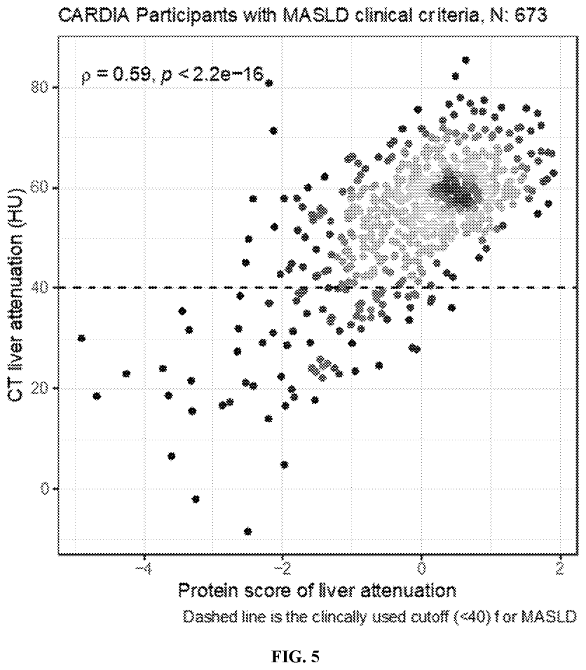

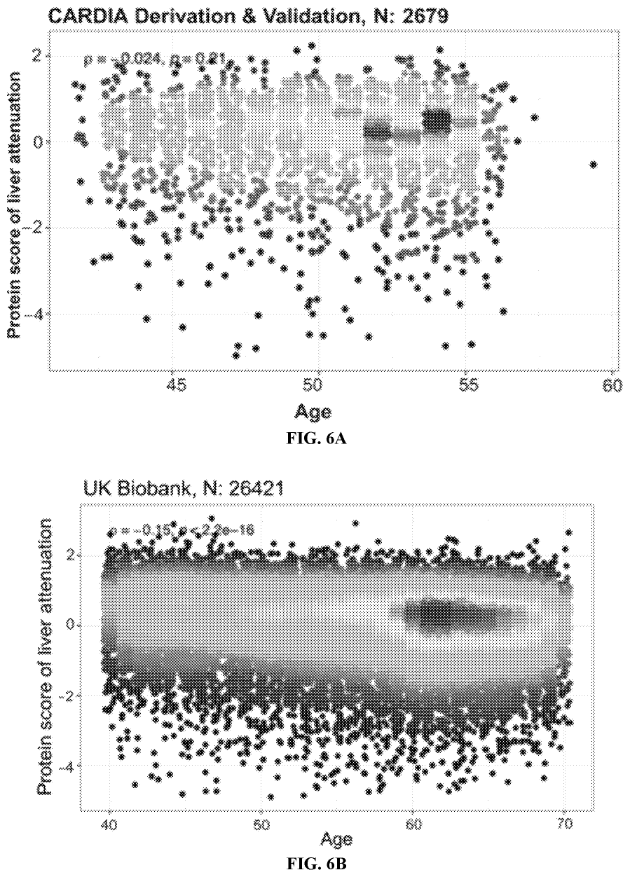

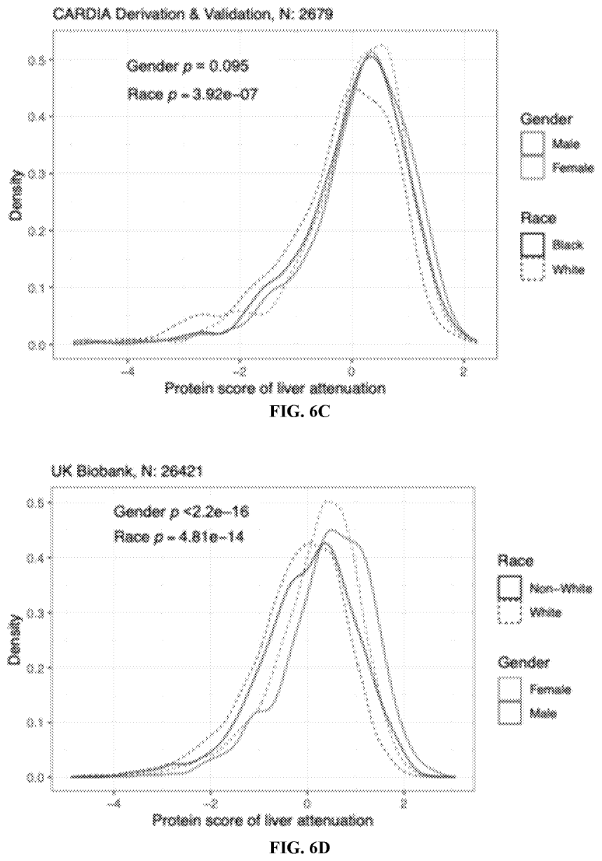

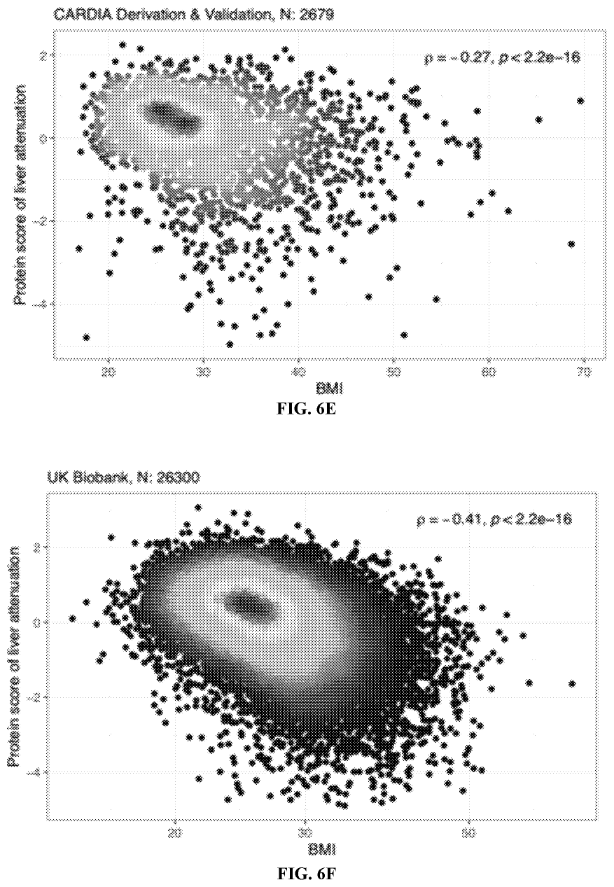

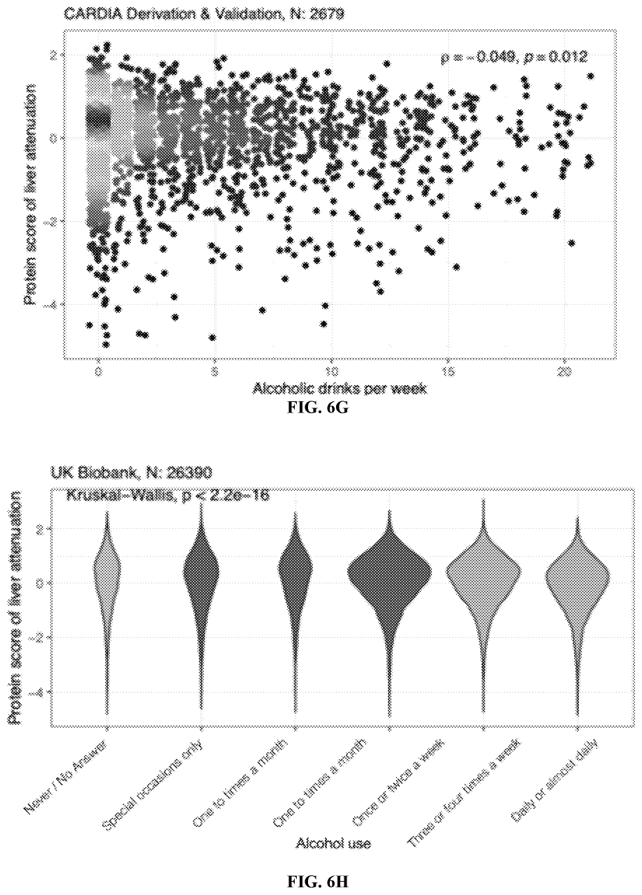

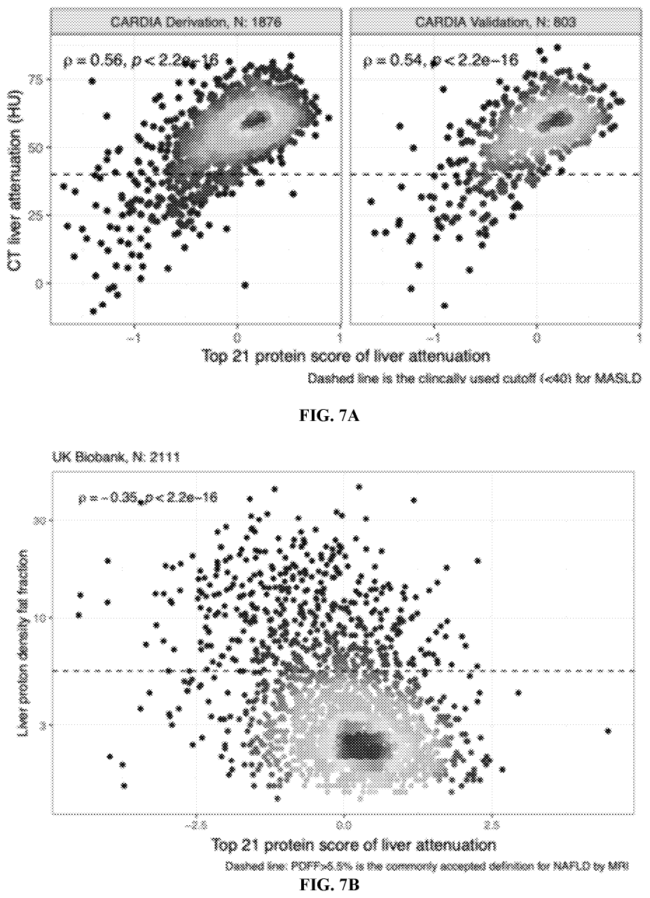

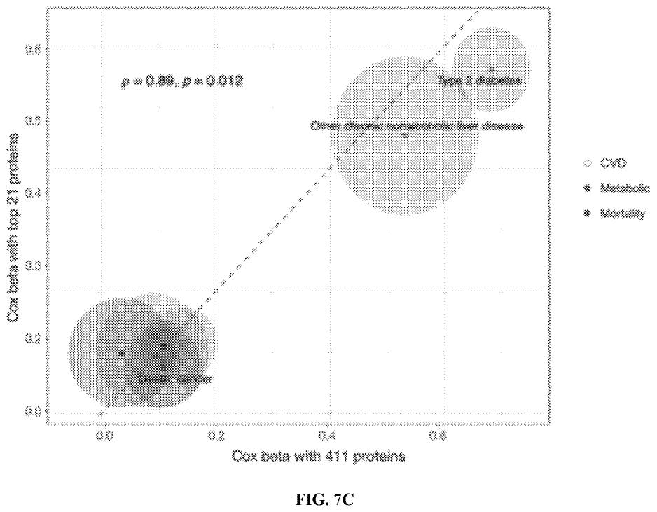

Penalized regression (LASSO) generated a 336-aptamer model for liver attenuation adjusted for age, gender, race, and BMI (hereafter referred to as “MASLD score” for brevity). The MASLD score correlated with liver attenuation in both derivation and validation subsamples within CARDIA (Spearman ρ=0.69 and 0.56, respectively, FIG. 4A), with similar performance when further restricting the validation subsample to participants meeting clinical criteria for MASLD¹¹ (FIG. 5). A modest correlation was observed between BMI and the MASLD score (Spearman ρ=−0.27, P<2.2×10⁻¹⁶), with smaller effects by age, gender, race, and alcohol use (FIG. 6). To develop a clinically translatable MASLD score, a truncated MASLD score was generated using only the top aptamers (ranked by absolute value of beta coefficient; Table 5) which had similar model fit in the CARDIA derivation and validation subsamples (FIG. 7A).

TABLE 5
Biological relevance of top proteins in proteomic score of hepatic steatoses.

| Protein | Coefficient | Prior evidence in hepatic disease |
|---|---|---|
| ANGPT2 | − | Potentiates angiogenesis (pathologic) in MASLD in mice⁷⁸; |
| | | associated with MASLD disease severity in children⁷⁹ |
| ANGPTL3 | − | Higher expression in patients with MASLD; expression related |
| | | to steatosis severity⁸⁰; unclear genetic correlation⁸¹ |
| CES1 | − | Decreased CES1 impacts fatty acid metabolism in the liver, |
| | | potentially implicated in MASLD pathogenesis⁸² |
| CHMP1A | + | No broad evidence |
| CSTZ | − | No broad evidence |
| DNAJB1 | + | No broad evidence |
| FCGR3B | − | Increased levels in hepatic inflammation⁸ |
| GLO1 | + | Decreased expression in mouse model of MASLD, potentially |
| | | via ubiquitin-mediated degradation after exposure to high lipid |
| | | levels⁸³ |
| GSTA1 | − | Dysregulation during MASLD progression⁸⁴ |
| GUSB | − | No broad evidence |
| IGFBP3 | − | Decreased activity by palmitate can increase hepatic |
| | | inflammation⁸⁵; higher circulating levels related to more |
| | | metabolic dysfunction⁸⁶ |
| IL1RN | − | Increased expression in liver related to NASH⁸⁷ |
| MLN | − | No broad evidence |
| NRP1 | − | Expressed in the liver in response to injury⁸⁸ |
| PCSK9 | + | PCKS9 ablation can increase NASH and fibrosis in mice⁸⁹, but |
| | | clinical inhibition may be protective |
| PTPRS | + | No broad evidence |
| RBP5 | − | No broad evidence |
| RELT | + | No broad evidence |
| SEMA4D | + | Deletion can improve liver fibrosis⁹⁰ |
| SMPD1 | − | Inhibition can improve liver function⁹¹ |
| SORD | − | Higher levels indicate liver damage⁹² |

MASLD = metabolic dysfunction associated liver disease; NASH = non-alcoholic steatohepatitis.

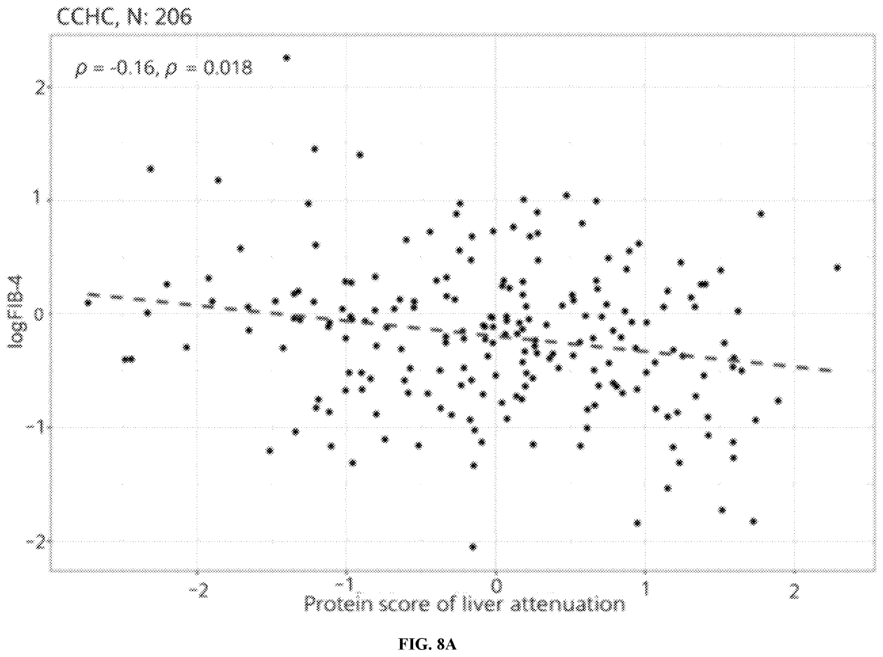

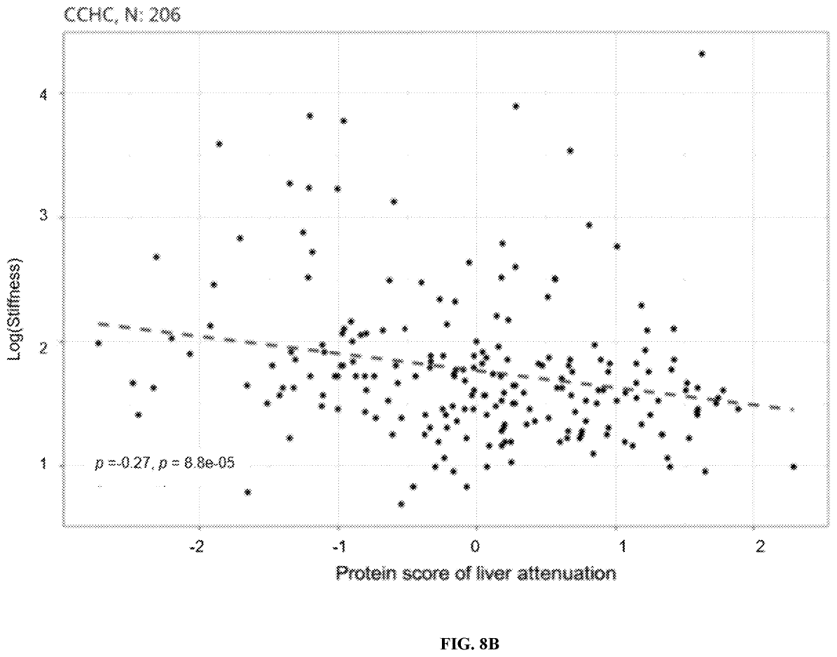

To increase external validity, the relation between the MASLD score and imaging-based indices of hepatic steatosis was measured in >2000 participants from two different studies with distinct approaches to liver fat quantification: ultrasound (controlled attenuation parameter, CCHC) or MRI (proton-derived MRI fat fraction [PDFF], UK Biobank). Of note, CT liver attenuation (the measure in CARDIA) has an opposite directionality relative to ultrasound or MRI (higher attenuation~lower steatosis~lower MRI or ultrasound measure). Largely consistent results were observed in 2111 UK Biobank participants with MRI, including (1) a similar relation between the MASLD score (recalibrated as described in the methods included in the Examples, below) and PDFF (Spearman ρ=−0.5, P<2.2×10⁻¹⁶; FIG. 4B, FIG. 7B). In addition, similar relations were observed between the MASLD score and age, gender, race, BMI, or alcohol use in UK Biobank (FIG. 6). In CCHC (a higher metabolic risk population with far more prevalent MASLD (Table 4)), correlations between MASLD score and ultrasound-based steatosis similar to other cohorts were also observed (Spearman ρ=−0.53, P<2.2×10⁻¹⁶; FIG. 4C). Correlation between the MASLD score and ultrasound-based fibrosis was much weaker than observed with steatosis in CCHC (FIG. 8A-8B).

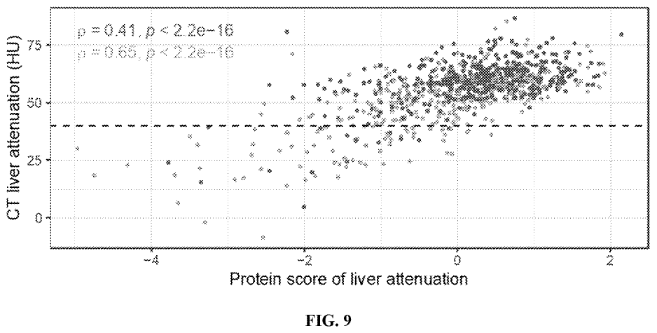

Recognizing the importance of adiposity on the risk of MASLD, whether the relationship between the MASLD score and hepatic steatosis was modified by overweight/obesity status was interrogated. In the CARDIA validation subsample (N=803), a stronger correlation was observed among participants with obesity (BMI≥30 kg/m²; Spearman ρ=0.65) than participants with normal/overweight BMI (<30 kg/m²; Spearman ρ=0.41; FIG. 9). In a linear model for the outcome of hepatic steatosis (which was CT assessed liver attenuation in CARDIA), a statistically significant interaction was observed between the MASLD score and BMI wherein the magnitude of the relationship between the MASLD score and CT liver attenuation increases with greater BMI (interaction β=7.5, P=1.4×10⁻⁸). In conjunction with the observed relationship in CCHC (a cohort with elevated metabolic risk), these findings support the relevance of the MASLD score in high metabolic risk populations.

### Example 4: Relation of MASLD Score and Multi-System Clinical Outcomes

Next, it was examined whether this MASLD score was associated with clinical outcomes, focusing efforts on outcomes known to be associated with hepatic steatosis, including cardiovascular disease, diabetes, and an electronic health record surrogate of MASLD. In 26421 participants in UK Biobank (median follow-up for mortality 13.7 years, 25^{th}-75^{th} percentile 13.0-14.5 years), a broad relation of the MASLD score with MASLD and associated metabolic outcomes was found (FIG. 4D). In addition to all-cause and cause-specific mortality (specifically cancer), very strong relations were observed between the MASLD score (lower score~more steatosis) and chronic non-alcoholic liver diseases (an electronic health record surrogate of MASLD; adjusted standardized hazard ratio [HR]0.59, 95% confidence interval [CI]0.52-0.67; P=7.0×10⁻¹⁶) and diabetes (adjusted standardized HR 0.51, 95% CI 0.47-0.54; P=1.7×10⁻⁸⁶; adjustments in methods described in the Examples below). These associations were robust to additional adjustment for AST, ALT, and hemoglobin Ale and were retained down to a 21-protein Olink panel, the current multiplexing threshold for Olink technology (FIG. 7C). Competing risk models (Fine-Gray subdistribution hazard model) provided similar estimates as cause-specific models for the outcomes of cardiovascular- and cancer-related mortality (cardiovascular mortality HR 0.96, 95% CI 0.88-1.06; cancer mortality HR 0.90, 95% CI 0.84-0.97; Table 6). Addition of the MASLD score significantly improved both discrimination and reclassification metrics above clinical models (including age, gender, race, BMI, systolic blood pressure, diabetes [removed for models of diabetes], Townsend Deprivation Index, smoking, alcohol use, and low-density lipoprotein) for both diabetes and chronic non-alcoholic liver disease (FIG. 41D). Models for incident chronic non-alcoholic liver disease were also robust to further adjustment for AST, ALT and A1c (C-statistic 0.72 vs 0.74, P=0.009).

TABLE 6
Competing risk model results from UK Biobank.

| | | | Hazard | | | |
|---|---|---|---|---|---|---|
| | | | Ratio | | | Number |
| Outcome | Predictor | Adjustments | (95% CI) | P value | N | of events |
| Death; cancer | full_score | unadjusted | 0.79 (0.75- | 7.08e−22 | 26421 | 1155 |
| | | | 0.83) | | | |
| Death; cancer | full_score | age, sex, race, BMI | 0.87 (0.82- | 3.32e−5 | 26300 | 1150 |
| | | | 0.93) | | | |
| Death; cancer | full_score | age, sex, race, | 0.9 (0.84- | 0.00397813 | 23424 | 1006 |
| | | BMI, SBP, | 0.97) | | | |
| | | diabetes, Townsend | | | | |
| | | deprivation index, | | | | |
| | | smoking, alcohol | | | | |
| | | frequency, LDL | | | | |
| Death; cancer | full_score | age, sex, race, | 0.86 (0.79- | 0.00170121 | 22252 | 962 |
| | | BMI, SBP, | 0.95) | | | |
| | | diabetes, Townsend | | | | |
| | | deprivation index, | | | | |
| | | smoking, alcohol | | | | |
| | | frequency, LDL, | | | | |
| | | AST, ALT, HbA1c | | | | |
| Death; cardio- | full_score | unadjusted | 0.7 (0.66- | 5.61e−30 | 26421 | 619 |
| vascular | | | 0.75) | | | |
| Death; cardio- | full_score | age, sex, race, BMI | 0.87 (0.8- | 7.70e−4 | 26300 | 608 |
| vascular | | | 0.94) | | | |
| Death; cardio- | full_score | age, sex, race, | 0.96 (0.88- | 0.41715648 | 23424 | 533 |
| vascular | | BMI, SBP, | 1.06) | | | |
| | | diabetes, Townsend | | | | |
| | | deprivation index, | | | | |
| | | smoking, alcohol | | | | |
| | | frequency, LDL | | | | |
| Death; cardio- | full_score | age, sex, race, | 0.88 (0.79- | 0.03000415 | 22252 | 513 |
| vascular | | BMI, SBP, | 0.99) | | | |
| | | diabetes, Townsend | | | | |
| | | deprivation index, | | | | |
| | | smoking, alcohol | | | | |
| | | frequency, LDL, | | | | |
| | | AST, ALT, HbA1c | | | | |

CI = confidence interval; BMI = body mass index; SBP = systolic blood pressure; LDL = low density lipoprotein; AST = aspartate aminotransferase; ALT = alanine aminotransferase; HbA1c = hemoglobin A1c.

### Example 5: Spatial and Cellular Organization of the Hepatic Steatosis Proteome

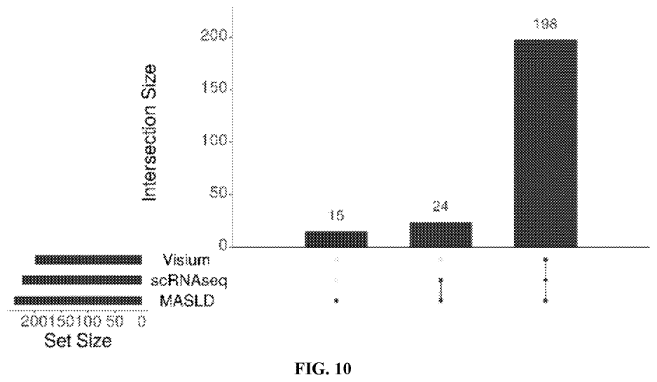

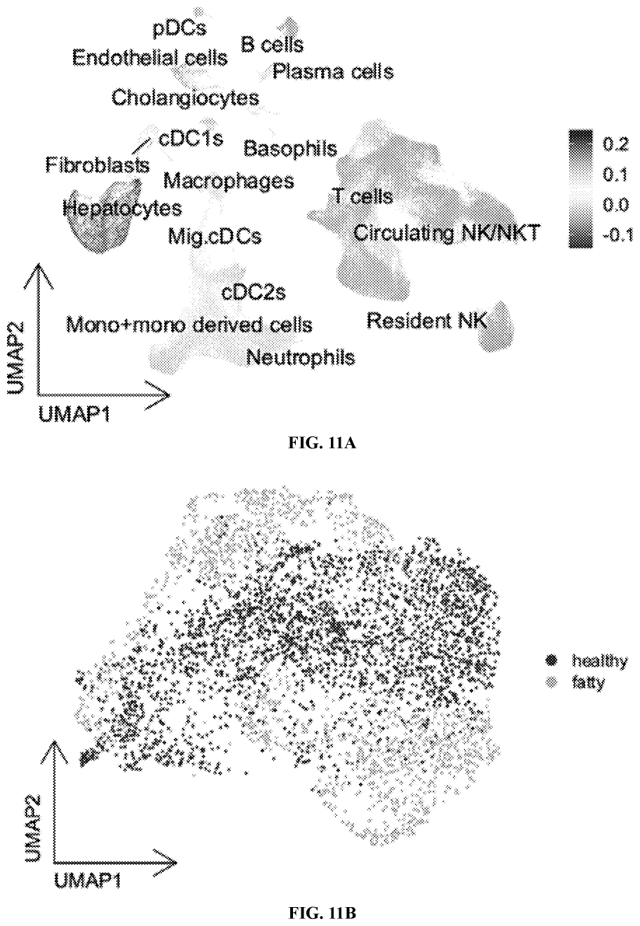

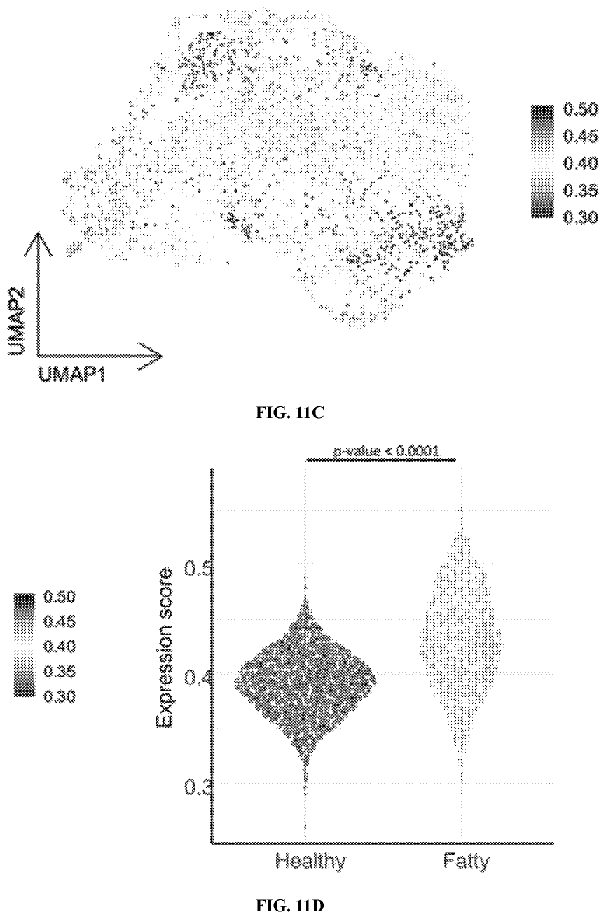

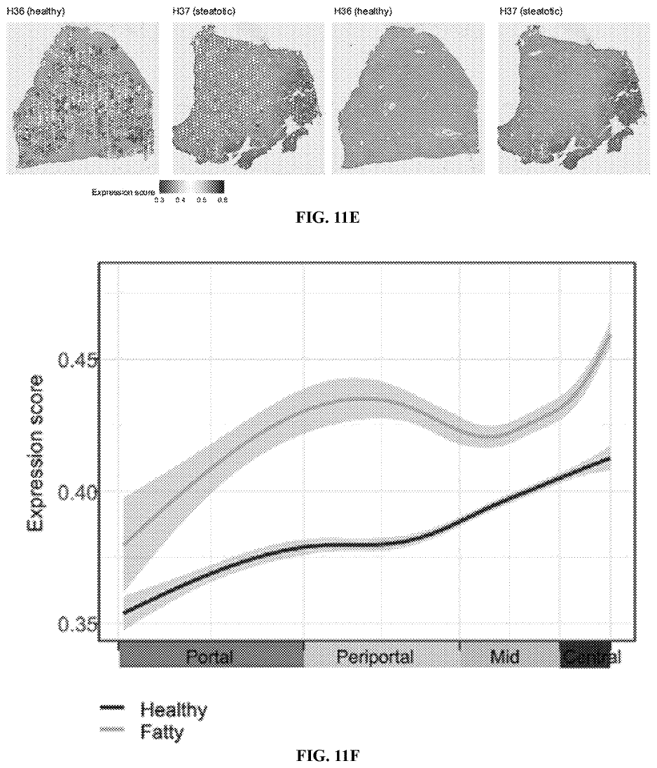

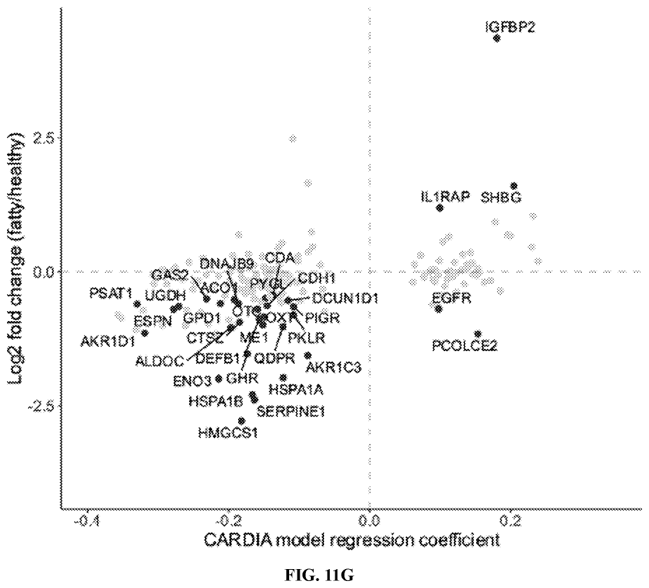

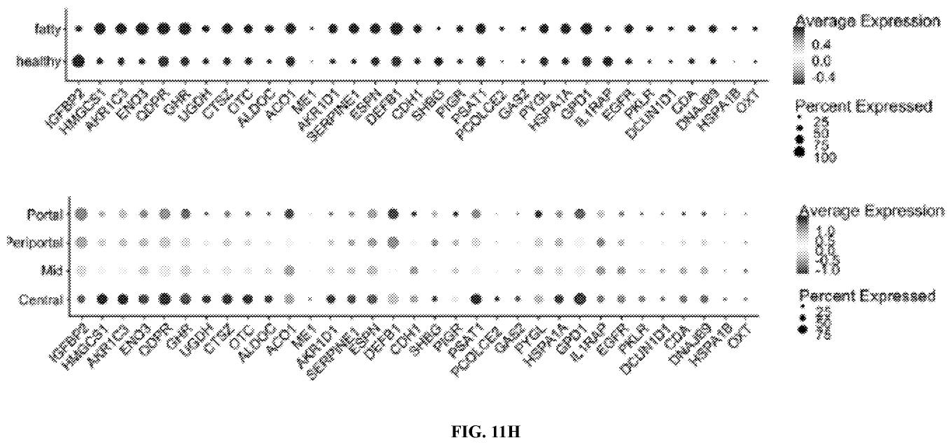

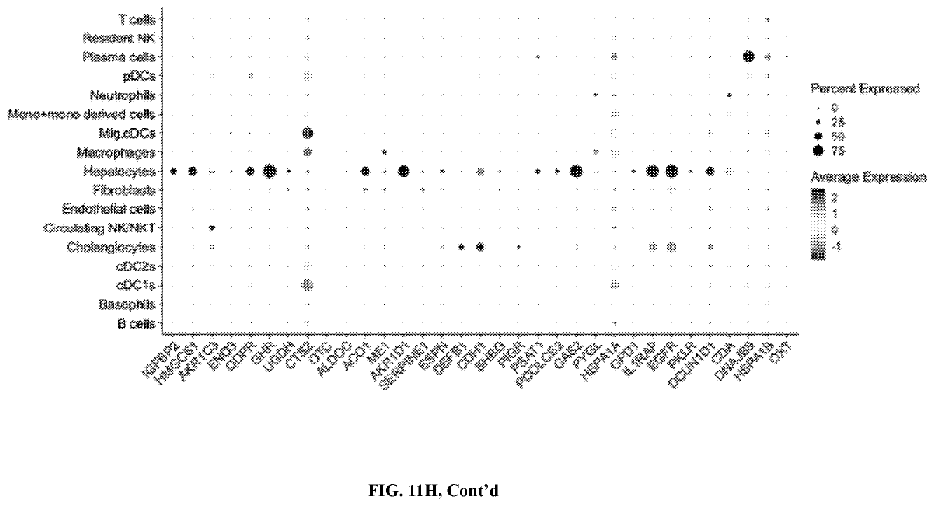

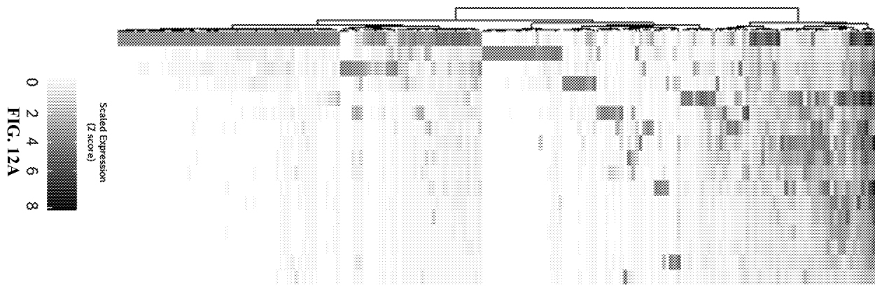

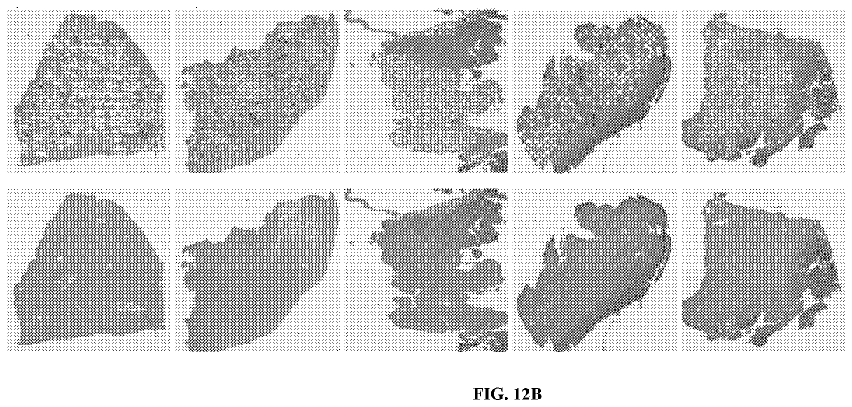

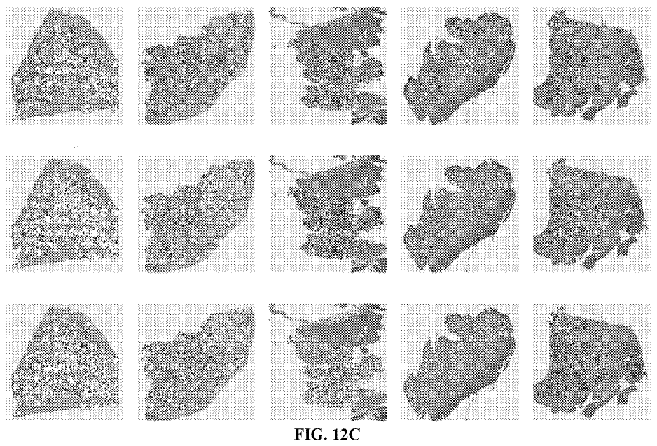

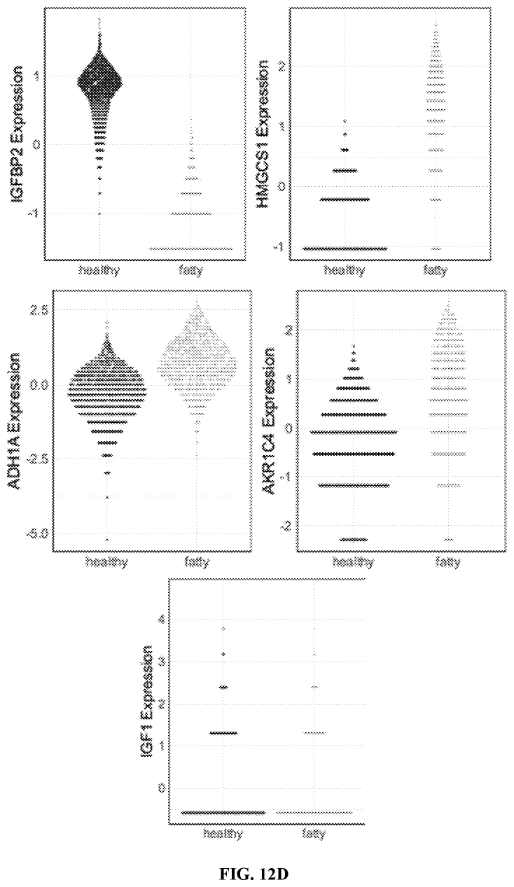

To elucidate the spatial and cellular organization of prioritized targets identified in proteomic analyses, proteins identified from clinical proteomic regressions in CARDIA were mapped to their gene expression in human liver by leveraging published integrated single-cell and single-nuclear RNA sequencing (N=19; single-cell RNA-seq N=14; single-nuclear RNA-seq N=5, hereafter referred to as scRNA-seq) and spatial transcriptomics (N=4) data from the Liver Cell Atlas (www.livercellatlas.org)³⁴. Two samples had both spatial transcriptomics and scRNA-seq data resulting in samples from 21 unique participants (62% women, mean age 59 years, mean BMI 32 kg/m²) used from the Liver Cell Atlas. To facilitate the broad identification of cell types and spatial distribution of targets prioritized by their relations with hepatic steatosis within the human liver, the expression profiles of 198 of the 237 steatosis-associated proteins that were expressed in both the scRNA-seq and spatial transcriptomics datasets were investigated (FIG. 10) using individual gene expression and a composite expression score (FIG. 11). This analysis suggested a predominant cell-specific expression pattern of implicated targets, primarily observed in hepatocytes, with a subset of targets expressed across cell types, including fibroblasts, cholangiocytes, endothelial cells, and immune cells (FIG. 11A and FIG. 12A). Employing a composite expression score, higher gene activity of implicated targets was shown within steatotic tissue and in the mid-central liver zonation. These zones were previously shown to correspond to higher hepatocyte expression signature in this dataset (FIG. 11B-11F, FIG. 12B)³⁴.

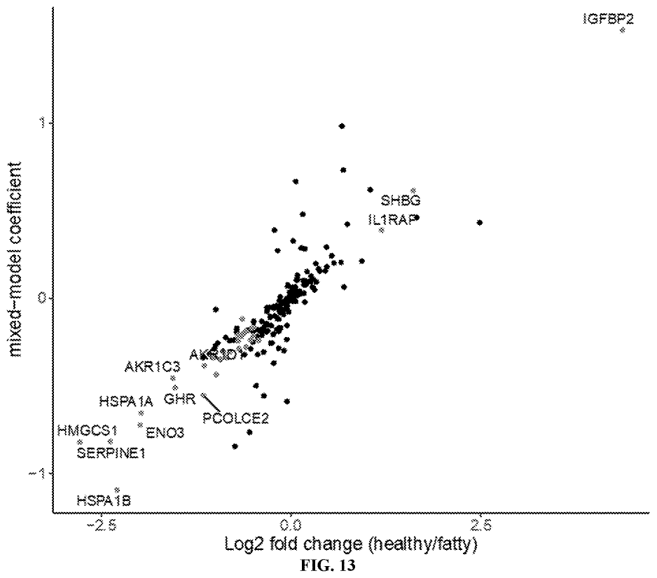

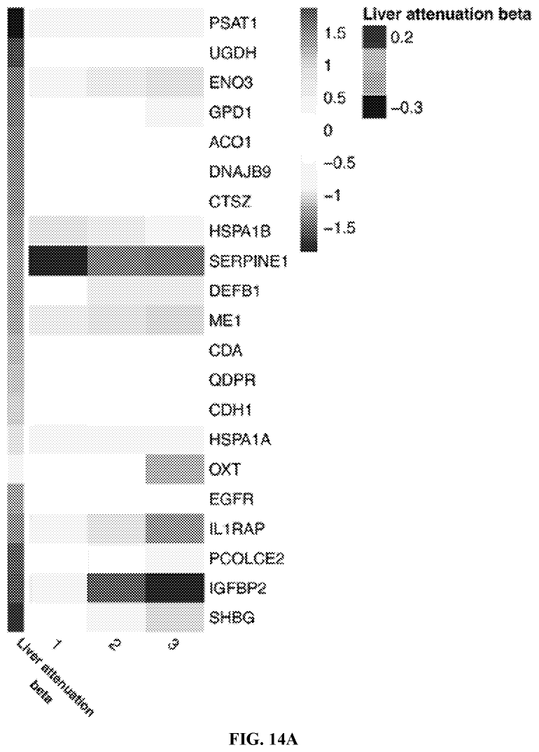

Next, it was tested whether implicated targets identified by proteomics in CARDIA would exhibit differential expression in liver tissue by spatial transcriptomics. Of the 198 steatosis-associated protein-gene overlaps expressed in spatial transcriptomics data, 33 were differentially expressed between healthy and steatotic tissue (minimum expression >10% of spots; FDR adjusted P<0.05; |log₂ fold-change|>0.25; and |minimum % expressed spot difference|>10%; FIG. 11H). Of the 33 genes, 30 were upregulated in steatotic tissue and 3 genes (IGFBP2, IL1RAP and SHBG) were downregulated. These genes exhibited distinct expression patterns in human liver tissue with and without steatosis (FIG. 11H, select targets shown in FIG. 12C-12D. Moreover, biological directional concordance was observed between the difference in RNA expression between fatty (relative to non-fatty) areas in human liver and the clinical effect size of the circulating protein in CARDIA (FIG. 11G; FIG. 13; e.g., greater positive CARDIA regression coefficient means higher protein~lower steatosis~higher tissue RNA expression in healthy vs. fatty liver areas). One notable example was IGFBP2—dynamic during metabolic intervention³⁵ and regeneration³⁶ with liver-enriched expression³⁷—which was increased in healthy versus steatotic cell populations (at a transcriptional level, FIG. 12D) and exhibited higher levels associated with less steatosis in CARDIA (at a population proteomic level), consistent with smaller reports³⁸. Most observed population association-tissue concordance consisted of metabolic genes up-regulated in steatotic liver that displayed greater circulating protein abundance in individuals with greater steatosis (FIG. 11G-11H). Many of these targets had established mechanistic relevance in model systems of MASLD and its progression (e.g., ENO3 and ferroptosis³⁹; UGDH and fibrosis/redox status²³; CTSZ and epithelial-mesenchymal transition⁴⁰; CDH1 and lipogenesis⁴¹; CDH1 and PPAR/PGC1α signaling⁴²; among others).

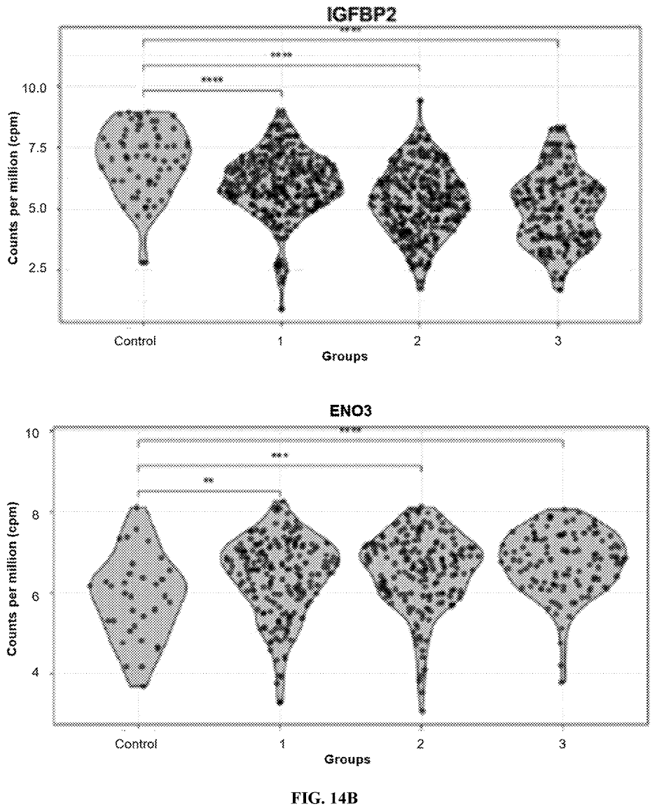

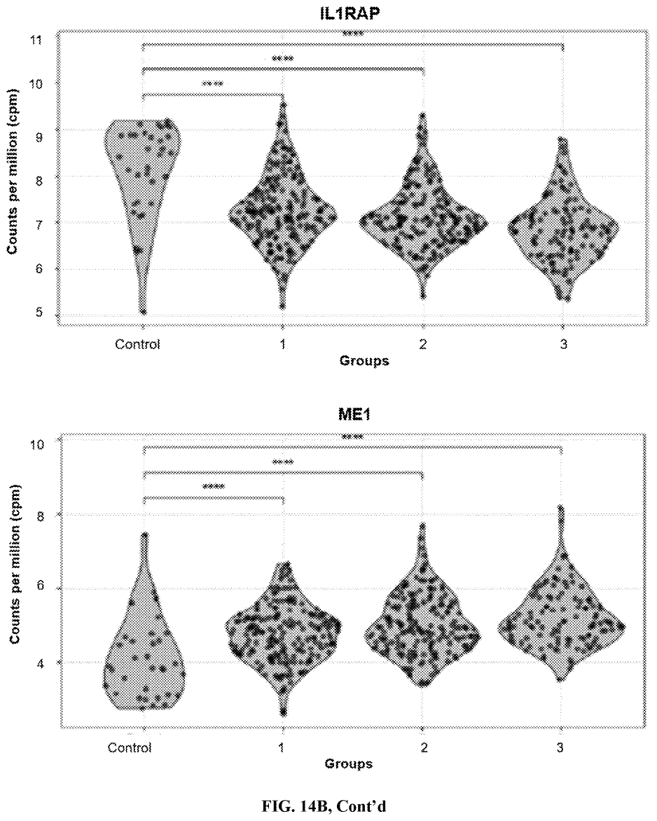

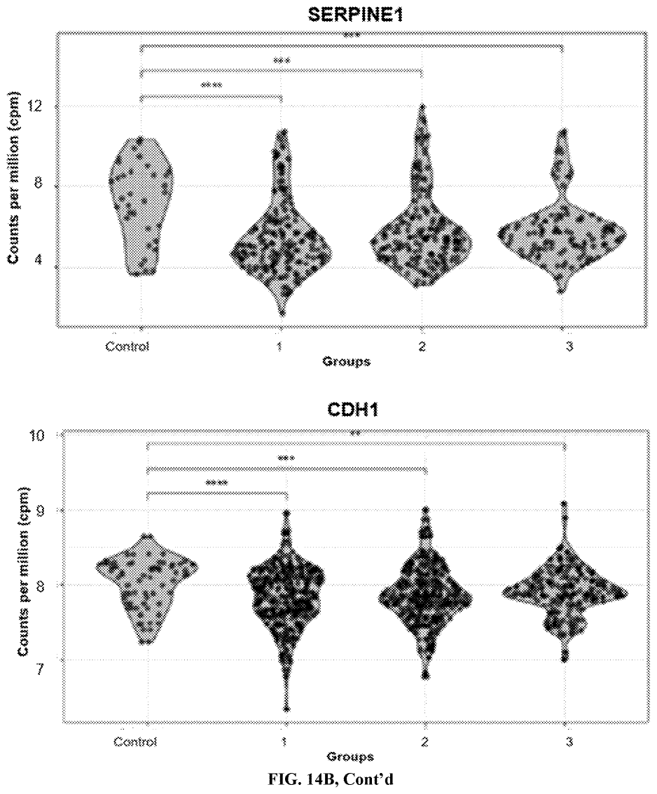

Next, these 33 differentially expressed genes were matched to bulk transcriptomic data across NASH-CRN defined stages of hepatic steatosis to investigate potential dynamicity across individuals with increasing severity of histopathologic phenotype (FIG. 14A-14B). 499 SteatoSITE participants were compared with MASLD and biopsy samples (47% women, median age 53 years, median BMI 31 kg/m², 31% diabetes; Table 7) to 34 control samples. Of the 33 genes passed forward for assessment in bulk transcriptomics, 12 were not significantly expressed in any of the stages of steatosis (by FDR adjusted P<0.05) and were not included in visualization (FIG. 14A-14B).

TABLE 7
Characteristics of SteatoSITE cohort.

| Variable | Value | |
|---|---|---|
| Age, median (IQR) | | |
| Years | 53 | (45, 63) |

Gender, number (%)

| | | |
|---|---|---|
| Male | 264 | (52.9) |
| Female | 235 | (47.1) |

Ethnicity, number (%)

| | | |
|---|---|---|
| African | 1 | (0.2) |
| Asian | 13 | (2.6) |
| Unknown | 130 | (26.1) |
| White | 355 | (71.1) |

Body mass index, median (IQR)

| | | |
|---|---|---|
| kg/m² | 31 | (27.9, 35) |

| | |
|---|---|
| Unknown BMI, number | 346 |

Diabetes, number (%)

| | | |
|---|---|---|
| Yes | 156 | (31.3) |
| No | 343 | (68.7) |

Transient elastography, median (IQR)

| | | |
|---|---|---|
| kPa | 12.3 | (8.3, 20) |

NASH-CRN fibrosis stage, number (%)

| | | |
|---|---|---|
| 0 | 150 | (30.1) |
| 1 | 111 | (22.2) |
| 2 | 77 | (15.4) |
| 3 | 87 | (17.4) |
| 4 | 74 | (14.8) |

NAFLD Activity Score Steatosis, number (%)

| | | |
|---|---|---|
| 0 | 17 | (3.4) |
| 1 | 190 | (38.1) |
| 2 | 183 | (36.7) |
| 3 | 109 | (21.8) |

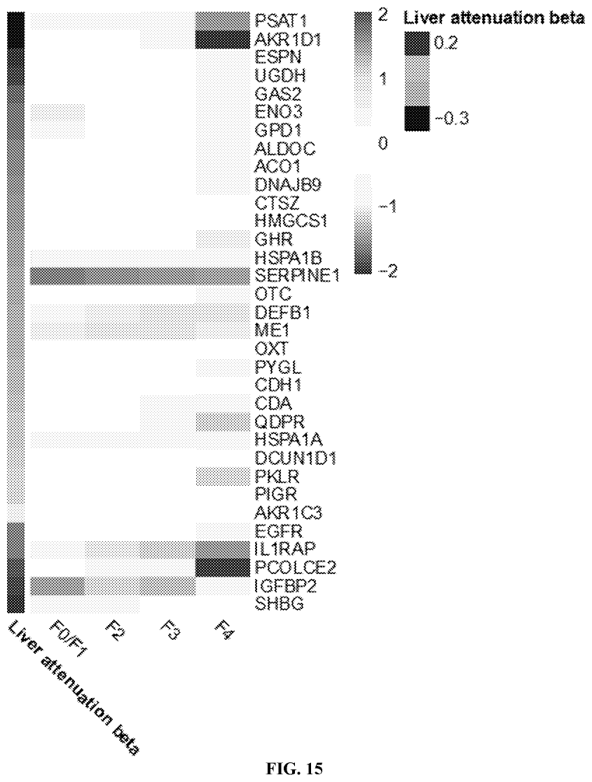

Several genes with high effect size differences by steatosis grade were observed, concordant with circulating proteomic and spatial relation (e.g., IGFBP2, IL1RAP, SHBG, ENO3, DEGB1, ME1), some of which were also concordant across fibrotic stages (FIG. 15). Across the genes prioritized by proteomic and spatial studies, two major classes of discordant findings were observed: (1) genes directionality consistent with the proteomic-spatial but not bulk transcriptional results (e.g., SERPINE1/PAI-1⁴³⁻⁴⁵, HSPA1B⁴⁶); (2) genes consistent with the bulk but not proteomic-spatial directionality (e.g., PSAT1⁴⁷, UDGH²³, ACO1⁴⁸). Several factors-technical (sequencing methodologies, bulk versus single cell, limited sample size in this previously published scRNAseq dataset³⁴, participant-level, and biological (steatosis as one component of the MASLD phenotype, in addition to inflammation, ballooning, fibrosis)—may account for these differences. Nevertheless, these findings collectively highlight the potential for proteo-transcriptional target mapping for human MASLD amidst context-dependent heterogeneity and complexity of integrating multiple approaches.

### Example 6: Humanized Liver-On-a-Chip of Human Steatosis

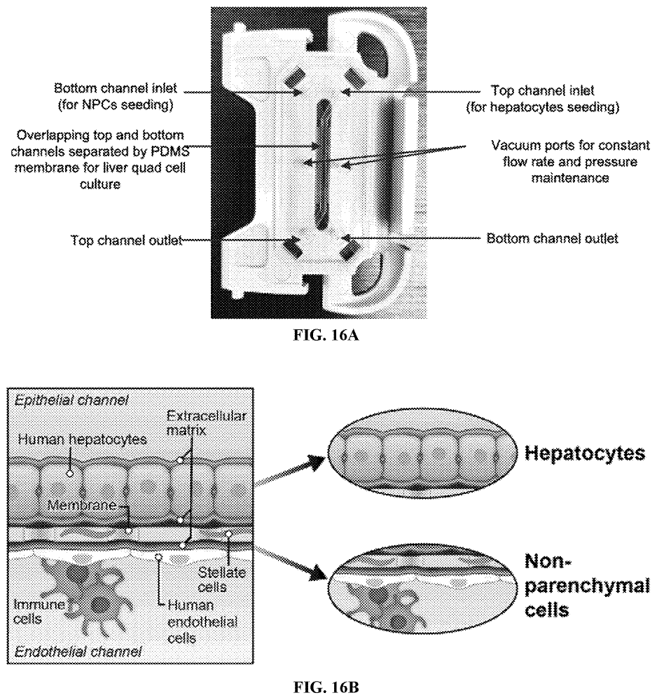

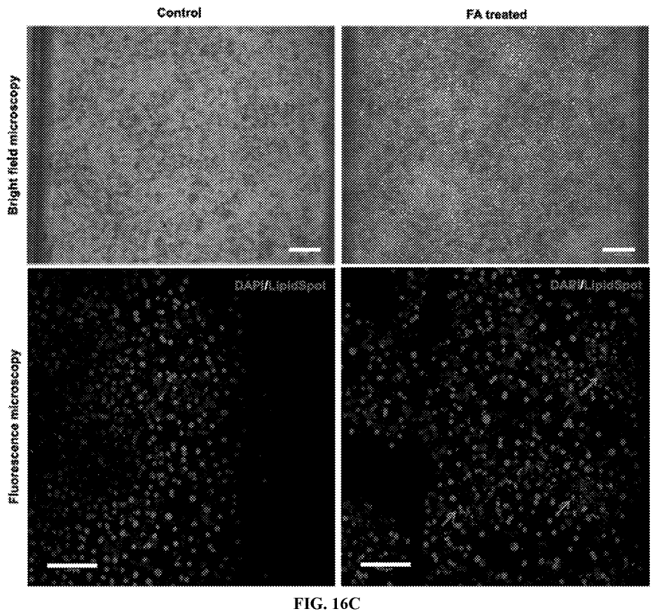

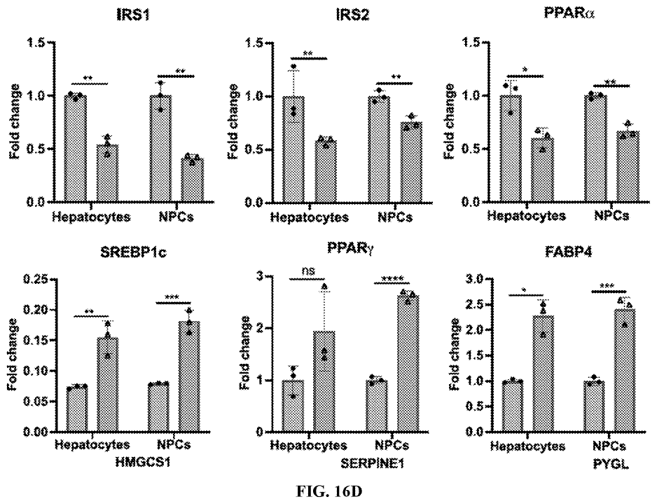

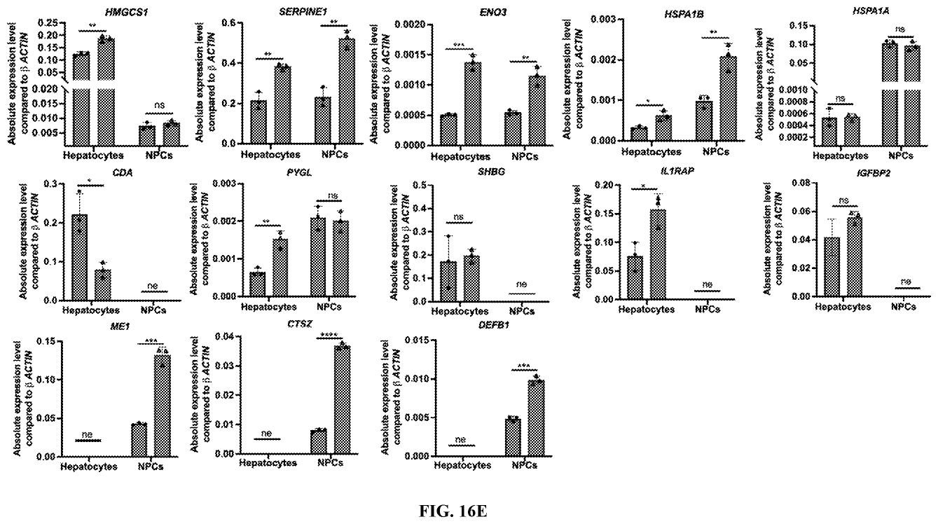

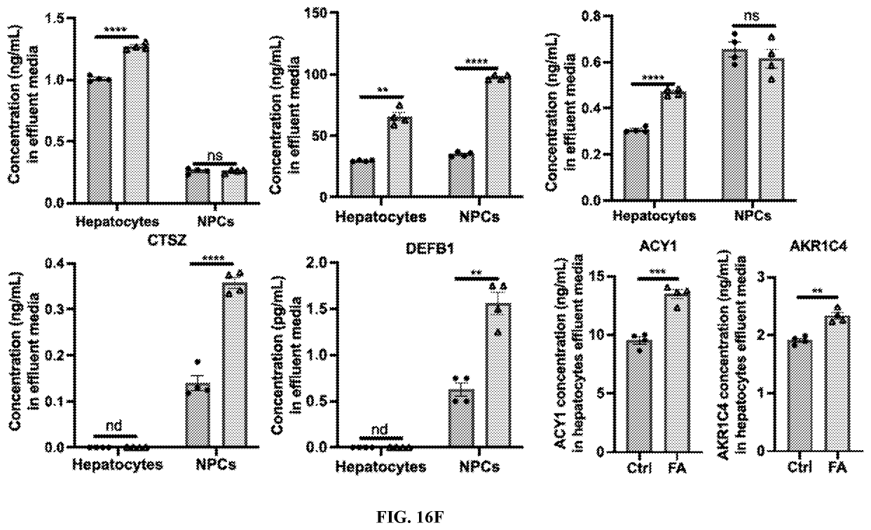

To demonstrate the direct association of transcriptional changes in steatosis-implicated genes in different liver cells with the MASLD proteome, a humanized liver-on-a-chip (LOC) model of steatosis was developed. The experimental design for the LOC studies is shown in FIG. 16A-16B. Microscopy, immunofluorescence, and gene expression studies after administration of fatty acids (oleic and palmitic) known to promote a steatosis phenotype⁴⁹ were consistent with morphologic and transcriptomic induction of a MASLD phenotype (decreased IRS1^{50,51}, IRS2⁵¹, PPARα^{52,53}; increased SREBP1c⁵⁴, PPARg⁵⁵, FABP4⁵⁶; FIG. 16C-16D). Given limited cDNA yield from individual LOC experiments, 13 of 33 targets identified across proteomic and spatial transcriptomic studies were prioritized for assessment on the LOC in two ways: (1) 5 targets that were the most upregulated in steatosis (HMGCS1, SERPINE1, HSPA1B, ENO3, HSPA1A), plus all 3 targets which were downregulated in steatosis (IL1RAP, SHBG, IGFBP2), and the 2 targets that were the least upregulated in steatosis (CDA, PYGL) by spatial transcriptomics; (2) three additional targets differentially expressed in spatial human liver studies but with prominent expression in non-parenchymal (non-hepatocyte; NPCs) cells (ME1, CTSZ, DEFB1). In addition, two hepatocyte-specific targets identified from the circulating proteomic relations to hepatic steatosis in CARDIA (ACY1, AKR1C4) that were strongly correlated with steatosis and had a high confidence as secretory proteins were assayed, although these did not meet the pre-specified cut-offs for prioritization in the transcriptional studies.

Broadly directionally consistent results were observed between circulating proteomic, tissue transcriptional, and LOC experiments, with increased expression of genes implicated in hepatic lipid metabolism, stress, and non-canonical pathways (e.g., ferroptosis) in hepatocytes and/or NPCs (PYGL, HMGCS1, SERPINE1, ENO3, HSPA1B; FIG. 16E). Furthermore, while ME1, CSTZ, and DEFB1 were not expressed in LOC hepatocytes (consistent with transcriptomic studies), the expression of these genes was increased in NPCs in the LOC (FIG. 16E). Together with bulk data for DEFB1 and ME1 in bulk transcriptional data (FIG. 14A-14B), these results suggest increased expression of these genes may be predominantly driven by cells of non-hepatocyte origin. Of note, several genes did not exhibit the expected directionality from proteomic, spatial, or bulk studies (non-significant: IGFBP2, SHBG; opposite directionality, IL1RAP, CDA; FIG. 16E), potentially owing to biological heterogeneity between model systems. In the case of ACY1 and AKR1C4, the LOC suggested a higher sensitivity for detection of gene expression changes with steatosis induction than were detected in human tissue studies. However, several genes did not exhibit the expected directionality from proteomic, spatial, or bulk studies (non-significant changes: IGFBP2, SHBG; opposite directionality, IL1RAP, CDA; FIG. 16E), potentially owing to biological heterogeneity between model systems and time points corresponding to different disease stages.

To assess whether steatosis induction on the LOC led to an increase in expression of these targets at a protein level (connecting with the population studies), selected targets were measured in the effluent of hepatocytes and NPCs uniquely accessible on the LOC. Seven of 15 genes differentially expressed in hepatocytes or NPCs at the mRNA level on LOC experiments that had (1) cellular protein targets with high confidence for extracellular secretion, (2) commercially available ELISA kits for assessment on the effluents of the LOC experiments (HMGCS1, SERPINE1, PYGL, CTSZ, DEFB1, ACY1, and AKR1C4; FIG. 16, Tables 8 and 9) were prioritized. Concordant expression was noted at the protein level of HMGCS1, SERPINE1, CTSZ, DEFB1, PYGL, ACY1, and AKR1C4 in cellular effluents of LOC (FIG. 16F), indicating cell-type specific protein secretion that matched transcriptional changes on the LOC and proteomic directionality at the population level. Interestingly, secreted proteins encoded by genes specific to non-hepatocytes in the transcriptomic data (and validated at the mRNA expression level in THE LOC data) were only detected in the effluents from the non-hepatocyte cells (CTSZ, DEFB1). Conversely, hepatocyte-specific proteins were detected only in the hepatocyte channel (ACY1, AKR1C4).

TABLE 8
Predicted secretory proteins.

| | | Direction (at | Extracellular |
|---|---|---|---|
| Sl. | Target | transcript level | expression confidence |
| No. | Name | in LOC) | level (Genecards) |
| 1. | SERPINE1 | Increase | 5 |
| 2. | CTSZ | Increase | 5 |
| 3. | DEFB1 | Increase | 5 |
| 4. | PYGL | Increase | 4 |
| 5. | HMGCS1 | Increase | 2 |
| 6. | ACY1 | Increase | 5 |
| 7. | AKR1C4 | Increase | 4 |

TABLE 9
ELISA OD (at 450 nm) values of liver-on-a-chip effluent media.
SERPINE1

| | | Inlet | Inlet | Control 1 | Control 2 | FA 1 | FA 2 | |
|---|---|---|---|---|---|---|---|---|
| | | Media | Media | (1:50 | (1:50 | (1:50 | (1:50 | Channel |
| Standard 1 | Standard 2 | Control | FA | dilution) | dilution) | dilution) | dilution) | name |
| 3.309 | 3.255 | 0.155 | 0.151 | 1.112 | 1.126 | 2.339 | 2.046 | Hepatocyte |
| 1.717 | 1.848 | 0.157 | 0.158 | 1.162 | 1.135 | 2.14 | 2.006 | channel |
| 0.993 | 0.916 | 0.163 | 0.168 | 1.163 | 1.168 | 2.265 | 2.28 | |
| 0.527 | 0.554 | 0.134 | 0.139 | 1.097 | 1.099 | 2.564 | 2.563 | |
| 0.337 | 0.317 | 0.145 | 0.142 | 1.343 | 1.317 | 3.37 | 3.334 | NPC channel |
| 0.199 | 0.192 | 0.125 | 0.17 | 1.365 | 1.372 | 3.74 | 2.98 | |
| 0.142 | 0.151 | 0.152 | 0.376 | 1.373 | 1.366 | 3.39 | 3.332 | |
| 0.094 | 0.085 | 0.135 | 0.408 | 1.345 | 1.378 | 3.346 | 3.38 | |

HMGCS1

| | | Inlet | Inlet | | | | | |
|---|---|---|---|---|---|---|---|---|
| | | Media | Media | | | | | Channel |
| Standard 1 | Standard 2 | Control | FA | Control 1 | Control 2 | FA 1 | FA 2 | Name |
| 3.956 | 3.956 | 0.1 | 0.1 | 0.631 | 0.646 | 0.747 | 0.743 | Hepatocyte |
| 2.255 | 2.261 | 0.112 | 0.113 | 0.638 | 0.642 | 0.741 | 0.734 | channel |
| 1.269 | 1.269 | 0.116 | 0.116 | 0.623 | 0.646 | 0.742 | 0.748 | |
| 0.785 | 0.785 | 0.113 | 0.115 | 0.638 | 0.639 | 0.731 | 0.739 | |
| 0.335 | 0.335 | 0.116 | 0.116 | 0.357 | 0.338 | 0.343 | 0.352 | NPC channel |
| 0.166 | 0.166 | 0.11 | 0.112 | 0.329 | 0.363 | 0.343 | 0.347 | |
| 0.113 | 0.113 | 0.112 | 0.112 | 0.399 | 0.303 | 0.336 | 0.334 | |
| 0 | 0 | 0.105 | 0.105 | 0.331 | 0.317 | 0.336 | 0.343 | |

PYGL

| | | Inlet | Inlet | | | | | |
|---|---|---|---|---|---|---|---|---|
| | | Media | Media | | | | | |
| Standard 1 | Standard 2 | Control | FA | Control 1 | Control 2 | FA 1 | FA 2 | |
| 3.96 | 3.916 | 0.179 | 0.18 | 0.392 | 0.378 | 0.419 | 0.413 | Hepatocyte |
| 2.467 | 2.496 | 0.174 | 0.177 | 0.379 | 0.385 | 0.417 | 0.411 | channel |
| 1.32 | 1.312 | 0.171 | 0.173 | 0.39 | 0.367 | 0.411 | 0.419 | |
| 0.792 | 0.783 | 0.174 | 0.171 | 0.382 | 0.38 | 0.414 | 0.411 | |
| 0.362 | 0.367 | 0.173 | 0.173 | 0.409 | 0.463 | 0.451 | 0.419 | NPC channel |
| 0.169 | 0.16 | 0.171 | 0.171 | 0.454 | 0.453 | 0.435 | 0.408 | |
| 0.137 | 0.13 | 0.178 | 0.174 | 0.462 | 0.465 | 0.451 | 0.442 | |
| 0.03 | 0.001 | 0.177 | 0.177 | 0.439 | 0.453 | 0.456 | 0.472 | |

CTSZ

| | | Inlet | Inlet | | | | | |
|---|---|---|---|---|---|---|---|---|
| | | Media | Media | | | | | |
| Standard 1 | Standard 2 | Control | FA | Control 1 | Control 2 | FA 1 | FA 2 | |
| 3.765 | 3.916 | 0.179 | 0.18 | 0.192 | 0.178 | 0.199 | 0.193 | Hepatocyte |
| 2.216 | 2.206 | 0.185 | 0.187 | 0.178 | 0.199 | 0.197 | 0.191 | channel |
| 1.302 | 1.312 | 0.174 | 0.173 | 0.17 | 0.187 | 0.191 | 0.199 | |
| 0.792 | 0.783 | 0.172 | 0.171 | 0.182 | 0.173 | 0.194 | 0.198 | |
| 0.378 | 0.361 | 0.173 | 0.173 | 0.379 | 0.383 | 0.451 | 0.459 | NPC channel |
| 0.162 | 0.162 | 0.175 | 0.177 | 0.374 | 0.353 | 0.435 | 0.448 | |
| 0.127 | 0.134 | 0.177 | 0.179 | 0.362 | 0.365 | 0.451 | 0.442 | |
| 0.008 | 0.03 | 0.174 | 0.174 | 0.369 | 0.343 | 0.456 | 0.452 | |

DEFB1

| | | Inlet | Inlet | | | | | |
|---|---|---|---|---|---|---|---|---|
| | | Media | Media | | | | | |
| Standard 1 | Standard 2 | Control | FA | Control 1 | Control 2 | FA 1 | FA 2 | |
| 3.982 | 3.95 | 0.132 | 0.133 | 0.249 | 0.258 | 0.249 | 0.243 | Hepatocyte |
| 2.422 | 2.426 | 0.13 | 0.132 | 0.259 | 0.245 | 0.257 | 0.241 | channel |
| 1.305 | 1.236 | 0.125 | 0.123 | 0.239 | 0.246 | 0.241 | 0.249 | |
| 0.77 | 0.772 | 0.118 | 0.116 | 0.232 | 0.245 | 0.234 | 0.241 | |
| 0.366 | 0.362 | 0.131 | 0.132 | 0.256 | 0.259 | 0.261 | 0.257 | NPC channel |
| 0.166 | 0.161 | 0.126 | 0.128 | 0.254 | 0.253 | 0.25 | 0.261 | |
| 0.116 | 0.112 | 0.128 | 0.128 | 0.257 | 0.249 | 0.254 | 0.255 | |
| 0.02 | 0.007 | 0.13 | 0.131 | 0.259 | 0.253 | 0.257 | 0.26 | |

AKR1C4 (Hepatocyte channel only)

| | | Blank for | | | | | |
|---|---|---|---|---|---|---|---|
| Standard 1 | Standard 2 | Control | Blank for FA | Control1 | Control 2 | FA 1 | FA 2 |
| 3.79 | 3.703 | 0.054 | 0.03 | 0.705 | 0.751 | 0.998 | 1.087 |
| 2.003 | 2.007 | 0.034 | 0.033 | 0.719 | 0.748 | 0.947 | 0.978 |
| 1.03 | 1.15 | 0.034 | 0.041 | 0.767 | 0.736 | 1.042 | 0.966 |
| 0.745 | 0.746 | 0.031 | 0.033 | 0.767 | 0.766 | 0.942 | 0.966 |
| 0.403 | 0.404 | | | | | | |
| 0.165 | 0.159 | | | | | | |
| 0.11 | 0.105 | | | | | | |
| 0.033 | 0.048 | | | | | | |

ACY1(Hepatocyte channel only)

| | | Blank for | | | | | |
|---|---|---|---|---|---|---|---|
| Standard 1 | Standard 2 | Control | Blank for FA | Control 1 | Control 2 | FA 1 | FA 2 |
| 3.179 | 3.503 | 0.254 | 0.203 | 0.805 | 0.871 | 0.808 | 0.987 |
| 2.203 | 2.28 | 0.234 | 0.233 | 0.839 | 0.872 | 0.967 | 0.968 |
| 1.437 | 1.45 | 0.234 | 0.241 | 0.867 | 0.85 | 1.001 | 0.956 |
| 0.995 | 0.986 | 0.231 | 0.233 | 0.877 | 0.806 | 0.987 | 0.971 |
| 0.643 | 0.654 | | | | | | |
| 0.465 | 0.487 | | | | | | |
| 0.331 | 0.305 | | | | | | |
| 0.238 | 0.248 | | | | | | |

### Example 7: Experimental Model and Study Participants

Study samples. The study involved multiple cohorts: (1) the Coronary Artery Risk Development in Young Adults study (CARDIA, N=2679; proteomic discovery and validation of proteins related to hepatic steatosis; characteristics in Table 2, study design in reference); (2) the UK Biobank study (N=26421; second validation of MASLD score; assessment of clinical prognostic value against incident MASLD-related diseases; characteristics in Table 3); (3) Cameron County Hispanic Cohort (CCHC; N=206 with ultrasound-based measures of liver structure and circulating proteomics; characteristics in Table 4); (4) published bulk RNA sequencing study (SteatoSITE, N=499 liver biopsies across stages of MASLD; characteristics in Table 7)⁹; (5) integrated single-cell and single-nuclear RNA sequencing obtained from the liver cell atlas (www.livercellatlas.org, N=19; single-cell RNA-seq, N=14; single-nucleus RNA-seq, N=5) and spatial transcriptomic study in human liver (5 samples from 4 individuals)³⁴.

CARDIA: The Coronary Artery Risk Development in Young Adults (CARDIA) study started recruitment in 1985-1986 across 4 cities in the U.S. (Birmingham, AL; Chicago, IL; Minneapolis, MN; and Oakland, CA) to study coronary risk factor development longitudinally beginning in young adulthood⁷⁸⁻⁸¹. The study used data from the Year 25 exam where 2977 participants had proteomics quantified. 275 participants with other potential causes for hepatics steatosis (>14 alcoholic drinks/week, hepatitis C, cirrhosis, HIV, and use of amiodarone, valproic acid, methotrexate, tamoxifen, or diltiazem)¹⁰ were excluded. 11 participants missing hepatic steatosis measurements were excluded, and 12 participants were excluded for missing data on BMI or drinks/week. CARDIA participants were randomly split into derivation (70%) and validation (30%) samples, balanced by computed tomography-based measurement of hepatic steatosis.

UK Biobank: The UK Biobank is a population-based study of >500000 participants who were aged 40-69 when recruited between 2006-2010 across the United Kingdom. Proteomics data from the initial assessment (instance 0) using the Olink Explore panel is available on &54000 UK Biobank participants⁸². 26429 participants with complete data for the proteins used to calculate the MASLD score were included, of which 8 participants were excluded from analyses for having a MASLD score >5 SDs away from the mean. A subset of 2111 had hepatic steatosis quantified by MRI at the imaging visit (2014 and later; instance 2).

Cameron County Hispanic Cohort (CCHC): The CCHC is a community-based prospective observational cohort study of 5122 individuals (age 8-90) from a low-income Hispanic/Latino population at the Texas/Mexico border. The study design has been previously described⁸³. 206 participants were included who had abdominal ultrasound to measure controlled attenuation parameter (CAP), a quantitative measure of hepatic steatosis⁸⁴, and simultaneous circulating proteomics.

Ethics: All study participants provided written and informed consent, and all study protocols were approved by the Institutional Review Boards of the respective studies. Approval to use de-identified data from CARDIA for this study was provided by the Institutional Review Board at Vanderbilt University Medical Center (IRB #211402). CCHC was approved by the Committee for the Protection of Human Subjects (CPHS) at the University of Texas Health Sciences Center at Houston (IRB #HSC-SPH-03-007-B—CCHC). UK Biobank access was approved under proposal #57492. SteatoSITE was approved by the West of Scotland Research Ethics Committee 4 (Reference: 20/WS/0002) and Public Benefit and Privacy Panel for Health and Social Care (Reference: 1819-0091).

### Example 8: Data and Code

Proteomic data used in these analyses are publicly available through the following sources: Coronary Artery Risk Development in Young Adults study (CARDIA; cardia.dopm.uab.edu) or via the dbGaP (dbGaP identifier phs003491.v1.p1); UK Biobank data is available at UK Biobank Research Access Portal (analyses here conducted under proposal approval 57492); Cameron County Hispanic Cohort data is available from the Coordinating Center (sph.uth.edu/research/centers/hispanic-health/). RNA-sequencing data used in these analyses are available from the following sources: SteatoSITE (bulk RNA-seq) data are deposited in the European Nucleotide Archive (www.ebi.ac.uk/ena; study accession number: PRJEB58625) and have been previously published⁹. Data from the human spatial and single cell liver atlases are available at www.livercellatlas.org and have been published³⁴.

Statistical code used in this study can be found at github.com/asperry125/MASLD (DOI: 10.5281/zenodo.13891944) and github.com/Banovich-Lab/MASLD (DOI: 10.5281/zenodo.13899757).

### Example 9: Hepatic Steatosis Assessment

In CARDIA, hepatic steatosis was measured as liver attenuation on computed tomography as previously described, where lower levels of liver attenuation are associated with greater steatosis¹⁰. MASLD was defined as liver attenuation <40 Hounsfield units. In the UK Biobank, hepatic steatosis was measured by magnetic resonance imaging in a subset of participants at instance 2 using the iterative decomposition of water and fat with echo asymmetric and least-squares estimation (IDEAL) protocol, as previously described⁸⁵. MASLD was defined as a proton density fat fraction >5.5%⁸⁶. In CCHC, vibration-controlled transient elastography was used to measure CAP (FibroScan 502 Touch or FibroScan 530 Compact, Echosens; automatic probe selection) for 10 valid measures with the median used in analysis, as described⁸⁷.

### Example 10: Proteomics

CARDIA: Quantification of the circulating proteome was performed using aptamer-based technology (Somalogic, Boulder, CO) which measured 7524 aptamers. Details on the technical aspects (including target specificity^{88,89}) and variability of this platform have been previously published⁹⁰⁻⁹². Sixty-eight participants had >1 samples measured and their proteomic data was averaged for analysis. Non-human aptamers (N=233) and aptamers with a coefficient of variation >20% (N=61) were excluded. Testing was conducted for batch effect and participant outliers using principal component analysis and identified neither. Proteins were log-transformed and standardized (mean 0, variance 1) prior to use in models.

UK Biobank: Recently released proteomic data from the Olink Explore platform (Olink, Uppsala, Sweden) measured from the instance 0 visit were used in this study⁸². Technical considerations on the Olink assay are published elsewhere⁹³. Variability of Olink proteomics measurements in UK Biobank have been reported⁸², with a median CV of 6%. Of the 1463 proteins measured, 130 proteins were excluded where >40% of reported measurements were below the limit of detection and another 3 proteins were excluded where >20% of reported measurements were missing. Proteins were standardized (mean 0, variance 1) prior to use in models.

CCHC: Proteomics were performed in CCHC participants using the Olink Explore 1536 platform. Proteins were standardized (mean 0, variance 1) prior to use in models.

### Example 11: Spatial and Integrated Single-Nuclear and Single-Cell Transcriptomics

To assess cell-specific and spatial expression patterns of implicated protein targets, integrated single-cell and single-nuclear RNA-sequencing (N=19; single-cell RNA-seq, N=14; single-nuclear RNA-seq, N=5; fatty=7; non-fatty=11; unknown=1) and Visium spatial transcriptomics data (5 tissue slices from 4 individuals were harnessed, of which 2 had steatosis and 2 did not) previously published from the collaborative group³⁴. Expression patterns of implicated proteins were assessed by mapping significant model proteins to their corresponding gene symbol including genes that were expressed in both the integrated single-cell and single-nuclear RNA-seq and spatial transcriptional datasets, resulting in total of 198 genes represented across the three datasets (FIG. 10). These genes were assessed by single gene expression measures as well as an expression composite score that represents the transcriptional signature of all model genes in each individual cell (integrated single-cell and single-nuclear RNA-seq) or spot (Visium data). This expression composite score was generated using the AddModuleScore function (implemented in Seurat v5.0.0). To identify differential expression of nominated targets in the liver healthy samples were compared to early steatotic samples using Visium data where both healthy and early steatotic samples were available. Differentially expressed genes were assessed using negative binomial model implemented in the FindMarkers function (Seurat). Only target genes expressed in at least 10% of the spots were included in the analysis (198 genes) for differential expression. Differential expression was defined as FDR adjusted P<0.05 and |log₂ fold change|>0.25 and a minimum difference in expressed spots >10% between fatty and non-fatty. The effect size estimates of the differential expression analysis was confirmed via negative binomial mixed models with sample as random effect (generalized linear mixed models are more sensitive to the dispersion in single-cell data compared to generalized linear models), with high agreement between log₂ fold change and the negative binomial mixed model coefficients for all 198 model targets (Pearson r=0.86) and for the 33 differentially expressed genes (Pearson r=0.99; FIG. 13).

### Example 12: Transcriptional Differences Across Histologically-Defined MASLD Stages

The pattern of expression was explored across the 33 genes prioritized by the spatial data analysis above (33 differentially expressed genes between healthy and steatotic tissue out of 198 genes tested, see Results) in the SteatoSITE biopsy cohort (34 controls, 499 with MASLD) categorized based on NAFLD activity score (NAS) for steatosis (only those samples with scores 1, 2 and 3 were chosen) and compared to control samples⁹. For FIG. 14A-14B, those individuals with NASH-CRN stage F4 fibrosis (N=74) were excluded, given differences in expression patterns detected in the initial study⁹ and differences in physiology with advanced fibrosis (including paradoxical loss of hepatic fat⁹⁴). Reads were normalized using the weighted trimmed mean of M values method⁹⁵. Differential gene expression analysis was performed using limma-voom (v3.28.14) with the protein-coding genes using an FDR of 5% (Benjamini-Hochberg)⁹⁶. Of the 33 genes passed forward for assessment in bulk transcriptomics, 12 were not significantly expressed in any of the stages of steatosis (by adjusted P<0.05) and were not included in visualization. For FIG. 15, participants across all stages of NASH-CRN stage were included.

### Example 13: Humanized Liver-On-a-Chip MASLD Model

The goal of “liver-on-a-chip” technology is to simulate the liver microenvironment which retains key characteristics of native liver function over long-term in culture. The quad culture was set up following the manufacturer's protocol. The methods below are reproduced from recent work⁹⁷ directly for rigor and reproducibility, and this citation provides scientific attribution for this. Briefly, by design, each polydimethylsiloxane (PDMS) chip (Chip-Si; Emulate) includes hepatocytes (Gibco) in the apical channel and non-parenchymal cells [NPCs: Kupffer (SAMSARA Science), Stellate (iXCells Biotechnologies) and Liver Sinusoidal Endothelial Cells (LSECs; Cell Systems)] in the basal channel (Table 10). These two channels are separated by a porous membrane, coated by hepatic extracellular matrix (ECM). This setting allows the cell-to-cell interaction mimicking the in vivo system. The top channel was seeded with hepatocytes at a concentration of 3.5×10⁶ cells/mL, followed by overlay with matrigel on the next day. The day after hepatocyte overlay, cell suspensions of three NPCs were mixed in a 1:1:1 ratio (v/v/v) to generate the bottom channel tri-cell mixture. The final seeding density of each cell types in the bottom channel were: LSECs: 3×10⁶ cells/mL; Stellate cells: 0.1×10⁶ cells/mL; Kupffer cells: 0.5×10⁶ cells/mL. Chips were maintained for another 96 hours at this condition before treating with fatty acids (FAs). An early phase of MASLD was mimicked by treating both channels of the LOC either with vehicle control (1% BSA) or a combination of FAs (oleic acid 300 μM: 300 μM palmitic acid bound with 1% BSA) for 5 consecutive days.

TABLE 10
Cells used for liver-on-a-chip quad culture.

| | | Catalogue | |
|---|---|---|---|
| Cell Names | Company | No. | Source |
| Cryo Human | Gibco | HU8305 | Normal human |
| Hepatocytes | | | donor |
| Primary Human Liver | Cell Systems | ACBRI | Normal human |
| Sinusoidal MVEC | | 566 | donor |
| Human Liver Kupffer | SAMSARA | HLKC | Normal human |
| Cells | Science | | donor |
| Human Hepatic | iXCells | 10HU-210 | Normal human |
| Stellate Cells | Biotechnologies | | donor |

Hepatocytes and NPCs treated with either vehicle control or FAs were imaged directly under brightfield microscope (BioRad). Chips were fixed with 4% paraformaldehyde (4% PFA) followed by permeabilization of both channels with 0.1% Triton® X-100 before staining. Permeabilized cells in both channels were incubated with LipidSpot™ for 10 minutes. The chips were examined under a fluorescence microscope (ECHO Revolve microscope).

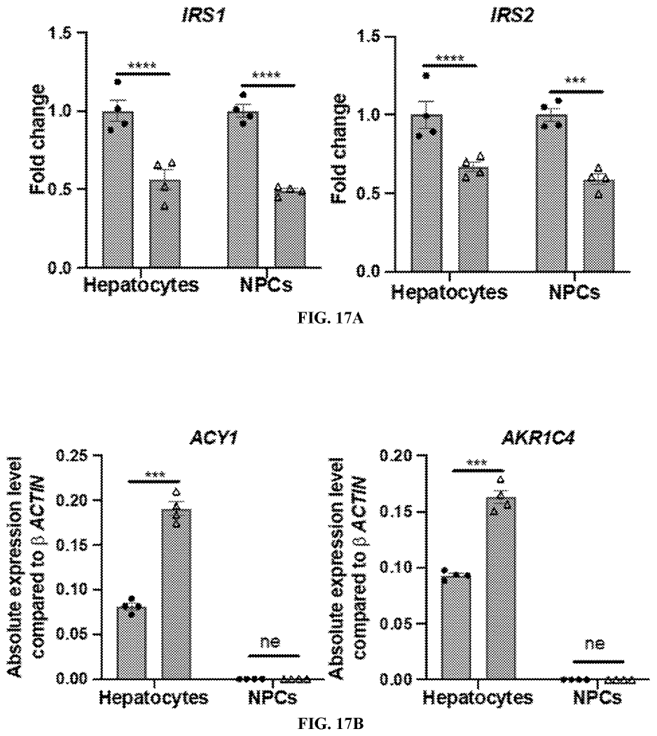

Recent methods for these experiments were followed with minimal alterations of that text for purposes of scientific reproducibility (and citation provided⁹⁷). After 5 days of dosing with FAs, the chips were disconnected, washed with 1×PBS, and filled with RNAlater (Invitrogen) to preserve cells for RNA extraction (PureLink RNA Mini Kit, Thermo Fischer Scientific). Total RNA was eluted in 20 μL, treated with DNAse, and applied RNA Clean & Concentrator-5 with DNase I (Zymo Research) following manufacturer's protocol. RNA concentration was quantified by spectrophotometry (Nanodrop 2000, Thermo Fischer Scientific), and performed cDNA synthesis (High-Capacity cDNA Reverse Transcription Kit, Thermo Fischer Scientific). PCR was performed to quantify select genes (HMGCS1, SERPINE1, HSPA1B, ENO3, HSPA1A, PYGL, CDA, SHBG, IL1RAP, IGFBP2, ME1, CTSZ, DEFB1, IRS1, IRS2, FABP4, SREBP1c, PPARα, PPARγ, ACY1, AKR1C4, β-ACTIN; via ExiLENT SYBR® Green master mix, Exiqon, Vedbok; Quant Studio 6 Flex Real-Time PCR System) up to 40 amplification cycles, with any amplification cycle (Ct) greater than or equal to 40 assigned as a “negative” threshold, indicating that corresponding genes were not expressed above the limit of detection of the PCR assay (and therefore not included in the calculations). For FIG. 16E absolute gene expression was quantified by 2^{-ΔCt} method after normalization of genes of interest to the internal control β-ACTIN, whereas relative gene expression was used for FIG. 16D and FIG. 17A. All qRT-PCR primer sequences are summarized in Table 11.

TABLE 11
Primer list for liver-on-a-chip experiments.

| | | | SEQ |
|---|---|---|---|
| | | | ID |
| Sl. No. | Gene Names | Sequences (5→3) | NO: |
| 1. | HMGCS1 F | AAGTCACACAAGATGCTACACCG | 22 |
| | HMGCS1 R | TCAGCGAAGACATCTGGTGCC | 23 |
| 2. | SERPINE1 F | CTCATCAGCCACTGGAAAGGCA | 24 |
| | SERPINE1 R | GACTCGTGAAGTCAGCCTGAAA | 25 |
| 3. | HSPA1B F | ACCTTCGACGTGTCCATCCTGA | 26 |
| | HSPA1B R | TCCTCCACGAAGTGGTTCACCA | 27 |
| 4. | ENO3 F | TGGGAAGGATGCCACCAATGTG | 28 |
| | ENO3 R | GCGATAGAACTCAGATGCTGCC | 29 |
| 5. | HSPA1A F | ACCTTCGACGTGTCCATCCTGA | 30 |
| | HSPA1A R | TCCTCCACGAAGTGGTTCACCA | 31 |
| 6. | ME1 F | GGAGTTGCTCTTGGTGTTGTGG | 32 |
| | ME1 R | GGATAAAGCCGACCCTCTTCCA | 33 |
| 7. | CTSZ F | GGATGGTGTCAACTATGCCAGC | 34 |
| | CTSZ R | CACGCTCCCTTCCTCTTGATGT | 35 |
| 8. | DEFB1 F | GGTAACTTTCTCACAGGCCTTGG | 36 |
| | DEFB1 R | TCCCTCTGTAACAGGTGCCTTG | 37 |
| 9 | PYGL F | CACTTCAGTGGCAGATGTGGTG | 38 |
| | PYGL R | GCAGTGGAAATCTGCTCTGACAG | 39 |
| 10 | CDA F | GCCAAGAAGTCAGCCTACTGCC | 40 |
| | CDA R | CTTCTGGATAGCGGTCCGTTCA | 41 |
| 11. | IL1RAP F | CTGAGGATCTCAAGCGCAGCTA | 42 |
| | IL1RAP R | AGCAGGACTGTGGCTCCAAAAC | 43 |
| 12. | SHBG F | CAGGACAAGAGCCTATCGCTGT | 44 |
| | SHBG R | GTCATCCTTAGGGTTGGTATCCC | 45 |
| 13. | IRSIF | AGTCTGTCGTCCAGTAGCACCA | 46 |
| | IRS1 R | ACTGGAGCCATACTCATCCGAG | 47 |
| 14. | IRS2 F | CCTGCCCCCTGCCAACACCT | 48 |
| | IRS2 R | TGTGACATCCTGGTGATAAAGCC | 49 |
| 15. | PPARα F | TCGGCGAGGATAGTTCTGGAAG | 50 |
| | PPARα R | GACCACAGGATAAGTCACCGAG | 51 |
| 16. | PPARγ F | AGCCTGCGAAAGCCTTTTGGTG | 52 |
| | PPARγ R | GGCTTCACATTCAGCAAACCTGG | 53 |
| 17. | FABP4 F | ACGAGAGGATGATAAACTGGTGG | 54 |
| | FABP4 R | GCGAACTTCAGTCCAGGTCAA | 55 |
| 18. | SREBP1c F | ACTTCTGGAGGCATCGCAAGCA | 56 |
| | SREBP1cR | AGGTTCCAGAGGAGGCTACAAG | 57 |
| 19. | β-ACTIN F | CACCATTGGCAATGAGCGGTTC | 58 |
| | β-ACTIN R | AGGTCTTTGCGGATGTCCACGT | 59 |
| 20. | IGFBP2 F | CGAGGGCACTTGTGAGAAGCG | 60 |
| | IGFBP2 R | TGTTCATGGTGCTGTCCACGTG | 61 |
| 21. | ACY1 F | CCTACACTCTCCTCCATCTTGC | 62 |
| | ACY1 R | CCTGGCATAGATGTAGCCCTCA | 63 |
| 22. | AKR1C4 F | CCGAAGCAAGATTGCAGATGGC | 64 |
| | AKR1C4 R | GTGAGCTTTCCAAGGCTGGTTG | 65 |

The proteins were prioritized that corresponded to the candidate genes which were increased at mRNA level, from two data sets. The first set was chosen from candidate gene targets identified across both proteomic and spatial transcriptomics, were transcriptionally increased in the LOC model, and had commercially available validated ELISA assays (HMGCS1, SERPINE1, PYGL, CTSZ, and DEFB1). The second set, chosen from the CARDIA proteomic data set, were highly associated with liver steatosis, had high confidence scores for extracellular expression (see Table 8, and had commercially available validated ELISA assays (ACY1 and AKR1C4).

Effluents and inlet media were collected from all Pod outlet and inlet reservoirs respectively of Hepatocyte and NPC channels, avoiding direct contact with reservoir “Vias”, stored in pre-labeled appropriate tubes, and placed on ice immediately. The concentration of secretory cellular proteins (SERPINE1 [Abcam, Cambridge, UK], HMGCS1, PYGL, CTSZ, DEFB1, ACY1, and AKR1C4 [LSBio, WA, USA]) in the sample effluents of different groups (Control and FA-treated) was quantified via ELISA as described in the manufacturers' protocols (expressed as either pg/mL or ng/mL cellular effluent media; FIG. 16E). Background/Inlet media-subtracted data values are represented in the graphs. For SERPINE1, samples were run at 1:50 dilution in order to obtain optimal dilution that produced an OD reading (at 450 nm) within the OD range of the positive control standard dilution series, and the represented concentration read from the standard curve was multiplied by the dilution factor. Both DEFB1 and CTSZ were not within the detectable range for hepatocyte channel, hence not included in the calculation. Conversely, expression of ACY1 and AKR1C4 were checked in hepatocytes channel only. Raw values of individual chip effluent media including standards are summarized in Table 9.

### Example 14: Quantification and Statistical Analysis; Relating the Circulating Proteome to Hepatic Steatosis to Identify Biological Pathways of Steatosis and Development of a Biomarker Panel (“MASLD Score”)

Relations of individual aptamers with hepatic steatosis were examined via linear regression with aptamers as the predictors adjusted for age, gender, race, and BMI with a false discovery rate of 5% (Benjamini-Hochberg) in the CARDIA cohort using a derivation (70%) and validation (30%) split design balanced on CT liver attenuation. To generate a multivariable protein score of hepatic steatosis (referred to as “MASLD score”), least absolute shrinkage and selection operator (LASSO) with non-penalized adjustments for age, gender, race, and BMI was used in the CARDIA derivation sample. A participant's MASLD score was calculated by taking the sum of the products of protein levels and coefficients from the LASSO model (MASLD score=β_{protein1 from model}*protein 1+β_{protein2 from model}*protein 2+ . . . +β_{protein n from model}*protein n). Notably, the coefficients on age, gender, race, and BMI are not included in the MASLD score, and reflects proteomic associations conditioned on BMI. Model fit was examined in both CARDIA derivation and validation samples. This MASLD score was then recalibrated for use in UK Biobank and CCHC (which used Olink proteomics platforms in contrast to CARDIA, which used a SomaScan platform), using LASSO regression with the original MASLD score as the dependent variable and all overlapping proteins (matching between the Olink and SomaScan platforms on UniProt identifier) as the independent variables. To create a clinically translatable MASLD score, the proteins included in the MASLD score were ranked by the absolute value of the beta coefficients and used the top 21 proteins. 21 proteins were chosen because Olink currently offers a customizable platform of up to 21 proteins with absolute quantification.

Pathway analysis was performed on the 237 unique proteins significant in both CARDIA derivation and validation subsamples (at a 5% FDR) using ClusterProfiler⁹⁸ in R on the KEGG and Reactome databases. Hypergeometric tests were used to evaluate enrichment level for each pathway specifying proteins that matched between the SomaScan platform and the reference as background. The top 10 most enriched pathways in both KEGG and Reactome were visualized together via lollipop plots. To identify hub genes, protein-protein interaction scores for 235 out of the 237 total significantly differentially expressed protein genes were retrieved from the STRING database²⁴ (based on genes present in STRING). Hub genes were determined as any protein with more than 5 high-confidence interactions (interaction score>700). Genes assigned as hub genes and all interactions were visualized using Cytoscape⁹⁹.

For transcriptional enrichment in the liver, tissue-specific gene expression enrichment of the transcripts corresponding to the 237 unique proteins associated with hepatic steatosis was performed by R package TissueEnrich¹⁰⁰ using a hypergeometric test based on tissue expression patterns in Human Protein Atlas database¹⁰¹. For proteomic enrichment in the liver, TissueEnrich was used, leveraging the reference proteomic dataset from the Genotype-Tissue Expression (GTEx) Project¹⁰², which had been generated in 32 normal human tissues using tandem mass tag (TMT) 10 plex/MS3 quantitative mass spectrometry. The background set for each analysis consisted of the proteins on the SomaScan platform that were found in the respective reference.

### Example 15: Quantification and Statistical Analysis; Testing the Association Between a Protein Score of MASLD with Development of MASLD and Clinical Outcomes

In UK Biobank, Cox regression was used to examine the relation of the MASLD score with clinical endpoints. Death and type of death (cardiovascular death, cancer death) were defined by using death registry data (UK Biobank Data Field 40000) in conjunction with the primary cause of death International Classification of Disease (ICD) 10 code (UK Biobank Data Field 40001). Translating ICD10 codes to type of death was conducted as previously reported¹⁰³. Censoring for clinical endpoints was determined by region-specific censor dates for each participant based on the location of initial assessment (UK Biobank Data Field 54). Censoring for death outcomes was 30 Nov. 2022 for all alive participants. Cause-specific death models were compared against the Fine-Gray subdistribution hazard model. Non-death outcomes in UK Biobank were defined by ICD10 diagnosis codes grouped into relevant “phecodes” via the PheWAS package¹⁰⁴. For each phecode, a case, control, and excluded status was generated for each subject. Time to event for phecodes was defined as the date of the earliest relevant ICD10 was documented. Prevalent conditions were defined by self-report or physician diagnosis (Data Fields 20002, 2443, 6150). Censoring for incident phecodes (e.g., diabetes) was determined by region-specific censoring dates or the date of death for non-event participants. Sequential models with increasing adjustments were created 1) unadjusted 2) age, gender, race, BMI 3) age, gender, race, BMI, Townsend Deprivation Index, diabetes, smoking, alcohol use, systolic blood pressure, and LDL. A sensitivity analysis was conducted including further adjustment for AST, ALT, and hemoglobin Alc.

Adjusted models (age, gender, race, BMI, diabetes [removed from models for diabetes], smoking, alcohol use, systolic blood pressure, LDL) were compared with and without the MASLD score to compare differences in C-statistics and net reclassification index (NRI; calculated at the 75th percentile of follow-up time for events).

### Example 16: Quantification and Statistical Analysis; Differential Expression Analysis Across Stages of Steatosis and in a Humanized “Liver-On-Chip” Model

Differential gene expression analysis across stages of steatosis (FIG. 14), was performed using limma-voom with the protein-coding genes using an FDR of 5% (Benjamini-Hochberg; * p<0.05, ** p<0.01, *** p<0.001, **** p<0.0001). Results from the “liver-on-chip” experiments were analyzed by an unpaired t test and expressed as mean±standard error of 3 independent experiments (FIG. 16, * p<0.05, ** p<0.01, *** p<0.001, **** p<0.0001).

### Example 17: Discussion of Examples

The goal of the present study was to provide a translational resource that integrates insights from the human proteome and liver tissue transcriptome with clinically accessible hepatic phenotypes across multiple diverse, large human cohorts to identify and prioritize targets for downstream study. Given recent data highlighting the utility of human proteomic discovery “focused” by transcriptional profiling in human tissue⁵⁷⁻⁵⁹, targets identified from proteomic associations were mapped at epidemiologic scale to human liver tissue (at single cell and spatial resolution), including in dynamic models of induced steatosis (liver-on-a-chip). The study was focused on an at-risk population (rather than those with established MASLD) due to the desire to identify early markers of disease in a broad, diverse group. The principal findings of this integrated approach were identification of physiologically plausible, reproducible proteomic correlates of imaging-based measures of hepatic steatosis that (1) were related to key outcomes in >26000 individuals (including fatty liver disease); (2) implicated broad pathways of metabolism, inflammation, and fibrosis, with predominant enrichment in human liver at the RNA and protein levels; (3) co-localized at the RNA level to areas of steatosis histologically via spatial and bulk transcriptional studies, several with concordant proteomic and transcriptional effects; (4) were expressed at the protein and RNA level in a dynamic, cell-specific fashion in human liver-on-a-chip during early steatosis induction. This fully translational approach provides a powerful discovery resource that links populations to tissue to dynamic hepatic cellular states, pinpointing targets for downstream genetic and experimental studies.

A key innovation in the current approach is the tiered design strategy to prioritize biomarkers across proteomic, transcriptional, and in vitro model systems-all within humans. This approach was based on the high enrichment of transcript expression of genes encoding the MASLD proteome in the liver (beyond any other tissue). Moreover, identified protein targets specified broad pathways central to MASLD, including regulation of hepatocyte regeneration (EGFR²⁵), injury, apoptosis (MET²⁷), inflammation (CXCL2²⁸, CRP, SERPINE1), metabolism (ACY1^{8,12}, FAH¹⁶, ADH1A^{14,15}, ALDOB, SORD¹⁶, AKR1D1¹⁷, AKR1C4^{18,89}), and fibrosis (IGF-1²⁶). Several findings were consistent with results from murine studies (e.g., ALDOB, PIGR, VTN, AFM⁶⁰), suggesting shared biology.

Given that these tissue references are from “normal” tissue banks at bulk resolution (e.g., Human Protein Atlas/GTEx), the MASLD-associated proteomic targets were further explored at single nuclear and spatial resolution in human liver at an early MASLD stage³⁴. A key finding from this approach was the striking concordance of the circulating proteomic effect size in the large, population-based cohort (CARDIA) and the fold-difference between healthy and fatty liver. Plasma proteins more abundant in patients with lower degrees of hepatic steatosis corresponded to higher mRNA expression in non-steatotic livers (and vice versa). Gene activity (as defined by a gene expression score across 198 steatosis-associated proteins) mapped primarily to histopathologic areas of steatosis, with a predominant hepatocyte expression and zonation pattern. Interestingly, a greater expression of CTSZ was also observed in macrophages and both migratory and conventional dendritic cells, consistent with recent reports linking inflammatory cells to MASLD pathogenesis in a murine model⁶¹. Several genes differentially expressed in spatial transcription were dynamic across MASLD stages, showing consistent directionality with spatial transcriptomic and circulating proteomics⁹, which further supports validity (and prioritization) of these targets. Notably, recent innovative efforts to map a circulating snapshot of metabolic biology (via the human proteome) into hepatic transcriptional states have been successful, albeit in a small sample with high metabolic disease prevalence⁵⁸. Indeed, the value of transcriptional indexing of the human proteome across broad at-risk populations has recently been highlighted⁵⁷.

A second key innovation of this approach was the inclusion of a humanized LOC steatosis platform that allowed us to link induction of steatosis in the liver to changes in mRNA transcripts and secreted protein levels prioritized by human proteomic and transcriptional studies. While the LOC model used here has been validated to recapitulate key aspects of human liver physiology⁶²⁻⁶⁴, it has mostly been used as a drug screen for hepatotoxicity⁶⁵⁻⁶⁸, though its use in modeling steatosis is emerging⁶⁹. The model deployed here included both hepatocytes and non-parenchymal cells (Kupffer cells, stellate cells, and endothelial cells) subject to treatment with a cocktail of fatty acids. While this model is admittedly less complex than human MASLD, it replicated key histological and transcriptional features of human MASLD, allowing us to query for direct changes in response to steatosis induction in mRNA transcripts in both hepatocyte and non-hepatocyte cell types derived from human studies. The model discriminated secreted proteins from hepatocyte-derived vs. non-hepatocyte cells: for example, CTSZ was increased in the non-hepatocyte cell effluent but was below the limit of detection in the hepatocyte channel, corresponding to its mRNA expression pattern. These data fundamentally complement the human circulating proteomic and transcriptional studies by linking transcriptional and proteomic changes in distinct hepatic cell types during early steatosis to a circulating proteome of MASLD susceptibility in human populations. While a full exposition of the different targets from the RNA-seq and liver-on-a-chip data is outside the scope of this report, these results from the spatial, single cell, and chip data provide insights into localization of proteomic targets from circulation at the RNA level to areas of steatosis and their dynamicity during steatosis (difficult to obtain with human biopsy alone). Further insights into mechanism will require in-depth gain- and loss-of-function in model systems, organoids, or organ-on-a-chip platforms, predicated on targets identified here and in other systems.

An important point in the study design was the use of imaging as opposed to tissue biopsy. While contemporary approaches to molecular discovery in steatosis have examined associations with biopsy phenotypes in established disease^{7,8,58}, these approaches are not possible at the large epidemiologic scale required for molecular discovery in individuals with a lower risk profile. In addition, it is important to point out that modem risk assessments do not necessarily require liver biopsy to diagnose MASLD (defined by presence of hepatic steatosis, which is easily detected radiologically, in presence of one or more cardiometabolic risk factors)⁷⁰. The robustness of the results across MRI, CT, and ultrasound-based methods, each of which has been used in large studies to stratify degree of steatosis⁷¹, lends credibility. It would be anticipated that ascertainment bias or misclassification by any of these imaging measures to increase noise (variance) rather than cause false discovery, thereby reducing external reproducibility/validity. In addition, the cohorts—drawn from a population rather than secondary care setting—are unlikely to be biopsied in routine practice, rendering an absence of “early MASLD” cohorts that are both biopsy-defined and of sufficient scale and duration to guide proteomic discovery and correlate these findings with clinical outcomes. Finally, the results were validated against human tissue data (GTEx tissue proteomics and spatial and single cell data) and dynamic (liver-on-a-chip) systems, all of which showed concordant results. Future studies in earlier populations may be warranted to continue to build on these results.

In conclusion, across ≈5000 participants with clinical, imaging, and biochemical data, a proteomic architecture of hepatic steatosis is defined with replication across a MASLD spectrum (from early- to high-risk metabolic cohorts) and strong association with MASLD-related disease in >26000 individuals (with clinical prognostication retained down to a 21-protein panel). Proteins implicated by these population-based approaches were highly enriched at a transcriptional level in human liver and specified canonical pathways of steatosis in addition to other plausible pathways. Spatially enriched activity of these genes was observed in areas of steatosis, with concordance between the circulating proteomic effects on liver fat and the fold differences between healthy and fatty liver by spatial transcription. Several targets additionally demonstrated concordant changes during evolution of steatosis across histologically defined stages and within a humanized “liver-on-a-chip” model system at a transcriptional and proteomic level, linking clinical, proteomic, transcriptional, and human system perturbation results. These results contextualize the promise of multi-level discovery—across broad clinical populations, proteome, and tissue studies—to discern biologically relevant, spatially enriched targets in MASLD for downstream mechanistic, diagnostic, and prognostic work.

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

1. Riazi, K., Azhari, H., Charette, J. H., Underwood, F. E., King, J. A., Afshar, E. E., Swain, M. G., Congly, S. E., Kaplan, G. G., and Shaheen, A. A. (2022). The prevalence and incidence of NAFLD worldwide: a systematic review and meta-analysis. Lancet Gastroenterol Hepatol 7, 851-861. 10.1016/S2468-1253(22)00165-0.
2. Mantovani, A., Petracca, G., Beatrice, G., Csermely, A., Tilg, H., Byrne, C. D., and Targher, G. (2022). Non-alcoholic fatty liver disease and increased risk of incident extrahepatic cancers: a meta-analysis of observational cohort studies. Gut 71, 778-788. 10.1136/gutjnl-2021-324191.
3. Meyersohn, N. M., Mayrhofer, T., Corey, K. E., Bittner, D. O., Staziaki, P. V., Szilveszter, B., Hallett, T., Lu, M. T., Puchner, S. B., Simon, T. G., et al. (2021). Association of Hepatic Steatosis With Major Adverse Cardiovascular Events, Independent of Coronary Artery Disease. Clin Gastroenterol Hepatol 19, 1480-1488 e1414. 10.1016/j.cgh.2020.07.030.
4. Anstee, Q. M., Reeves, H. L., Kotsiliti, E., Govaere, O., and Heikenwalder, M. (2019). From NASH to HCC: current concepts and future challenges. Nat Rev Gastroenterol Hepatol 16, 411-428. 10.1038/s41575-019-0145-7.
5. Romeo, S., Kozlitina, J., Xing, C., Pertsemlidis, A., Cox, D., Pennacchio, L. A., Boerwinkle, E., Cohen, J. C., and Hobbs, H. H. (2008). Genetic variation in PNPLA3 confers susceptibility to nonalcoholic fatty liver disease. Nature genetics 40, 1461-1465. 10.1038/ng.257.
6. Luo, Y., Wadhawan, S., Greenfield, A., Decato, B. E., Oseini, A. M., Collen, R., Shevell, D. E., Thompson, J., Jarai, G., Charles, E. D., and Sanyal, A. J. (2021). SOMAscan Proteomics Identifies Serum Biomarkers Associated With Liver Fibrosis in Patients With NASH. Hepatol Commun 5, 760-773. 10.1002/hep4.1670.
7. Corey, K. E., Pitts, R., Lai, M., Loureiro, J., Masia, R., Osganian, S. A., Gustafson, J. L., Hutter, M. M., Gee, D. W., Meireles, OR., et al. (2022). ADAMTSL2 protein and a soluble biomarker signature identify at-risk non-alcoholic steatohepatitis and fibrosis in adults with NAFLD. Journal of hepatology 76, 25-33. 10.1016/j.jhep.2021.09.026.
8. Sanyal, A. J., Williams, S. A., Lavine, J. E., Neuschwander-Tetri, B. A., Alexander, L., Ostroff, R., Biegel, H., Kowdley, K. V., Chalasani, N., Dasarathy, S., et al. (2023). Defining the serum proteomic signature of hepatic steatosis, inflammation, ballooning and fibrosis in non-alcoholic fatty liver disease. J Hepatol 78, 693-703. 10.1016/j.jhep.2022.11.029.
9. Kendall, T. J., Jimenez-Ramos, M., Turner, F., Ramachandran, P., Minnier, J., McColgan, M. D., Alam, M., Ellis, H., Dunbar, D. R., Kohnen, G., et al. (2023). An integrated gene-to-outcome multimodal database for metabolic dysfunction-associated steatotic liver disease. Nat Med 29, 2939-2953. 10.1038/s41591-023-02602-2.
10. VanWagner, L. B., Wilcox, J. E., Ning, H., Lewis, C. E., Carr, J. J., Rinella, M. E., Shah, S. J., Lima, J. A. C., and Lloyd-Jones, D. M. (2020). Longitudinal Association of Non-Alcoholic Fatty Liver Disease With Changes in Myocardial Structure and Function: The CARDIA Study. J Am Heart Assoc 9, e014279. 10.1161/JAHA.119.014279.
11. Rinella, M. E., Lazarus, J. V., Ratziu, V., Francque, S. M., Sanyal, A. J., Kanwal, F., Romero, D., Abdelmalek, M. F., Anstee, Q. M., Arab, J. P., et al. (2023). A multisociety Delphi consensus statement on new fatty liver disease nomenclature. Hepatology 78, 1966-1986. 10.1097/hep.0000000000000520.
12. Pirola, C. J., and Sookoian, S. (2018). Multiomics biomarkers for the prediction of nonalcoholic fatty liver disease severity. World journal of gastroenterology. WJG 24, 1601-1615. 10.3748/wjg.v24.i15.1601.
13. Ma, J., Tan, X., Kwon, Y., Delgado, E. R., Zarnegar, A., DeFrances, M. C., Duncan, A. W., and Zarnegar, R. (2022). A Novel Humanized Model of NASH and Its Treatment With META4, A Potent Agonist of MET. Cell Mol Gastroenterol Hepatol 13, 565-582. 10.1016/j.jcmgh.2021.10.007.
14. Li, H., Toth, E., and Cherrington, N. J. (2018). Alcohol Metabolism in the Progression of Human Nonalcoholic Steatohepatitis. Toxicol Sci 164, 428-438. 10.1093/toxsci/kfy106.
15. Aljomah, G., Baker, S. S., Liu, W., Kozielski, R., Oluwole, J., Lupu, B., Baker, R. D., and Zhu, L. (2015). Induction of CYP2E1 in non-alcoholic fatty liver diseases. Exp Mol Pathol 99, 677-681. 10.1016/j.yexmp.2015.11.008.
16. Niu, L., Geyer, P. E., Wewer Albrechtsen, N. J., Gluud, L. L., Santos, A., Doll, S., Treit, P. V., Holst, J. J., Knop, F. K., Vilsboll, T., et al. (2019). Plasma proteome profiling discovers novel proteins associated with non-alcoholic fatty liver disease. Mol Syst Biol 15, e8793. 10.15252/msb.20188793.
17. Gathercole, L. L., Nikolaou, N., Harris, S. E., Arvaniti, A., Poolman, T. M., Hazlehurst, J. M., Kratschmar, D. V., Todorcevic, M., Moolla, A., Dempster, N., et al. (2022). AKR1D1 knockout mice develop a sex-dependent metabolic phenotype. J Endocrinol 253, 97-113. 10.1530/JOE-21-0280.
18. Zeng, C. M., Chang, L. L., Ying, M. D., Cao, J., He, Q. J., Zhu, H., and Yang, B. (2017). Aldo-Keto Reductase AKR1C1-AKR1C4: Functions, Regulation, and Intervention for Anti-cancer Therapy. Front Pharmacol 8, 119. 10.3389/fphar.2017.00119.
19. Lyall, M. J., Cartier, J., Thomson, J. P., Cameron, K., Meseguer-Ripolles, J., O'Duibhir, E., Szkolnicka, D., Villarin, B. L., Wang, Y., Blanco, G. R., et al. (2018). Modelling non-alcoholic fatty liver disease in human hepatocyte-like cells. Philos Trans R Soc Lond B Biol Sci 373. 10.1098/rstb.2017.0362.
20. Gorce, M., Lebigot, E., Arion, A., Brassier, A., Cano, A., De Lonlay, P., Feillet, F., Gay, C., Labarthe, F., Nassogne, M. C., et al. (2022). Fructose-1,6-bisphosphatase deficiency causes fatty liver disease and requires long-term hepatic follow-up. J Inherit Metab Dis 45, 215-222. 10.1002/jimd.12452.
21. Coles, B. F., and Kadlubar, F. F. (2005). Human alpha class glutathione S-transferases: genetic polymorphism, expression, and susceptibility to disease. Methods Enzymol 401, 9-42. 10.1016/S0076-6879(05)01002-5.
22. Nagamani, S. C., Erez, A., and Lee, B. (2012). Argininosuccinate lyase deficiency. Genet Med 14, 501-507. 10.1038/gim.2011.1.
23. Zhang, T., Zhang, N., Xing, J., Zhang, S., Chen, Y., Xu, D., and Gu, J. (2023). UDP-glucuronate metabolism controls RIPK1-driven liver damage in nonalcoholic steatohepatitis. Nat Commun 14, 2715. 10.1038/s41467-023-38371-2.
24. Szklarczyk, D., Kirsch, R., Koutrouli, M., Nastou, K., Mehryary, F., Hachilif, R., Gable, A. L., Fang, T., Doncheva, N. T., Pyysalo, S., et al. (2023). The STRING database in 2023: protein-protein association networks and functional enrichment analyses for any sequenced genome of interest. Nucleic Acids Res 51, D638-D646. 10.1093/nar/gkac1000.
25. Bhushan, B., Banerjee, S., Paranjpe, S., Koral, K., Mars, W. M., Stoops, J. W., Orr, A., Bowen, W. C., Locker, J., and Michalopoulos, G. K. (2019). Pharmacologic Inhibition of Epidermal Growth Factor Receptor Suppresses Nonalcoholic Fatty Liver Disease in a Murine Fast-Food Diet Model. Hepatology 70, 1546-1563. 10.1002/hep.30696.
26. Ohkubo, R., Mu, W. C., Wang, C. L., Song, Z., Barthez, M., Wang, Y., Mitchener, N., Abdullayev, R., Lee, Y. R., Ma, Y., et al. (2022). The hepatic integrated stress response suppresses the somatotroph axis to control liver damage in nonalcoholic fatty liver disease. Cell reports 41, 111803. 10.1016/j.celrep.2022.111803.
27. Kroy, D. C., Schumacher, F., Ramadori, P., Hatting, M., Bergheim, I., Gassler, N., Boekschoten, M. V., Muller, M., Streetz, K. L., and Trautwein, C. (2014). Hepatocyte specific deletion of c-Met leads to the development of severe non-alcoholic steatohepatitis in mice. Journal of hepatology 61, 883-890. 10.1016/j.jhep.2014.05.019.
28. Xiong, X., Kuang, H., Ansari, S., Liu, T., Gong, J., Wang, S., Zhao, X. Y., Ji, Y., Li, C., Guo, L., et al. (2019). Landscape of Intercellular Crosstalk in Healthy and NASH Liver Revealed by Single-Cell Secretome Gene Analysis. Molecular cell 75, 644-660 e645. 10.1016/j.molcel.2019.07.028.
29. Del Ben, M., Overi, D., Polimeni, L., Carpino, G., Labbadia, G., Baratta, F., Pastori, D., Noce, V., Gaudio, E., Angelico, F., and Mancone, C. (2018). Overexpression of the Vitronectin V10 Subunit in Patients with Nonalcoholic Steatohepatitis: Implications for Noninvasive Diagnosis of NASH. International journal of molecular sciences 19. 10.3390/ijms19020603.
30. Schwabe, R. F., Tabas, I., and Pajvani, U. B. (2020). Mechanisms of Fibrosis Development in Nonalcoholic Steatohepatitis. Gastroenterology 158, 1913-1928. 10.1053/j.gastro.2019.11.311.
31. Yang, L., Sun, Z., Li, J., Pan, X., Wen, J., Yang, J., Wang, Q., and Chen, P. (2022). Genetic Variants of Glycogen Metabolism Genes Were Associated With Liver PDFF Without Increasing NAFLD Risk. Frontiers in genetics 13, 830445. 10.3389/fgene.2022.830445.
32. Chella Krishnan, K., Floyd, R. R., Sabir, S., Jayasekera, D. W., Leon-Mimila, P. V., Jones, A. E., Cortez, A. A., Shravah, V., Peterfy, M., Stiles, L., et al. (2021). Liver Pyruvate Kinase Promotes NAFLD/NASH in Both Mice and Humans in a Sex-Specific Manner. Cell Mol Gastroenterol Hepatol 11, 389-406. 10.1016/j.jcmgh.2020.09.004.
33. Saed, C. T., Tabatabaei Dakhili, S. A., and Ussher, J. R. (2021). Pyruvate Dehydrogenase as a Therapeutic Target for Nonalcoholic Fatty Liver Disease. ACS Pharmacol Transl Sci 4, 582-588. 10.1021/acsptsci.0c00208.
34. Guilliams, M., Bonnardel, J., Haest, B., Vanderborght, B., Wagner, C., Remmerie, A., Bujko, A., Martens, L., Thone, T., Browaeys, R., et al. (2022). Spatial proteogenomics reveals distinct and evolutionarily conserved hepatic macrophage niches. Cell 185, 379-396 e338. 10.1016/j.cell.2021.12.018.
35. Faramia, J., Hao, Z., Mumphrey, M. B., Townsend, R. L., Miard, S., Carreau, A. M., Nadeau, M., Frisch, F., Baraboi, E. D., Grenier-Larouche, T., et al. (2021). IGFBP-2 partly mediates the early metabolic improvements caused by bariatric surgery. Cell Rep Med 2, 100248. 10.1016/j.xcrm.2021.100248.
36. Lin, Y. H., Wei, Y., Zeng, Q., Wang, Y., Pagani, C. A., Li, L., Zhu, M., Wang, Z., Hsieh, M. H., Corbitt, N., et al. (2023). IGFBP2 expressing midlobular hepatocytes preferentially contribute to liver homeostasis and regeneration. Cell Stem Cell 30, 665-676 e664. 10.1016/j.stem.2023.04.007.
37. Hedbacker, K., Birsoy, K., Wysocki, R. W., Asilmaz, E., Ahima, R. S., Farooqi, I. S., and Friedman, J. M. (2010). Antidiabetic effects of IGFBP2, a leptin-regulated gene. Cell metabolism 11, 11-22. 10.1016/j.cmet.2009.11.007.
38. Chen, X., Tang, Y., Chen, S., Ling, W., and Wang, Q. (2021). IGFBP-2 as a biomarker in NAFLD improves hepatic steatosis: an integrated bioinformatics and experimental study. Endocrine connections 10, 1315-1325. 10.1530/EC-21-0353.
39. Lu, D., Xia, Q., Yang, Z., Gao, S., Sun, S., Luo, X., Li, Z., Zhang, X., Han, S., Li, X., and Cao, M. (2021). ENO3 promoted the progression of NASH by negatively regulating ferroptosis via elevation of GPX4 expression and lipid accumulation. Annals of translational medicine 9, 661. 10.21037/atm-21-471.
40. Wang, J., Chen, L., Li, Y., and Guan, X. Y. (2011). Overexpression of cathepsin Z contributes to tumor metastasis by inducing epithelial-mesenchymal transition in hepatocellular carcinoma. PLoS One 6, e24967. 10.1371/journal.pone.0024967.
41. Zhang, J., Fan, N., and Peng, Y. (2018). Heat shock protein 70 promotes lipogenesis in HepG2 cells. Lipids in health and disease 17, 73. 10.1186/s12944-018-0722-8.
42. Chen, D., Dong, X., Chen, D., Lin, J., Lu, T., Shen, J., and Ye, H. (2023). Cdh1 plays a protective role in nonalcoholic fatty liver disease by regulating PPAR/PGC-1alpha signaling pathway. Biochemical and biophysical research communications 681, 13-19. 10.1016/j.bbrc.2023.09.038.
43. Henkel, A. S., Khan, S. S., Olivares, S., Miyata, T., and Vaughan, D. E. (2018). Inhibition of Plasminogen Activator Inhibitor 1 Attenuates Hepatic Steatosis but Does Not Prevent Progressive Nonalcoholic Steatohepatitis in Mice. Hepatol Commun 2, 1479-1492. 10.1002/hep4.1259.
44. Lee, S. M., Dorotea, D., Jung, I., Nakabayashi, T., Miyata, T., and Ha, H. (2017). TM5441, a plasminogen activator inhibitor-1 inhibitor, protects against high fat diet-induced non-alcoholic fatty liver disease. Oncotarget 8, 89746-89760. 10.18632/oncotarget.21120.
45. Day, K., Seale, L. A., Graham, R. M., and Cardoso, B. R. (2021). Selenotranscriptome Network in Non-alcoholic Fatty Liver Disease. Frontiers in nutrition 8, 744825. 10.3389/fnut.2021.744825.
46. Wang, L., Zhou, K., Wu, Q., Zhu, L., Hu, Y., Yang, X., and Li, D. (2023). Microanatomy of the metabolic associated fatty liver disease (MAFLD) by single-cell transcriptomics. J Drug Target 31, 421-432. 10.1080/1061186X.2023.2185626.
47. Sim, W. C., Lee, W., Sim, H., Lee, K. Y., Jung, S. H., Choi, Y. J., Kim, H. Y., Kang, K. W., Lee, J. Y., Choi, Y. J., et al. (2020). Downregulation of PHGDH expression and hepatic serine level contribute to the development of fatty liver disease. Metabolism 102, 154000. 10.1016/j.metabol.2019.154000.
48. Kuwashiro, S., Terai, S., Oishi, T., Fujisawa, K., Matsumoto, T., Nishina, H., and Sakaida, I. (2011). Telmisartan improves nonalcoholic steatohepatitis in medaka (Oryzias latipes) by reducing macrophage infiltration and fat accumulation. Cell and tissue research 344, 125-134. 10.1007/s00441-011-1132-7.
49. Moravcova, A., Cervinkova, Z., Kucera, O., Mezera, V., Rychtrmoc, D., and Lotkova, H. (2015). The effect of oleic and palmitic acid on induction of steatosis and cytotoxicity on rat hepatocytes in primary culture. Physiol Res 64, S627-636. 10.33549/physiolres.933224.
50. Enooku, K., Kondo, M., Fujiwara, N., Sasako, T., Shibahara, J., Kado, A., Okushin, K., Fujinaga, H., Tsutsumi, T., Nakagomi, R., et al. (2018). Hepatic IRS1 and ss-catenin expression is associated with histological progression and overt diabetes emergence in NAFLD patients. J Gastroenterol 53, 1261-1275. 10.1007/s00535-018-1472-0.
51. Honma, M., Sawada, S., Ueno, Y., Murakami, K., Yamada, T., Gao, J., Kodama, S., Izumi, T., Takahashi, K., Tsukita, S., et al. (2018). Selective insulin resistance with differential expressions of IRS-1 and IRS-2 in human NAFLD livers. Int J Obes (Lond) 42, 1544-1555. 10.1038/s41366-018-0062-9.
52. Liss, K. H., and Finck, B. N. (2017). PPARs and nonalcoholic fatty liver disease. Biochimie 136, 65-74. 10.1016/j.biochi.2016.11.009.
53. Montagner, A., Polizzi, A., Fouche, E., Ducheix, S., Lippi, Y., Lasserre, F., Barquissau, V., Regnier, M., Lukowicz, C., Benhamed, F., et al. (2016). Liver PPARalpha is crucial for whole-body fatty acid homeostasis and is protective against NAFLD. Gut 65, 1202-1214. 10.1136/gutjnl-2015-310798.
54. Ferre, P., and Foufelle, F. (2010). Hepatic steatosis: a role for de novo lipogenesis and the transcription factor SREBP-1c. Diabetes Obes Metab 12 Suppl 2, 83-92. 10.1111/j.1463-1326.2010.01275.x.
55. Pettinelli, P., and Videla, L. A. (2011). Up-regulation of PPAR-gamma mRNA expression in the liver of obese patients: an additional reinforcing lipogenic mechanism to SREBP-1c induction. J Clin Endocrinol Metab 96, 1424-1430. 10.1210/jc.2010-2129.
56. Moreno-Vedia, J., Girona, J., Ibarretxe, D., Masana, L., and Rodriguez-Calvo, R. (2022). Unveiling the Role of the Fatty Acid Binding Protein 4 in the Metabolic-Associated Fatty Liver Disease. Biomedicines 10. 10.3390/biomedicines10010197.
57. Oh, H. S., Rutledge, J., Nachun, D., Palovics, R., Abiose, O., Moran-Losada, P., Channappa, D., Urey, D. Y., Kim, K., Sung, Y. J., et al. (2023). Organ aging signatures in the plasma proteome track health and disease. Nature 624, 164-172. 10.1038/s41586-023-06802-1.
58. Govaere, O., Hasoon, M., Alexander, L., Cockell, S., Tiniakos, D., Ekstedt, M., Schattenberg, J. M., Boursier, J., Bugianesi, E., Ratziu, V., et al. (2023). A proteo-transcriptomic map of non-alcoholic fatty liver disease signatures. Nat Metab 5, 572-578. 10.1038/s42255-023-00775-1.
59. Perry, A. S., Amancherla, K., Huang, X., Lance, M. L., Farber-Eger, E., Gajjar, P., Amrute, J., Stolze, L., Zhao, S., Sheng, Q., et al. (2024). Clinical-transcriptional prioritization of the circulating proteome in human heart failure. Cell Rep Med, 101704. 10.1016/j.xcrm.2024.101704.
60. Niu, L., Geyer, P. E., Gupta, R., Santos, A., Meier, F., Doll, S., Wewer Albrechtsen, N. J., Klein, S., Ortiz, C., Uschner, F. E., et al. (2022). Dynamic human liver proteome atlas reveals functional insights into disease pathways. Molecular systems biology 18, e10947. 10.15252/msb.202210947.
61. Deczkowska, A., David, E., Ramadori, P., Pfister, D., Safran, M., Li, B., Giladi, A., Jaitin, D. A., Barboy, O., Cohen, M., et al. (2021). XCR1(+) type 1 conventional dendritic cells drive liver pathology in non-alcoholic steatohepatitis. Nat Med 27, 1043-1054. 10.1038/s41591-021-01344-3.
62. Rennert, K., Steinborn, S., Groger, M., Ungerbock, B., Jank, A. M., Ehgartner, J., Nietzsche, S., Dinger, J., Kiehntopf, M., Funke, H., et al. (2015). A microfluidically perfused three dimensional human liver model. Biomaterials 71, 119-131. 10.1016/j.biomaterials.2015.08.043.
63. Hassan, S., Sebastian, S., Maharjan, S., Lesha, A., Carpenter, A. M., Liu, X., Xie, X., Livermore, C., Zhang, Y. S., and Zarrinpar, A. (2020). Liver-on-a-Chip Models of Fatty Liver Disease. Hepatology 71, 733-740. 10.1002/hep.31106.
64. Du, K., Li, S., Li, C., Li, P., Miao, C., Luo, T., Qiu, B., and Ding, W. (2021). Modeling nonalcoholic fatty liver disease on a liver lobule chip with dual blood supply. Acta Biomater 134, 228-239. 10.1016/j.actbio.2021.07.013.
65. Levner, D., and Ewart, L. (2023). Integrating Liver-Chip data into pharmaceutical decision-making processes. Expert Opin Drug Discov 18, 1313-1320. 10.1080/17460441.2023.2255127.
66. Ewart, L., Apostolou, A., Briggs, S. A., Carman, C. V., Chaff, J. T., Heng, A. R., Jadalannagari, S., Janardhanan, J., Jang, K. J., Joshipura, S. R., et al. (2022). Performance assessment and economic analysis of a human Liver-Chip for predictive toxicology. Commun Med (Lond) 2, 154. 10.1038/s43856-022-00209-1.
67. Jang, K. J., Otieno, M. A., Ronxhi, J., Lim, H. K., Ewart, L., Kodella, K. R., Petropolis, D. B., Kulkarni, G., Rubins, J. E., Conegliano, D., et al. (2019). Reproducing human and cross-species drug toxicities using a Liver-Chip. Sci Transl Med 11. 10.1126/scitranslmed.aax5516.
68. Cong, Y., Han, X., Wang, Y., Chen, Z., Lu, Y., Liu, T., Wu, Z., Jin, Y., Luo, Y., and Zhang, X. (2020). Drug Toxicity Evaluation Based on Organ-on-a-chip Technology: A Review. Micromachines (Basel) 11. 10.3390/mi11040381.
69. Gori, M., Simonelli, M. C., Giannitelli, S. M., Businaro, L., Trombetta, M., and Rainer, A. (2016). Investigating Nonalcoholic Fatty Liver Disease in a Liver-on-a-Chip Microfluidic Device. PLoS One 11, e0159729. 10.1371/journal.pone.0159729.
70. European Association for the Study of the, L., European Association for the Study of, D., and European Association for the Study of, O. (2024). EASL-EASD-EASO Clinical Practice Guidelines on the management of metabolic dysfunction-associated steatotic liver disease (MASLD): Executive Summary. Diabetologia. 10.1007/s00125-024-06196-3.
71. Zhang, Y. N., Fowler, K. J., Hamilton, G., Cui, J. Y., Sy, E. Z., Balanay, M., Hooker, J. C., Szeverenyi, N., and Sirlin, C. B. (2018). Liver fat imaging—a clinical overview of ultrasound, CT, and MR imaging. The British journal of radiology 91, 20170959. 10.1259/bjr.20170959.
72. Sveinbjornsson, G., Ulfarsson, M. O., Thorolfsdottir, R. B., Jonsson, B. A., Einarsson, E., Gunnlaugsson, G., Rognvaldsson, S., Arnar, D. O., Baldvinsson, M., Bjarnason, R. G., et al. (2022). Multiomics study of nonalcoholic fatty liver disease. Nature genetics 54, 1652-1663. 10.1038/s41588-022-01199-5.
73. Yoo, D., Divard, G., Raynaud, M., Cohen, A., Mone, T. D., Rosenthal, J. T., Bentall, A. J., Stegall, M. D., Naesens, M., Zhang, H., et al. (2024). A Machine Learning-Driven Virtual Biopsy System For Kidney Transplant Patients. Nature communications 15, 554. 10.1038/s41467-023-44595-z.
74. Ng, C. H., Lim, W. H., Hui Lim, G. E., Hao Tan, D. J., Syn, N., Muthiah, M. D., Huang, D. Q., and Loomba, R. (2023). Mortality Outcomes by Fibrosis Stage in Nonalcoholic Fatty Liver Disease: A Systematic Review and Meta-analysis. Clin Gastroenterol Hepatol 21, 931-939 e935. 10.1016/j.cgh.2022.04.014.
75. Simon, T. G., Roelstraete, B., Khalili, H., Hagstrom, H., and Ludvigsson, J. F. (2021). Mortality in biopsy-confirmed nonalcoholic fatty liver disease: results from a nationwide cohort. Gut 70, 1375-1382. 10.1136/gutjnl-2020-322786.
76. Targher, G., and Byrne, C. D. (2013). Clinical Review: Nonalcoholic fatty liver disease: a novel cardiometabolic risk factor for type 2 diabetes and its complications. J Clin Endocrinol Metab 98, 483-495. 10.1210/jc.2012-3093.
77. Liu, C., Liu, T., Zhang, Q., Jia, P., Song, M., Zhang, Q., Ruan, G., Ge, Y., Lin, S., Wang, Z., et al. (2023). New-Onset Age of Nonalcoholic Fatty Liver Disease and Cancer Risk. JAMA Netw Open 6, e2335511. 10.1001/jamanetworkopen.2023.35511.
78. Wagenknecht, L. E., Perkins, L. L., Cutter, G. R., Sidney, S., Burke, G. L., Manolio, T. A., Jacobs, D. R., Jr., Liu, K. A., Friedman, G. D., Hughes, G. H., and et al. (1990). Cigarette smoking behavior is strongly related to educational status: the CARDIA study. Preventive medicine 19, 158-169.
79. Dyer, A. R., Cutter, G. R., Liu, K. Q., Armstrong, M. A., Friedman, G. D., Hughes, G. H., Dolce, J. J., Raczynski, J., Burke, G., and Manolio, T. (1990). Alcohol intake and blood pressure in young adults: the CARDIA Study. Journal of clinical epidemiology 43, 1-13.
80. Bild, D. E., Jacobs, D. R., Jr., Sidney, S., Haskell, W. L., Anderssen, N., and Oberman, A. (1993). Physical activity in young black and white women. The CARDIA Study. Ann Epidemiol 3, 636-644.
81. Sidney, S., Jacobs, D. R., Jr., Haskell, W. L., Armstrong, M. A., Dimicco, A., Oberman, A., Savage, P. J., Slattery, M. L., Sternfeld, B., and Van Horn, L. (1991). Comparison of two methods of assessing physical activity in the Coronary Artery Risk Development in Young Adults (CARDIA) Study. Am J Epidemiol 133, 1231-1245.
82. Sun, B. B., Chiou, J., Traylor, M., Benner, C., Hsu, Y. H., Richardson, T. G., Surendran, P., Mahajan, A., Robins, C., Vasquez-Grinnell, S. G., et al. (2023). Plasma proteomic associations with genetics and health in the UK Biobank. Nature 622, 329-338. 10.1038/s41586-023-06592-6.
83. Jiao, J., Watt, G. P., Lee, M., Rahbar, M. H., Vatcheva, K. P., Pan, J. J., McCormick, J. B., Fisher-Hoch, S. P., Fallon, M. B., and Beretta, L. (2016). Cirrhosis and Advanced Fibrosis in Hispanics in Texas: The Dominant Contribution of Central Obesity. PLoS One 11, e0150978. 10.1371/journal.pone.0150978.
84. de Ledinghen, V., Vergniol, J., Capdepont, M., Chermak, F., Hiriart, J. B., Cassinotto, C., Merrouche, W., Foucher, J., and Brigitte le, B. (2014). Controlled attenuation parameter (CAP) for the diagnosis of steatosis: a prospective study of 5323 examinations. J Hepatol 60, 1026-1031. 10.1016/j.jhep.2013.12.018.
85. Wilman, H. R., Kelly, M., Garratt, S., Matthews, P. M., Milanesi, M., Herlihy, A., Gyngell, M., Neubauer, S., Bell, J. D., Banerjee, R., and Thomas, E. L. (2017). Characterisation of liver fat in the UK Biobank cohort. PLoS One 12, e0172921. 10.1371/journal.pone.0172921.
86. Browning, J. D., Szczepaniak, L. S., Dobbins, R., Nuremberg, P., Horton, J. D., Cohen, J. C., Grundy, S. M., and Hobbs, H. H. (2004). Prevalence of hepatic steatosis in an urban population in the United States: impact of ethnicity. Hepatology 40, 1387-1395. 10.1002/hep.20466.
87. Watt, G. P., De La Cerda, I., Pan, J. J., Fallon, M. B., Beretta, L., Loomba, R., Lee, M., McCormick, J. B., and Fisher-Hoch, S. P. (2020). Elevated Glycated Hemoglobin Is Associated With Liver Fibrosis, as Assessed by Elastography, in a Population-Based Study of Mexican Americans. Hepatol Commun 4, 1793-1801. 10.1002/hep4.1603.
88. Han, Z., Xiao, Z., Kalantar-Zadeh, K., Moradi, H., Shafi, T., Waikar, S. S., Quarles, L. D., Yu, Z., Tin, A., Coresh, J., and Kovesdy, C. P. (2018). Validation of a Novel Modified Aptamer-Based Array Proteomic Platform in Patients with End-Stage Renal Disease. Diagnostics (Basel) 8. 10.3390/diagnostics8040071.
89. Ren, Y., Ruan, P., Segal, M., Dobre, M., Schelling, J. R., Banerjee, U., Shafi, T., Ganz, P., Dubin, R. F., and Investigators, C. S. (2023). Evaluation of a large-scale aptamer proteomics platform among patients with kidney failure on dialysis. PLoS One 18, e0293945. 10.1371/journal.pone.0293945.
90. Candia, J., Daya, G. N., Tanaka, T., Ferrucci, L., and Walker, K. A. (2022). Assessment of variability in the plasma 7k SomaScan proteomics assay. Sci Rep 12, 17147. 10.1038/s41598-022-22116-0.
91. Lollo, B., Steele, F., and Gold, L. (2014). Beyond antibodies: new affinity reagents to unlock the proteome. Proteomics 14, 638-644. 10.1002/pmic.201300187.
92. Rohloff, J. C., Gelinas, A. D., Jarvis, T. C., Ochsner, U. A., Schneider, D. J., Gold, L., and Janjic, N. (2014). Nucleic Acid Ligands With Protein-like Side Chains: Modified Aptamers and Their Use as Diagnostic and Therapeutic Agents. Mol Ther Nucleic Acids 3, e201. 10.1038/mtna.2014.49.
93. Fredriksson, S., Dixon, W., Ji, H., Koong, A. C., Mindrinos, M., and Davis, R. W. (2007). Multiplexed protein detection by proximity ligation for cancer biomarker validation. Nat Methods 4, 327-329. 10.1038/nmeth1020.
94. van der Poorten, D., Samer, C. F., Ramezani-Moghadam, M., Coulter, S., Kacevska, M., Schrijnders, D., Wu, L. E., McLeod, D., Bugianesi, E., Komuta, M., et al. (2013). Hepatic fat loss in advanced nonalcoholic steatohepatitis: are alterations in serum adiponectin the cause? Hepatology 57, 2180-2188. 10.1002/hep.26072.
95. Storey, J. D., and Tibshirani, R. (2003). Statistical significance for genomewide studies. Proc Natl Acad Sci USA 100, 9440-9445. 10.1073/pnas.1530509100.
96. Robinson, M. D., and Oshlack, A. (2010). A scaling normalization method for differential expression analysis of RNA-seq data. Genome Biol 11, R25. 10.1186/gb-2010-11-3-r25.
97. Chatterjee, E., Rodosthenous, R. S., Kujala, V., Gokulnath, P., Spanos, M., Lehmann, H. I., de Oliveira, G. P., Shi, M., Miller-Fleming, T. W., Li, G., et al. (2023). Circulating extracellular vesicles in human cardiorenal syndrome promote renal injury in a kidney-on-chip system. JCI Insight 8. 10.1172/jci.insight.165172.
98. Yu, G., Wang, L. G., Han, Y., and He, Q. Y. (2012). clusterProfiler: an R package for comparing biological themes among gene clusters. OMICS 16, 284-287. 10.1089/omi.2011.0118.
99. Shannon, P., Markiel, A., Ozier, O., Baliga, N. S., Wang, J. T., Ramage, D., Amin, N., Schwikowski, B., and Ideker, T. (2003). Cytoscape: a software environment for integrated models of biomolecular interaction networks. Genome Res 13, 2498-2504. 10.1101/gr.1239303.
100. Jain, A., and Tuteja, G. (2019). TissueEnrich: Tissue-specific gene enrichment analysis. Bioinformatics 35, 1966-1967. 10.1093/bioinformatics/bty890.
101. Uhlen, M., Fagerberg, L., Hallstrom, B. M., Lindskog, C., Oksvold, P., Mardinoglu, A., Sivertsson, A., Kampf, C., Sjostedt, E., Asplund, A., et al. (2015). Proteomics. Tissue-based map of the human proteome. Science 347, 1260419. 10.1126/science.1260419.
102. Jiang, L., Wang, M., Lin, S., Jian, R., Li, X., Chan, J., Dong, G., Fang, H., Robinson, A. E., Consortium, G. T., and Snyder, M. P. (2020). A Quantitative Proteome Map of the Human Body. Cell 183, 269-283 e219. 10.1016/j.cell.2020.08.036.
103. Gonzales, T. I., Westgate, K., Strain, T., Hollidge, S., Jeon, J., Christensen, D. L., Jensen, J., Wareham, N. J., and Brage, S. (2021). Cardiorespiratory fitness assessment using risk-stratified exercise testing and dose-response relationships with disease outcomes. Sci Rep 11, 15315. 10.1038/s41598-021-94768-3.
104. Wu, P., Gifford, A., Meng, X., Li, X., Campbell, H., Varley, T., Zhao, J., Carroll, R., Bastarache, L., Denny, J. C., et al. (2019). Mapping ICD-10 and ICD-10-CM Codes to Phecodes: Workflow Development and Initial Evaluation. JMIR Med Inform 7, e14325. 10.2196/14325.

It will be understood that various details of the presently disclosed subject matter can be changed without departing from the scope of the subject matter disclosed herein. Furthermore, the foregoing description is for the purpose of illustration only, and not for the purpose of limitation.

What is claimed is:

1. A method of diagnosing or predicting metabolic dysfunction-associated steatotic liver disease (MASLD) or progression thereof in a subject, comprising:
(a) obtaining a plasma or serum sample from the subject;
(b) quantifying concentrations of a plurality of biomarkers selected from the group consisting of:
(i) DLD, CFD, SMPDL3A, APOA5, HEXB, GUSB, ADH1A, UGDH, CHMP1A, AKR1C4, RBP5, IHH, STARD10, GLUD1, NRP1, IL1RN, MLN, PTPRS, SHBG, PCSK9, and HGD; or
(ii) ANGPT2, ANGPTL3, CES1, CHMP1A, CSTZ, DNAJB1, FCGR3B, GLO1, GSTA1, GUSB, IGFBP3, IL1RN, MLN, NRP1, PCSK9, PTPRS, RBP5, RELT, SEMA4D, SMPD1, and SORD;
(c) generating a MASLD risk score based on the quantified concentrations; and
(d) identifying the subject as having MASLD or an increased risk thereof when the MASLD risk score exceeds a threshold value.

2. The method of claim 1, wherein obtaining the plasma or serum sample comprises processing whole blood to isolate plasma or serum and performing protein denaturation and/or enzymatic digestion prior to biomarker quantification.

3. The method of claim 1, wherein quantifying concentrations of the plurality of biomarkers comprises performing a multiplexed mass spectrometry assay configured to detect multiple biomarkers in a single analytical run, optionally using isobaric labeling reagents such as tandem mass tags (TMT) or equivalent multiplexing strategies.

4. The method of claim 1, wherein quantifying concentrations of the plurality of biomarkers is performed using an automated analytical device configured with a calibration curve tailored for MASLD biomarker ranges.

5. The method of claim 1, further comprising validating the MASLD risk score using a liver-on-a-chip system configured to model hepatic steatosis under controlled conditions.

6. The method of claim 1, wherein generating the MASLD risk score comprises applying a multivariable model that assigns biomarker-specific coefficients derived from a regression analysis or machine learning algorithm trained on MASLD phenotypes and validated using receiver operating characteristic analysis.

7. The method of claim 1, wherein generating the MASLD risk score further comprises integrating clinical covariates selected from age, body mass index, and fasting glucose level into the multivariable model.

8. The method of claim 1, wherein generating the MASLD risk score comprises applying a multivariable model comprising biomarker-specific coefficients derived from a regression analysis or machine learning algorithm trained on MASLD phenotypes.

9. The method of claim 1, wherein generating the MASLD risk score is performed by a computing device executing instructions stored on a non-transitory computer-readable medium.

10. The method of claim 1, wherein the threshold value is determined using receiver operating characteristic (ROC) analysis for MASLD classification.

11. The method of claim 1, further comprising administering to the subject a therapeutic agent or intervention for treating MASLD when the MASLD risk score exceeds the threshold value.

12. The method of claim 11, wherein the therapeutic agent is selected grom the group consisting of sodium-glucose cotransporter 2 (SGLT2) inhibitors, glucagon-like peptide-1 (GLP-1) receptor agonists, dual glucose-dependent insulinotropic polypeptide and glucagon-like peptide-1 (GIP/GLP-1) receptor agonists, thiazolidinediones (TZDs), biguanides, thyroid hormone receptor beta (THR-β) agonists, fibroblast growth factor (FGF) analogs, farnesoid X receptor (FXR) agonists, peroxisome proliferator-activated receptor (PPAR) agonists, acetyl-coenzyme A carboxylase (ACC) inhibitors, diacylglycerol O-acyltransferase 2 (DGAT2) inhibitors, C—C chemokine receptor type 2 and type 5 (CCR2/CCR5) antagonists, antioxidants, and statins.

13. A kit for diagnosing or predicting metabolic dysfunction-associated steatotic liver disease (MASLD) in a subject, comprising:
(a) a plurality of reagents configured for detection of a plurality of biomarkers selected from the group consisting of
(i) DLD, CFD, SMPDL3A, APOA5, HEXB, GUSB, ADH1A, UGDH, CHMP1A, AKR1C4, RBP5, IHH, STARD10, GLUD1, NRP1, IL1RN, MLN, PTPRS, SHBG, PCSK9, and HGD; or
(ii) ANGPT2, ANGPTL3, CES1, CHMP1A, CSTZ, DNAJB1, FCGR3B, GLO1, GSTA1, GUSB, IGFBP3, IL1RN, MLN, NRP1, PCSK9, PTPRS, RBP5, RELT, SEMA4D, SMPD1, and SORD; and
(b) instructions, stored on a non-transitory computer-readable medium or provided in printed form, for calculating a MASLD risk score as a combination of said concentrations and predetermined coefficients derived from a regression analysis or machine learning algorithm trained on MASLD phenotypes.

14. The kit of claim 13, wherein the plurality of reagents is provided in a multiplexed assay cartridge configured for simultaneous detection of at least two biomarkers in a single analytical run.

15. The kit of claim 13, wherein the multiplexed assay cartridge comprises a non-standard calibration curve optimized for MASLD biomarker ranges.

16. The kit of claim 13, wherein the instructions stored on the non-transitory computer-readable medium comprise executable code for applying a multivariable model comprising biomarker-specific coefficients derived from a regression analysis trained on MASLD phenotypes.

17. The kit of claim 13, wherein the instructions further comprise code for comparing the MASLD risk score to a threshold value determined from a receiver operating characteristic curve for MASLD classification.

18. The kit of claim 13, wherein the instructions further comprise code for integrating clinical covariates selected from age, body mass index, and fasting glucose level into the MASLD risk score calculation.

19. A computer-implemented method of diagnosing or predicting risk of MASLD in a subject, comprising:
(a) receiving as input quantified concentrations of a plurality of biomarkers selected from
(i) DLD, CFD, SMPDL3A, APOA5, HEXB, GUSB, ADH1A, UGDH, CHMP1A, AKR1C4, RBP5, IHH, STARD10, GLUD1, NRP1, IL1RN, MLN, PTPRS, SHBG, PCSK9, and HGD; or
(ii) ANGPT2, ANGPTL3, CES1, CHMP1A, CSTZ, DNAJB1, FCGR3B, GLO1, GSTA1, GUSB, IGFBP3, IL1RN, MLN, NRP1, PCSK9, PTPRS, RBP5, RELT, SEMA4D, SMPD1, and SORD;
(b) applying a multivariable model trained on MASLD phenotypes to said concentrations, wherein the model comprises biomarker-specific coefficients derived from regression analysis or machine learning; and
(c) generating and outputting a MASLD risk score indicative of the subject's MASLD status.

20. The method of claim 19, wherein applying the multivariable model comprises executing a trained machine learning algorithm selected from gradient boosting regression, random forest regression, or neural network regression.
